(19) United States
(12) Patent Application Publication
(10) Pub. No.: US 2025/0032493 A1
(43) Pub. Date: Jan. 30, 2025

(54) Methods for Treating Diseases Using MALT1 Inhibitors

(71) Applicant: RARIFIED BIOSCIENCES, INC., Cambridge, MA (US)

(72) Inventors: Subhabrata Biswas, Cambridge, MA (US); Patricia Elaine Rao, Cambridge, MA (US); Barbara Saxton Fox, Cambridge, MA (US); Dania Mounir Rabah, Cambridge, MA (US); Andrew John Long, Cambridge, MA (US)

(73) Assignee: RARIFIED BIOSCIENCES, INC., Cambridge, MA (US)

(21) Appl. No.: 18/715,889

(22) PCT Filed: Dec. 9, 2022
(86) PCT No.: PCT/US2022/052450

§ 371 Date: Jun. 3, 2024

Related U.S. Application Data
(60) Provisional application No. 63/288,081, filed on Dec. 10, 2021.
(60) Provisional application No. 63/288,083, filed on Dec. 10, 2021.
(60) Provisional application No. 63/288,085, filed on Dec. 10, 2021.
(60) Provisional application No. 63/306,655, filed on Feb. 4, 2022.
(60) Provisional application No. 63/306,657, filed on Feb. 4, 2022.
(60) Provisional application No. 63/306,660, filed on Feb. 4, 2022.

(51) Int. Cl. A61K 31/519; A61K 31/4375; A61K 31/4709; A61K 31/5025; A61P 19/02; A61P 37/06

(52) U.S. Cl. CPC A61K 31/519; A61K 31/4375; A61K 31/4709; A61K 31/5025; A61P 19/02; A61P 37/06

(57) ABSTRACT

Provided herein are MALT1 inhibitors and the administration of MALT1 inhibitors in subjects such that the efficacy of MALT1 inhibitors is decoupled from the reduction of regulatory T cells. Therefore, the MALT1 inhibitors and methods for administering the MALT1 inhibitors enable the treatment of diseases (e.g., autoimmune diseases) while avoiding reduction of Tregs.

### CROSS-REFERENCE TO RELATED APPLICATIONS

This application claims the benefit of and priority to U.S. Provisional Patent Application No. 63/288,081 filed Dec. 10, 2021, U.S. Provisional Patent Application No. 63/288,083 filed Dec. 10, 2021, U.S. Provisional Patent Application No. 63/288,085 filed Dec. 10, 2021, U.S. Provisional Patent Application No. 63/306,655 filed Feb. 4, 2022, U.S. Provisional Patent Application No. 63/306,657 filed Feb. 4, 2022, U.S. Provisional Patent Application No. 63/306,660 filed Feb. 4, 2022, the entire disclosure of each of which is hereby incorporated by reference in its entirety for all purposes.

### BACKGROUND OF THE INVENTION

Mucosa associated lymphoid tissue lymphoma translocation protein 1 (MALT1) is an intracellular signaling protein, known from innate (e.g., natural killer cells NK, dendritic cells DC, and mast cells) and adaptive immune cells (e.g., T cells and B cells). MALT1 plays an essential role in influencing immune responses. For example, in T cell receptor signaling, MALT1 mediates nuclear factor κB (NFKB) signaling, leading to T cell activation and proliferation. Accordingly, MALT1 is of interest in the mechanism of autoimmune and inflammatory pathologies. In addition, constitutive (dysregulated) MALT1 activity is associated with cancers such as MALT lymphoma and activated B cell-like diffuse large B Cell lymphoma (ABC-DLBCL). Modulators of MALT1 activity may be useful as potential therapeutics.

### SUMMARY OF THE INVENTION

Provided herein are compounds designed to act as MALT1 inhibitors. Further disclosed herein are the administration of MALT1 inhibitors in subjects such that the efficacy of MALT1 inhibitors is decoupled from the reduction of regulatory T cells (Tregs). Generally, Tregs control the immune response to self and foreign antigens and assist in preventing autoimmune disease. Therefore, the MALT1 inhibitors and methods for administering the MALT1 inhibitors enable the treatment of diseases (e.g., autoimmune diseases) while avoiding reduction of Tregs. In particular embodiments, MALT1 inhibitors are effective for treating chronic diseases or chronic disorders, including any of chronic graft-versus-host disease (cGHVD), delayed-type hypersensitivity, psoriatic arthritis, primary sclerosing cholangitis, multiple sclerosis, inflammatory bowel disease, Crohn's Disease, ulcerative colitis, psoriasis, Lupus, Sjögren's syndrome, scleritis, or rheumatoid arthritis

Disclosed herein is a method for treating a chronic disorder, the method comprising administering to the subject a MALT1 inhibitor, wherein following administration to the subject, the MALT1 inhibitor achieves a time over an IC50 blood concentration target from about 4 hours to about 20 hours per 24 hours. In various embodiments, following administration to the subject, the MALT1 inhibitor achieves a time over the blood concentration target from about 6 hours to about 18 hours per 24 hours. In various embodiments, following administration to the subject, the MALT1 inhibitor achieves a time over the blood concentration target from about 8 hours to about 16 hours per 24 hours. In various embodiments, following administration to the subject, the MALT1 inhibitor achieves a time over the blood concentration target from about 10 hours to about 14 hours per 24 hours.

In various embodiments, the MALT1 inhibitor is administered at a dose from about 1 mg/kg to about 6 mg/kg. In various embodiments, the MALT1 inhibitor is administered at a dose from about 2 mg/kg to about 5 mg/kg. In various embodiments, the MALT1 inhibitor is administered at a dose of about 3 mg/kg. In various embodiments, following administration to the subject, the MALT1 inhibitor achieves a time over a IC50 blood concentration target from about 12 hours to about 24 hours per 24 hours. In various embodiments, following the administration to the subject, the MALT1 inhibitor achieves a time over the IC50 blood concentration target from about 16 hours to about 24 hours per 24 hours. In various embodiments, following the administration to the subject, the MALT1 inhibitor achieves a time over the IC50 blood concentration target from about 22 hours to about 24 hours per 24 hours. In various embodiments, following the administration to the subject, the MALT1 inhibitor achieves a time over a IC90 blood concentration target from about 1 hour to about 15 hours per 24 hours. In various embodiments, following the administration to the subject, the MALT1 inhibitor achieves a time over a IC90 blood concentration target from about 3 hours to about 12 hours per 24 hours. In various embodiments, following the administration to the subject, the MALT1 inhibitor achieves a time over a IC90 blood concentration target from about 6 hours to about 10 hours per 24 hours.

In various embodiments, the MALT1 inhibitor is administered at a dose from about 8 mg/kg to about 20 mg/kg. In various embodiments, the MALT1 inhibitor is administered at a dose from about 9 mg/kg to about 15 mg/kg. In various embodiments, the MALT1 inhibitor is administered at a dose of about 10 mg/kg. In various embodiments, following administration to the subject, the MALT1 inhibitor achieves a time over a IC90 blood concentration target from about 6 hours to about 24 hours per 24 hours. In various embodiments, the MALT1 inhibitor achieves a time over a IC90 blood concentration target from about 12 hours to about 24 hours per 24 hours. In various embodiments, the MALT1 inhibitor achieves a time over a IC90 blood concentration target from about 18 hours to about 24 hours per 24 hours. In various embodiments, the MALT1 inhibitor achieves a time over a IC90 blood concentration target from about 21 hours to about 24 hours per 24 hours.

In various embodiments, the MALT1 inhibitor is administered at a dose from about 8 mg/kg to about 20 mg/kg. In various embodiments, the MALT1 inhibitor is administered at a dose from about 9 mg/kg to about 15 mg/kg. In various embodiments, the MALT1 inhibitor is administered at a dose of about 10 mg/kg.

Additionally disclosed herein is a method for treating a chronic disorder, the method comprising administering to the subject a MALT1 inhibitor, wherein following administration to the subject, the MALT1 inhibitor achieves a log₁₀(AUC) from about 0.5 μg*hr/mL to about 2.0 μg*hr/mL. In various embodiments, following administration to the subject, the MALT1 inhibitor achieves a log₁₀(AUC) from about 1.0 μg*hr/mL to about 1.75 μg*hr/mL. In various embodiments, following administration to the subject, the MALT1 inhibitor achieves a log₁₀(AUC) from about 1.25 μg*hr/mL to about 1.50 μg*hr/mL. In various embodiments, following administration of the MALT1 inhibitor to the subject, a level of Tregs of the subject remains at least 60% of a level prior to administration. In various embodiments, following administration of the MALT1 inhibitor to the subject, a level of Tregs of the subject remains at least 70% of a level prior to administration. In various embodiments, following administration of the MALT1 inhibitor to the subject, a level of Tregs of the subject remains at least 80% of a level prior to administration. In various embodiments, following administration of the MALT1 inhibitor to the subject, a level of Tregs of the subject remains at least 90% of a level prior to administration. In various embodiments, following administration of the MALT1 inhibitor to the subject, a level of Tregs of the subject remains at least 95% of a level prior to administration.

Additionally disclosed herein is a method for treating a chronic disorder, the method comprising administering to the subject a MALT1 inhibitor, wherein the MALT1 inhibitor is administered at a dose between from about 1 mg/kg to about 100 mg/kg. In various embodiments, the MALT1 inhibitor is administered at a dose between from about 20 mg/kg to about 40 mg/kg. In various embodiments, the MALT1 inhibitor is administered at a dose between from about 25 mg/kg to about 35 mg/kg. In various embodiments, the MALT1 inhibitor is administered at a dose of about 30 mg/kg. In various embodiments, the MALT1 inhibitor is administered at a dose between from about 5 mg/kg to about 15 mg/kg. In various embodiments, the MALT1 inhibitor is administered at a dose between from about 8 mg/kg to about 12 mg/kg. In various embodiments, the MALT1 inhibitor is administered at a dose of about 10 mg/kg. In various embodiments, the MALT1 inhibitor is administered at a dose between from about 2 mg/kg to about 10 mg/kg In various embodiments, the MALT1 inhibitor is administered at a dose from about 2 mg/kg to about 5 mg/kg. In various embodiments, the MALT1 inhibitor is administered at a dose of about 3 mg/kg. In various embodiments, the MALT1 inhibitor is administered at a dose between from about 1 mg/kg to about 5 mg/kg. In various embodiments, the MALT1 inhibitor is administered at a dose from about 1 mg/kg to about 3 mg/kg. In various embodiments, the MALT1 inhibitor is administered at a dose of about 1 mg/kg. In various embodiments, the MALT1 inhibitor is administered intravenously. In various embodiments, the MALT1 inhibitor is locally administered.

In various embodiments, the MALT1 inhibitor is administered daily for between 5 to 20 days. In various embodiments, the MALT1 inhibitor is administered daily for between 5 to 8 days. In various embodiments, the MALT1 inhibitor is administered daily for 7 days. In various embodiments, the MALT1 inhibitor is administered daily for between 10 to 15 days. In various embodiments, the MALT1 inhibitor is administered daily for 14 days. In various embodiments, the MALT1 inhibitor is administered for one or more cycles, wherein a cycle comprises: administering the MALT1 inhibitor once per day for between 1-2 weeks followed by no treatment for 1-2 weeks. In various embodiments, a cycle comprises: administering the MALT1 inhibitor once per day for 2 weeks followed by no treatment for 1 week. In various embodiments, reduction of a level of Tregs of the subject with the chronic disorder following administration of the MALT1 inhibitor is lower in comparison to reduction of a level of Tregs of a healthy subject who receives the MALT1 inhibitor.

Additionally disclosed herein is a method for treating a chronic disorder, the method comprising administering to the subject a MALT1 inhibitor, wherein the subject has been previously identified as having elevated IL-2 relative to a reference. Additionally disclosed herein is a method for treating a chronic disorder, the method comprising administering to the subject a MALT1 inhibitor, wherein the subject has been previously identified as having IL-15 relative to a reference. Additionally disclosed herein is a method for treating a chronic disorder, the method comprising administering to the subject a MALT1 inhibitor, wherein the subject has been previously identified as having elevated IL-7 relative to a reference.

Additionally disclosed herein is a method for treating a chronic disorder, the method comprising administering to the subject a MALT1 inhibitor in combination with a second agent comprising any one of IL-2, IL-15, and IL-7. In various embodiments, the second agent comprises IL-2, and wherein the IL-2 is administered at a dose from about 10,000 International Units (IUs) to about 50,000 International Units (IUs). In various embodiments, the second agent comprises IL-2, and wherein the second agent is administered at a dose from about 20,000 International Units (IUs) to about 40,000 International Units (IUs). In various embodiments, the second agent comprises IL-2, and wherein the second agent is administered at a dose of about 30,000 International Units (IUs). In various embodiments, the second agent comprises IL-2, and wherein the IL-2 is administered at a dose from about 100,000 International Units (IUs) to about 5 million International Units (IUs). In various embodiments, the second agent comprises IL-2, and wherein the IL-2 is administered at a dose from about 500,000 International Units (IUs) to about 4.5 million International Units (IUs). In various embodiments, the second agent comprises IL-2, and wherein the IL-2 is administered at a dose from about 1 million International Units (IUs) to about 4 million International Units (IUs). In various embodiments, the second agent comprises IL-2, and wherein the IL-2 is administered at a dose from about 2 million International Units (IUs) to about 3 million International Units (IUs). In various embodiments, the second agent comprises IL-2, and wherein the IL-2 is administered at a dose of about 3 million International Units (IUs).

Additionally disclosed herein is a method for treating a chronic disorder, the method comprising administering to the subject a MALT1 inhibitor, wherein efficacy of the MALT1 inhibitor is decoupled from its depletive effect on Tregs. In various embodiments, efficacy of the MALT1 inhibitor is represented by a reduction of at least about a 50% in a clinical score, and wherein the depletive effect of the MALT1 inhibitor on Tregs is represented by at least about a 40% reduction in Tregs.

Additionally disclosed herein is a method for treating a chronic disorder, the method comprising administering to the subject a MALT1 inhibitor, wherein following administration of the MALT1 inhibitor to the subject, a level of Tregs of the subject remains at least 60% of a level prior to administration. In various embodiments, following administration of the MALT1 inhibitor to the subject, a level of Tregs of the subject remains at least 60%, at least 70%, at least 80%, at least 90%, or at least 95% of a level prior to administration. In various embodiments, the chronic disorder is graft-versus-host disease (GHVD). In various embodiments, the graft-versus-host disease (GHVD) is sclerodermatous GVHD (scGVHD). In various embodiments, the chronic disorder is psoriatic arthritis. In various embodiments, the chronic disorder is primary sclerosing cholangitis. In various embodiments, the chronic disorder is multiple sclerosis. In various embodiments, the chronic disorder is inflammatory bowel disease. In various embodiments, the inflammatory bowel disease is Crohn's Disease. In various embodiments, the inflammatory bowel disease is ulcerative colitis. In various embodiments, the chronic disorder is psoriasis. In various embodiments, the chronic disorder is Lupus. In various embodiments, the chronic disorder is Sjögren's syndrome. In various embodiments, the chronic disorder is scleritis. In various embodiments, the chronic disorder is rheumatoid arthritis. In various embodiments, the chronic disorder is delayed-type hypersensitivity.

### BRIEF DESCRIPTION OF THE FIGURES

These and other features, aspects, and advantages of the present invention will become better understood with regard to the following description and accompanying drawings.

FIG. 1 depicts an example method for identifying candidate subjects for receiving a MALT1 inhibitor, in accordance with an embodiment.

FIG. 2 depicts restoration of IL-2 signaling (pStat5) in MALT1 conditional knockout mice with the addition of exogenous IL-2.

FIG. 3A depicts example MALT1 inhibitor structures.

FIG. 3B shows suppression of proinflammatory cytokines-IFNγ, IL-2 and TNFα from CD45RO+ memory T cells activated via cross-linking of the T-cell receptor (α-CD3/-CD28/-CD2) for 24 h in the presence of increasing concentrations of MALT1 inhibitor.

FIG. 3C shows suppression of IL-17a from CD45RO memory T-cells activated via cross-linking of the T-cell receptor+co-stimulation (α-CD3/-CD28/-CD2) for 48 h in the presence increasing concentrations of MALT1 inhibitor.

FIG. 3D shows human B-cell proliferation induced via IgM/CD40L stimulation was attenuated by inhibition of MALT1.

FIG. 3E shows production of IL-6 and TNFα from immune-complex stimulated human monocyte derived macrophages was inhibited with MALT1 inhibition.

FIG. 3F shows dose-dependent impact on protease activity by MALT1i-dependent cleavage of HOIL-1 substrate as determined by immunoblotting is shown. Shaded area corresponds to concentration of MALT1i necessary to achieve 50-90% target coverage as determined from the human whole blood assay.

FIG. 4A shows that clinical scores were reduced in rats (n=8/group) treated with MALT1i prior to immunization with collagen (Day 0, 7) with indicated doses once daily for four weeks (prophylactic, left). Clinical scores measured three times per week starting from Day 14 are plotted (mean±S.E.M) for each group. Total disease burden over time is represented by plotting the calculated area under curve (AUC) across the treatment groups (right).

FIG. 4B shows clinical scores of animals immunized with collagen, randomized on day 12 and treated with MALT1i for two weeks starting from Day 14 (therapeutic regimen). Clinical scores measured three times per week are plotted (mean±S.E.M) for each group (left). Total disease burden over time is represented by plotting the calculated area under curve (AUC) across the treatment groups (right). Significant difference from the vehicle treated group was calculated via One-way ANOVA using Graphpad Prism, *p<0.05, **p<0.01, ***p<0.001, ****p<0.0001,

FIG. 4C shows plasma concentration of MALT1 inhibitor measured between 0-24 h following last dose. Rat whole blood potency (IC₅₀ and IC₉₀, see Table 2) values indicated as dotted lines.

FIG. 4D shows that therapeutic treatment with MALT1 inhibitor suppresses proinflammatory cytokine and auto-antibody production in rat model of collagen induced arthritis (CIA). Proinflammatory cytokines in the plasma (top row) and in synovium (bottom row) harvested from whole blood and knee joints, respectively, were measured at study termination. Cytokines that showed significant change in MALT1i treated groups compared to vehicle are plotted. Significant difference from the vehicle treated group was calculated via One-way ANOVA using Graphpad Prism, *p<0.05, **p<0.01, ***p<0.001, ****p<0.0001.

FIG. 4E shows that MALT1 inhibition resulted in a dose dependent reduction in antigen specific autoantibody (α-collagen IgG) level while sparing total IgG antibody titers. Statistical analysis performed using one-way ANOVA (*p<0.05, **p<0.01).

FIGS. 5A and 5B show that MALT1i induced reduction of splenic Tregs are significantly more sensitive in healthy animals compared to diseased. Specifically, FIG. 5A shows that naïve, healthy animals were treated with MALT1i in parallel with the animals in the rat CIA study for two weeks. Frequencies of splenic Tregs were compared between healthy (H) and diseased (D) animals by flow cytometry (n=8/group). Significance between similar dosing groups in healthy and diseased animals was calculated using Mann Whitney Test using Graphpad Prism software, **p<0.01, ***p<0.001.

FIG. 6A depicts pharmacokinetics of the REO-981 MALT1 inhibitor in a rat collagen induced arthritis (CIA) model over a 24 h time period following dosing.

FIG. 6B depicts endpoint clinical score at various doses of the REO-981 MALT1 inhibitor in a rat collagen induced arthritis (CIA) model.

FIG. 6C depicts levels of Tregs following administration of REO-981 MALT1 inhibitor in a rat collagen induced arthritis (CIA) model. Frequency of splenic Tregs was significantly reduced at the highest dose of MALT1 inhibition.

FIG. 6D depicts clinical score and percent reduction in Tregs following administration of REO-981 MALT1 inhibitor with relation to compound exposure (PK) expressed as area under the curve (AUC) for a given dose. Plasma MALT1i concentrations expressed as AUC₀₋₂₄ₕ (x-axis) is plotted against corresponding clinical scores (percent reduction vs. vehicle) and frequency of splenic Tregs (percent reduction vs. naïve) (y-axis). Curve-fitting for AUC/clinical score and AUC/Treg relationships were performed using Graphpad Prism to show dose-related uncoupling of efficacy and Treg reduction.

FIG. 6E shows compiled exposure response data from 4 distinct MALT1 inhibitors show consistent dose-related uncoupling of efficacy and Treg reduction. Statistical analyses performed using one-way ANOVA (***p<0.001).

FIG. 6F depicts clinical score and percent reduction in Tregs following administration of REO-981 MALT1 inhibitor as a function of Cmax.

FIG. 6G depicts clinical score and percent reduction in Tregs following administration of REO-981 MALT1 inhibitor as a function of Ctrough (trough concentration).

FIG. 7A depicts pharmacokinetics of the REO-528 MALT1 inhibitor in a rat collagen induced arthritis (CIA) model over a 24 h time period following dosing.

FIG. 7B depicts endpoint clinical score at various doses of the REO-528 MALT1 inhibitor in a rat collagen induced arthritis (CIA) model.

FIG. 7C depicts levels of Tregs following administration of REO-528 MALT1 inhibitor in a rat collagen induced arthritis (CIA) model.

FIG. 7D depicts clinical score and percent reduction in Tregs following administration of REO-528 MALT1 inhibitor with relation to compound exposure (PK) expressed as area under the curve (AUC) for a given dose.

FIG. 7E depicts clinical score and percent reduction in Tregs following administration of REO-528 MALT1 inhibitor as a function of Cmax.

FIG. 7F depicts clinical score and percent reduction in Tregs following administration of REO-528 MALT1 inhibitor as a function of Ctrough (trough concentration).

FIG. 8A depicts pharmacokinetics of the REO-538 MALT1 inhibitor in a rat collagen induced arthritis (CIA) model over a 24 h time period following dosing.

FIG. 8B depicts endpoint clinical score at various doses of the REO-538 MALT1 inhibitor in a rat collagen induced arthritis (CIA) model.

FIG. 8C depicts levels of Tregs following administration of REO-538 MALT1 inhibitor in a rat collagen induced arthritis (CIA) model.

FIG. 8D depicts clinical score and percent reduction in Tregs following administration of REO-538 MALT1 inhibitor as a function of compound exposure expressed as area under the curve (AUC).

FIG. 9A depicts pharmacokinetics of the REO-703 MALT1 inhibitor in a rat collagen induced arthritis (CIA) model over a 24 h time period following dosing.

FIG. 9B depicts endpoint clinical score at various doses of the REO-703 MALT1 inhibitor in a rat collagen induced arthritis (CIA) model.

FIG. 9C depicts levels of Tregs following administration of REO-703 MALT1 inhibitor in a rat collagen induced arthritis (CIA) model.

FIG. 9D depicts clinical score and percent reduction in Tregs following administration of REO-703 MALT1 inhibitor as a function of compound exposure expressed as area under the curve (AUC).

FIG. 10A depicts pharmacokinetics of the REO-076 MALT1 inhibitor in a rat collagen induced arthritis (CIA) model over a 24 h time period following dosing.

FIG. 10B depicts endpoint clinical score at various doses of the REO-076 MALT1 inhibitor in a rat collagen induced arthritis (CIA) model.

FIG. 10C depicts levels of Tregs following administration of REO-076 MALT1 inhibitor in a rat collagen induced arthritis (CIA) model.

FIG. 10D depicts clinical score and percent reduction in Tregs following administration of REO-076 MALT1 inhibitor as a function of compound exposure expressed as area under the curve (AUC).

FIG. 11 shows qualitative characterization of compounds relative to efficacy and Treg impact of each of the various MALT1 inhibitors as observed in a rat collagen induced arthritis (CIA) model.

FIG. 12A shows dose-dependent efficacy of REO-528 and REO-703 MALT1 inhibitors in a rat CIA model.

FIG. 12B shows levels of Tregs following administration of REO-528 and REO-703 MALT1 inhibitors in a rat CIA model.

FIG. 12C shows plasma pharmacokinetics of REO-528.

FIG. 12D shows plasma pharmacokinetics of REO-703.

FIG. 13A shows endpoint clinical score across different dosing regimen involving REO-538 MALT inhibitor.

FIG. 13B shows pharmacokinetics of REO-538 over a 24 h time period following dosing across different dosing regimen.

FIG. 13C shows levels of IL-1β, IL-6, KC/GRO, and TNFα in synovial fluid following administration of REO-538.

FIG. 14 shows clinical score and percent reduction in Tregs following administration of REO-528 MALT1 inhibitor as a function of area under the curve (AUC).

FIG. 15 shows administration strategy for REO-528 MALT1 inhibitor, including combination REO-528+IL-2 therapy.

FIG. 16A shows percent suppression of naïve CD4⁺ T-cells by human Tregs co-cultured at varying ratios of Tregs to naïve CD4⁺ T-cells in the presence of indicated concentrations of MALT1i.

FIG. 16B is a histogram representation of FoxP3 MFI from nTregs stimulated with Dynabeads.

FIG. 16C shows CTV traces of naïve CD4⁺ T-cells activated with dynabeads for 3 days in culture in the presence of 1, 0.3, and 0.01 μM of MALT1i.

FIG. 16D is a quantitation of percent proliferating naïve CD4⁺ T cells from FIG. 16C. Data is representative of 2 independent experiments with 2 donors and 2 technical replicates per donor.

FIGS. 16E and 16F shows levels of pSTAT5 (Y694) measured from Tregs pre-treated with indicated concentrations of MALT1i or 1 μM Tofacitinib, followed by addition of 25 IU IL-2. Data is representative of 2 independent experiments with 2 donors and 2 technical replicates per donor.

FIG. 17A depicts Treg levels following administration of MALT1 inhibitors in naïve mice.

FIG. 17B shows pharmacokinetics profile of MALT1 inhibitors on Day 28.

FIG. 17C shows IC50 and Kd values of MALT1 inhibitors.

FIG. 18A depicts Treg levels following administration of REO-981 MALT1 inhibitor in a rat collagen induced arthritis (CIA) model or in healthy animals.

FIG. 18B depicts endpoint clinical scores following administration of REO-981 MALT1 inhibitor in a rat collagen induced arthritis (CIA) model.

FIG. 18C depicts the pharmacokinetics (PK) profile following administration of REO-981 MALT1 inhibitor in a rat collagen induced arthritis (CIA) model.

FIG. 18D depicts clinical score and percent reduction in Tregs following administration of REO-981 MALT1 inhibitor as a function of compound exposure expressed as area under the curve (AUC). Plasma MALT1i concentrations expressed as AUC₀₋₂₄ₕ (x-axis) is plotted against corresponding clinical scores (percent reduction vs. vehicle) and frequency of splenic Tregs (percent reduction vs. naïve) (y-axis). Curve-fitting for AUC/clinical score and AUC/Treg relationships were performed using Graphpad Prism to show dose-related uncoupling of efficacy and Treg reduction.

FIG. 19 shows compiled exposure response data from 4 distinct MALT1 inhibitors show consistent dose-related uncoupling of efficacy and Treg reduction. Statistical analyses performed using one-way ANOVA (***p<0.001).

FIG. 20A depicts average clinical score following administration of MALT1 inhibitors in a prophylactic experimental autoimmune encephalomyelitis (EAE) model.

FIG. 20B depicts the pharmacokinetics (PK) profile following administration of MALT1 inhibitors in a prophylactic experimental autoimmune encephalomyelitis (EAE) model.

FIG. 20C depicts average clinical score following administration of MALT1 inhibitors in a therapeutic experimental autoimmune encephalomyelitis (EAE) model.

FIG. 20D depicts the pharmacokinetics (PK) profile following administration of MALT1 inhibitors in a therapeutic experimental autoimmune encephalomyelitis (EAE) model.

FIG. 21A depicts the average GVHD score following administration of MALT1 inhibitors in a mouse GVHD model.

FIG. 21B depicts the pharmacokinetics (PK) profile following administration of MALT1 inhibitors in a mouse GVHD model.

FIGS. 22A and 22B show overall survival (OS) and progression-free survival (PFS) Kaplan Meier curves, respectively, in a murine model of sclerodermatous GVHD (scGVHD) following administration of a MALT1 inhibitor.

FIGS. 22C-22E shows levels of T follicular helper cells (T_{FH}) cells, Germinal Center (GC) B cells, and Treg cells in each of the different treatment groups.

FIG. 22F shows percentage change in ear thickness as a measure of delayed-type hypersensitivity following administration of a MALT1 inhibitor.

FIG. 23 depicts Treg levels following administration of MALT1 inhibitors in a mouse accelerated Lupus model.

### DETAILED DESCRIPTION

### Definitions

As used herein, the term “about” refers to a value that is within 10% above or below the value being described. For example, the term “about 5 nM” indicates a range of from 4.5 nM to 5.5 nM.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject. A sample may be, for example, withdrawn blood from a subject e.g., for determining levels of one or more biomarkers for determining whether the subject is a candidate subject.

As used herein, “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

### Methods of Treating Diseases or Disorders

The present invention is based, in part, on the discovery that a MALT1 inhibitor can be administered to a subject suffering from a disease (e.g., an autoimmune disease), wherein upon administration of MALT1 inhibitors, the efficacy of the MALT1 inhibitor against the disease is decoupled from the depleting effects on Treg cells.

In particular embodiments, efficacy of the MALT1 inhibitor against the disease (e.g., autoimmune disease) is measured according to an endpoint clinical score. An example of an endpoint clinical score is the Clinical Disease Activity Index (CDAI), which is a useful clinical composite score for following patients with rheumatoid arthritis. The CDAI is the sum of 4 outcome parameters: tender and swollen joint counts (28 joints assessed) and patient's and physician's global assessments of disease activity (on a 0-10-cm visual analog scale). Range of possible scores is 0-17. (Source: American College of Rheumatology). An additional example of an endpoint clinical score is a CIA clinical score which measures erythema and swollen joints which approximate swollen joint counts of the human composite score, CDAI.

In particular embodiments, efficacy of the MALT1 inhibitor against the disease (e.g., autoimmune disease) is measured according to a change in an endpoint clinical score. In particular embodiments, the reduction of Treg cells is measured according to the level of Treg cells following administration of the MALT1 inhibitor in comparison to the level of Treg cells before administration of the MALT1 inhibitor.

In various embodiments, the efficacy of the MALT1 inhibitor against the disease is decoupled from the depleting effects on Treg cells if the administration of the MALT1 inhibitor results in greater than a 30% change (e.g., increase or reduction) in the clinical score and less than a 50% reduction in Treg levels. In various embodiments, the efficacy of the MALT1 inhibitor against the disease is decoupled from the depleting effects on Treg cells if the administration of the MALT1 inhibitor results in greater than a 30% change (e.g., increase or reduction) in the clinical score and less than a 40% reduction in Treg levels. In various embodiments, the efficacy of the MALT1 inhibitor against the disease is decoupled from the depleting effects on Treg cells if the administration of the MALT1 inhibitor results in greater than a 30% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 30% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 30% change (e.g., increase or reduction) in the clinical score and less than a 25% reduction in Treg levels. In various embodiments, the efficacy of the MALT1 inhibitor against the disease is decoupled from the depleting effects on Treg cells if the administration of the MALT1 inhibitor results in greater than a 30% change (e.g., increase or reduction) in the clinical score and less than a 20% reduction in Treg levels. In various embodiments, the efficacy of the MALT1 inhibitor against the disease is decoupled from the depleting effects on Treg cells if the administration of the MALT1 inhibitor results in greater than a 30% change (e.g., increase or reduction) in the clinical score and less than a 10% reduction in Treg levels. In various embodiments, the efficacy of the MALT1 inhibitor against the disease is decoupled from the depleting effects on Treg cells if the administration of the MALT1 inhibitor results in greater than a 30% change (e.g., increase or reduction) in the clinical score and less than a 5% reduction in Treg levels.

In various embodiments, the efficacy of the MALT1 inhibitor against the disease is decoupled from the depleting effects on Treg cells if the administration of the MALT1 inhibitor results in greater than a 40% change (e.g., increase or reduction) in the clinical score and less than a 50% reduction in Treg levels. In various embodiments, the efficacy of the MALT1 inhibitor against the disease is decoupled from the depleting effects on Treg cells if the administration of the MALT1 inhibitor results in greater than a 40% change (e.g., increase or reduction) in the clinical score and less than a 40% reduction in Treg levels. In various embodiments, the efficacy of the MALT1 inhibitor against the disease is decoupled from the depleting effects on Treg cells if the administration of the MALT1 inhibitor results in greater than a 40% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 40% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 40% change (e.g., increase or reduction) in the clinical score and less than a 25% reduction in Treg levels. In various embodiments, the efficacy of the MALT1 inhibitor against the disease is decoupled from the depleting effects on Treg cells if the administration of the MALT1 inhibitor results in greater than a 40% change (e.g., increase or reduction) in the clinical score and less than a 20% reduction in Treg levels. In various embodiments, the efficacy of the MALT1 inhibitor against the disease is decoupled from the depleting effects on Treg cells if the administration of the MALT1 inhibitor results in greater than a 40% change (e.g., increase or reduction) in the clinical score and less than a 10% reduction in Treg levels. In various embodiments, the efficacy of the MALT1 inhibitor against the disease is decoupled from the depleting effects on Treg cells if the administration of the MALT1 inhibitor results in greater than a 40% change (e.g., increase or reduction) in the clinical score and less than a 5% reduction in Treg levels.

In various embodiments, the efficacy of the MALT1 inhibitor against the disease is decoupled from the depleting effects on Treg cells if the administration of the MALT1 inhibitor results in greater than a 50% change (e.g., increase or reduction) in the clinical score and less than a 50% reduction in Treg levels. In various embodiments, the efficacy of the MALT1 inhibitor against the disease is decoupled from the depleting effects on Treg cells if the administration of the MALT1 inhibitor results in greater than a 50% change (e.g., increase or reduction) in the clinical score and less than a 40% reduction in Treg levels. In various embodiments, the efficacy of the MALT1 inhibitor against the disease is decoupled from the depleting effects on Treg cells if the administration of the MALT1 inhibitor results in greater than a 50% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 50% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 50% change (e.g., increase or reduction) in the clinical score and less than a 25% reduction in Treg levels. In various embodiments, the efficacy of the MALT1 inhibitor against the disease is decoupled from the depleting effects on Treg cells if the administration of the MALT1 inhibitor results in greater than a 50% change (e.g., increase or reduction) in the clinical score and less than a 20% reduction in Treg levels. In various embodiments, the efficacy of the MALT1 inhibitor against the disease is decoupled from the depleting effects on Treg cells if the administration of the MALT1 inhibitor results in greater than a 50% change (e.g., increase or reduction) in the clinical score and less than a 10% reduction in Treg levels. In various embodiments, the efficacy of the MALT1 inhibitor against the disease is decoupled from the depleting effects on Treg cells if the administration of the MALT1 inhibitor results in greater than a 50% change (e.g., increase or reduction) in the clinical score and less than a 5% reduction in Treg levels.

In various embodiments, the efficacy of the MALT1 inhibitor against the disease is decoupled from the depleting effects on Treg cells if the administration of the MALT1 inhibitor results in greater than a 60% change (e.g., increase or reduction) in the clinical score and less than a 50% reduction in Treg levels. In various embodiments, the efficacy of the MALT1 inhibitor against the disease is decoupled from the depleting effects on Treg cells if the administration of the MALT1 inhibitor results in greater than a 60% change (e.g., increase or reduction) in the clinical score and less than a 40% reduction in Treg levels. In various embodiments, the efficacy of the MALT1 inhibitor against the disease is decoupled from the depleting effects on Treg cells if the administration of the MALT1 inhibitor results in greater than a 60% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 60% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 60% change (e.g., increase or reduction) in the clinical score and less than a 25% reduction in Treg levels. In various embodiments, the efficacy of the MALT1 inhibitor against the disease is decoupled from the depleting effects on Treg cells if the administration of the MALT1 inhibitor results in greater than a 60% change (e.g., increase or reduction) in the clinical score and less than a 20% reduction in Treg levels. In various embodiments, the efficacy of the MALT1 inhibitor against the disease is decoupled from the depleting effects on Treg cells if the administration of the MALT1 inhibitor results in greater than a 60% change (e.g., increase or reduction) in the clinical score and less than a 10% reduction in Treg levels. In various embodiments, the efficacy of the MALT1 inhibitor against the disease is decoupled from the depleting effects on Treg cells if the administration of the MALT1 inhibitor results in greater than a 60% change (e.g., increase or reduction) in the clinical score and less than a 5% reduction in Treg levels.

In various embodiments, the efficacy of the MALT1 inhibitor against the disease is decoupled from the depleting effects on Treg cells if the administration of the MALT1 inhibitor results in greater than a 70% change (e.g., increase or reduction) in the clinical score and less than a 50% reduction in Treg levels. In various embodiments, the efficacy of the MALT1 inhibitor against the disease is decoupled from the depleting effects on Treg cells if the administration of the MALT1 inhibitor results in greater than a 70% change (e.g., increase or reduction) in the clinical score and less than a 40% reduction in Treg levels. In various embodiments, the efficacy of the MALT1 inhibitor against the disease is decoupled from the depleting effects on Treg cells if the administration of the MALT1 inhibitor results in greater than a 70% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 70% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 70% change (e.g., increase or reduction) in the clinical score and less than a 25% reduction in Treg levels. In various embodiments, the efficacy of the MALT1 inhibitor against the disease is decoupled from the depleting effects on Treg cells if the administration of the MALT1 inhibitor results in greater than a 70% change (e.g., increase or reduction) in the clinical score and less than a 20% reduction in Treg levels. In various embodiments, the efficacy of the MALT1 inhibitor against the disease is decoupled from the depleting effects on Treg cells if the administration of the MALT1 inhibitor results in greater than a 70% change (e.g., increase or reduction) in the clinical score and less than a 10% reduction in Treg levels. In various embodiments, the efficacy of the MALT1 inhibitor against the disease is decoupled from the depleting effects on Treg cells if the administration of the MALT1 inhibitor results in greater than a 70% change (e.g., increase or reduction) in the clinical score and less than a 5% reduction in Treg levels.

In various embodiments, the efficacy of the MALT1 inhibitor against the disease is decoupled from the depleting effects on Treg cells if the administration of the MALT1 inhibitor results in greater than a 80% change (e.g., increase or reduction) in the clinical score and less than a 50% reduction in Treg levels. In various embodiments, the efficacy of the MALT1 inhibitor against the disease is decoupled from the depleting effects on Treg cells if the administration of the MALT1 inhibitor results in greater than a 80% change (e.g., increase or reduction) in the clinical score and less than a 40% reduction in Treg levels. In various embodiments, the efficacy of the MALT1 inhibitor against the disease is decoupled from the depleting effects on Treg cells if the administration of the MALT1 inhibitor results in greater than a 80% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 80% change (e.g., increase or reduction) in the clinical score and less than a 30% reduction in Treg levels. In various embodiments, the efficacy of the MALT1 inhibitor against the disease is decoupled from the depleting effects on Treg cells if the administration of the MALT1 inhibitor results in greater than a 80% change (e.g., increase or reduction) in the clinical score and less than a 25% reduction in Treg levels. In various embodiments, the efficacy of the MALT1 inhibitor against the disease is decoupled from the depleting effects on Treg cells if the administration of the MALT1 inhibitor results in greater than a 80% change (e.g., increase or reduction) in the clinical score and less than a 20% reduction in Treg levels. In various embodiments, the efficacy of the MALT1 inhibitor against the disease is decoupled from the depleting effects on Treg cells if the administration of the MALT1 inhibitor results in greater than a 80% change (e.g., increase or reduction) in the clinical score and less than a 10% reduction in Treg levels. In various embodiments, the efficacy of the MALT1 inhibitor against the disease is decoupled from the depleting effects on Treg cells if the administration of the MALT1 inhibitor results in greater than a 80% change (e.g., increase or reduction) in the clinical score and less than a 5% reduction in Treg levels.

In various embodiments, the efficacy of the MALT1 inhibitor against the disease is decoupled from the depleting effects on Treg cells based on the administration or dosing of the MALT1 inhibitor. In various embodiments, the MALT1 inhibitor is dosed to the subject at any of 1 mg/kg, 3 mg/kg, 10 mg/kg, 30 mg/kg, or 100 mg/kg such that the efficacy of the MALT1 inhibitor against the disease is decoupled from the depleting effects on Treg cells. In various embodiments, the MALT1 inhibitor is administered to the subject daily for about 14 days, after which the MALT1 inhibitor is withheld from the subject for about 7 days. In such embodiments, the on/off administration of the MALT1 inhibitor decouples the efficacy of the MALT1 inhibitor against the disease from the depleting effects on Treg cells.

In various embodiments, following administration of the MALT1 inhibitor to the subject, the MALT1 inhibitor achieves a particular pharmacokinetics (PK) profile that decouples the efficacy of the MALT1 inhibitor against the disease from the depleting effects on Treg cells. In various embodiments, following administration of the MALT1 inhibitor to the subject, the MALT1 inhibitor achieves a time over a IC50 blood concentration target from about 12 hours to about 24 hours per 24 hours. In various embodiments, following administration of the MALT1 inhibitor to the subject, the MALT1 inhibitor achieves a time over a IC90 blood concentration target from about 3 hours to about 12 hours per 24 hours. The time over IC50 and time over IC90 blood concentration target levels are described in further detail herein. In various embodiments, the time over a IC50 blood concentration target and/or the time over a IC90 blood concentration target decouples the efficacy of the MALT1 inhibitor against the disease from the depleting effects on Treg cells.

In various embodiments, following administration of the MALT1 inhibitor to the subject, the MALT1 inhibitor achieves a log₁₀(AUC) value from about 0.5 μg*hr/mL to about 2.0 μg*hr/mL. In various embodiments, following administration of the MALT1 inhibitor to the subject, the MALT1 inhibitor achieves a log₁₀(Cmax) value between 2.0 and 4.0 ng/ml. In such embodiments, the log₁₀(AUC) or log₁₀(Cmax) achieved by the MALT1 inhibitor following administration decouples the efficacy of the MALT1 inhibitor against the disease from the depleting effects on Treg cells.

### Administration and Dosing of MALT1 Inhibitors

Contemplated MALT1 inhibitors, may be administered to a subject, such as a mammalian subject (e.g., a human subject), by one or more routes of administration. In various embodiments, contemplated MALT1 inhibitors may be administered to a subject by intravenous, intraperitoneal, intramuscular, intraarterial, or subcutaneous infusion, among others. In various embodiments, contemplated MALT1 inhibitors may be administered to a subject via local administration. For example, for a brain-related diseases (e.g., multiple sclerosis), contemplated MALT1 inhibitors may be administered intracranially, intracerebrally, or intraventricularly. For example, for a brain-related diseases (e.g., multiple sclerosis), contemplated MALT1 inhibitors may be administered intracranially, intracerebrally, or intraventricularly. For example, for skin or joint related diseases (e.g., psoriatic arthritis, psoriasis, Lupus, or rheumatoid arthritis), contemplated MALT1 inhibitors may be locally administered (e.g., topically administered to the skin or locally injected into joints).

In some embodiments, the MALT1 inhibitor (e.g., a MALT1 inhibitor disclosed herein such as in Table 1A or Table 1B) is administered at a dose of from about 0.1 mg/kg to about 100 mg/kg, such as a dose of about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 21 mg/kg, 22 mg/kg, 23 mg/kg, 24 mg/kg, 25 mg/kg, 26 mg/kg, 27 mg/kg, 28 mg/kg, 29 mg/kg, 30 mg/kg, 31 mg/kg, 32 mg/kg, 33 mg/kg, 34 mg/kg, 35 mg/kg, 36 mg/kg, 37 mg/kg, 38 mg/kg, 39 mg/kg, 40 mg/kg, 41 mg/kg, 42 mg/kg, 43 mg/kg, 44 mg/kg, 45 mg/kg, 46 mg/kg, 47 mg/kg, 48 mg/kg, 49 mg/kg, 50 mg/kg, 51 mg/kg, 52 mg/kg, 53 mg/kg, 54 mg/kg, 55 mg/kg, 56 mg/kg, 57 mg/kg, 58 mg/kg, 59 mg/kg, 60 mg/kg, 61 mg/kg, 62 mg/kg, 63 mg/kg, 64 mg/kg, 65 mg/kg, 66 mg/kg, 67 mg/kg, 68 mg/kg, 69 mg/kg, 70 mg/kg, 71 mg/kg, 72 mg/kg, 73 mg/kg, 74 mg/kg, 75 mg/kg, 76 mg/kg, 77 mg/kg, 78 mg/kg, 79 mg/kg, 80 mg/kg, 81 mg/kg, 82 mg/kg, 83 mg/kg, 84 mg/kg, 85 mg/kg, 86 mg/kg, 87 mg/kg, 88 mg/kg, 89 mg/kg, 90 mg/kg, 91 mg/kg, 92 mg/kg, 93 mg/kg, 94 mg/kg, 95 mg/kg, 96 mg/kg, 97 mg/kg, 98 mg/kg, 99 mg/kg, or 100 mg/kg.

In various embodiments, the MALT1 inhibitor administered at a dose from about 1 mg/kg to about 6 mg/kg. In various embodiments, the MALT1 inhibitor is administered at a dose from about 2 mg/kg to about 5 mg/kg. In various embodiments, at a dose of about 3 mg/kg. In various embodiments, the MALT1 inhibitor is administered at a dose from about 8 mg/kg to about 20 mg/kg. In various embodiments, the MALT1 inhibitor is administered at a dose from about 9 mg/kg to about 15 mg/kg. In various embodiments, the MALT1 inhibitor is administered at a dose of about 10 mg/kg. In various embodiments, the MALT1 inhibitor is administered at a dose from about 8 mg/kg to about 20 mg/kg. In various embodiments, the MALT1 inhibitor is administered at a dose from about 9 mg/kg to about 15 mg/kg. In various embodiments, the MALT1 inhibitor is administered at a dose of about 10 mg/kg. In various embodiments, the MALT1 inhibitor is administered at a dose from about 20 mg/kg to about 40 mg/kg. In various embodiments, the MALT1 inhibitor is administered at a dose between from about 25 mg/kg to about 35 mg/kg. In various embodiments, the MALT1 inhibitor is administered at a dose of about 30 mg/kg. In various embodiments, the MALT1 inhibitor is administered at a dose between from about 50 mg/kg to about 100 mg/kg. In various embodiments, the MALT1 inhibitor is administered at a dose between from about 60 mg/kg to about 100 mg/kg. In various embodiments, the MALT1 inhibitor is administered at a dose between from about 70 mg/kg to about 100 mg/kg. In various embodiments, the MALT1 inhibitor is administered at a dose between from about 80 mg/kg to about 100 mg/kg. In various embodiments, the MALT1 inhibitor is administered at a dose between from about 90 mg/kg to about 100 mg/kg. In various embodiments, the MALT1 inhibitor is administered at a dose of about 100 mg/kg.

In various embodiments, a MALT1 inhibitor (e.g., a MALT1 inhibitor disclosed herein such as in Table 1A or Table 1B) may be administered to a subject one or more times daily, weekly, monthly, or yearly, depending on such factors as, for instance, the subject's age, body weight, sex, the subject's diet, and the subject's excretion rate. In particular embodiments, the MALT1 inhibitor is administered daily. In various embodiments, the MALT1 inhibitor is administered daily for between 5 to 20 days. In various embodiments, the MALT1 inhibitor is administered daily for between 5 to 8 days. In various embodiments, the MALT1 inhibitor is administered daily for 7 days. In various embodiments, the MALT1 inhibitor is administered daily for between 10 to 15 days. In various embodiments, the MALT1 inhibitor is administered daily for 14 days.

In various embodiments, a MALT1 inhibitor (e.g., a MALT1 inhibitor disclosed herein such as in Table 1A or Table 1B) may be administered to a subject over one or more cycles. In various embodiments, a cycle can include at a first period of time in which the MALT1 inhibitor is administered to the subject, followed by a second period of time in which the MALT1 inhibitor is withheld from the subject. In various embodiments, the MALT1 inhibitor is administered for one or more cycles, wherein a cycle comprises: administering the MALT1 inhibitor once per day for between 1-2 weeks followed by a treatment that does not comprise MALT1 inhibitor for 1-2 weeks. In various embodiments, the MALT1 inhibitor is administered for one or more cycles, wherein a cycle comprises: administering the MALT1 inhibitor once per day for between 10 to 14 days followed by a treatment that does not comprise MALT1 inhibitor for 7 to 10 days. In various embodiments, the MALT1 inhibitor is administered for one or more cycles, wherein a cycle comprises: administering the MALT1 inhibitor once per day for between 12 to 14 days followed by a treatment that does not comprise MALT1 inhibitor for 7 to 9 days. In various embodiments, the MALT1 inhibitor is administered for one or more cycles, wherein a cycle comprises: administering the MALT1 inhibitor once per day for about 2 weeks followed by a treatment that does not comprise MALT1 inhibitor for about 1 week. In various embodiments, a treatment that does not comprise MALT1 inhibitor is no treatment.

### Combination Therapy

A MALT1 inhibitor described herein may be administered in combination with another agent or therapy. A subject to be administered a MALT1 inhibitor disclosed herein may have a disease, disorder, or condition, or a symptom thereof, that would benefit from treatment with another agent or therapy.

In some embodiments, the MALT1 inhibitor described herein may be administered as the sole active ingredient or in conjunction with, e.g., as an adjuvant to, other drugs e.g., immunosuppressive or immunomodulating agents or other anti-inflammatory agents, e.g., for the treatment or prevention of alio- or xenograft acute or chronic rejection or inflammatory or autoimmune disorders, or a chemotherapeutic agent, e.g., a malignant cell anti-proliferative agent. For example, MALT1 inhibitors disclosed herein may be used in combination with a calcineurin inhibitor, e.g., cyclosporin A or FK 506; a mTOR inhibitor, e.g., rapamycin, 40-0-(2-hydroxyethyl)-rapamycin, biolimus-7 or biolimus-9; an ascomycin having immunosuppressive properties, e.g., ABT-281, ASM981; corticosteroids; cyclophosphamide; azathioprene; methotrexate; leflunomide; mizoribine; mycophenolic acid or salt; mycophenolate mofetil; or IL-1 beta inhibitor.

In some embodiments, a MALT1 inhibitor described herein is combined with a co-agent which is a PI3K inhibitor.

In some embodiments, a MALT1 inhibitor described herein is combined with co-agent that influence BTK (Bruton's tyrosine kinase).

For the treatment of oncological diseases, a MALT1 inhibitor described herein may be used in combination with B-cell modulating agents, e.g., Rituximab, Ofatumumab, BTK or SYK inhibitors, inhibitors of PKC, PI3K, PDK, PIM, JAK and rmTOR and BH3 mimetics.

In various embodiments, a MALT1 inhibitor described herein may be administered in combination with one or more cytokines. In various embodiments, a MALT1 inhibitor described herein may be administered in combination with IL-2. In various embodiments, a MALT1 inhibitor described herein may be administered in combination with IL-15. In various embodiments, a MALT1 inhibitor described herein may be administered in combination with IL7.

In particular embodiments, a MALT1 inhibitor described herein may be administered in combination with IL-2, wherein the IL-2 is administered at a low dose to avoid toxicity. In various embodiments, a low dose of IL-2 is from about 10,000 International Units (IUs) to about 50,000 International Units (IUs). In particular embodiments, a MALT1 inhibitor described herein may be administered in combination with IL-2, wherein the IL-2 is administered at a dose from about 20,000 International Units (IUs) to about 40,000 International Units (IUs). In particular embodiments, a MALT1 inhibitor described herein may be administered in combination with IL-2, wherein the IL-2 is administered at a dose of about 30,000 International Units (IUs). In various embodiments, a low dose of IL-2 is from about 100,000 International Units (IUs) to about 5 million International Units (IUs). In various embodiments, a low dose of IL-2 is from about 500,000 International Units (IUs) to about 4.5 million International Units (IUs). In various embodiments, a low dose of IL-2 is from about 1 million International Units (IUs) to about 4 million International Units (IUs). In various embodiments, a low dose of IL-2 is from about 2 million International Units (IUs) to about 3 million International Units (IUs). In various embodiments, a low dose of IL-2 is about 3 million International Units (IUs).

In some embodiments, a MALT1 inhibitor described herein may be administered either simultaneously with, or before or after, one or more other therapeutic agent. In some embodiments, the MALT1 inhibitor described herein may be administered separately, by the same or different route of administration, or together in the same pharmaceutical composition as the other agents. In various embodiments, the MALT1 inhibitor is simultaneously administered to the subject with the second agent. In such embodiments, the MALT1 inhibitor may be co-formulated with the second agent. For example, the MALT1 inhibitor is combined with the second agent as a single pharmaceutical composition prior to administration to the subject.

In various embodiments, the MALT1 inhibitor is administered to the subject prior to administration of the second agent. In various embodiments, the MALT1 inhibitor is administered to the subject at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours prior to administration of the second agent. In various embodiments, the MALT1 inhibitor is administered to the subject at least 2 days, at least 3 days, at least 4 days, at least 5 days at least 6 days, at least 7 days, at least 8 days, at least 9 days, at least 10 days, at least 11 days, at least 12 days, at least 13 days, or at least 14 days prior to administration of the second agent.

In various embodiments, the MALT1 inhibitor is administered to the subject after administration of the second agent. In various embodiments, the MALT1 inhibitor is administered to the subject at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours after administration of the second agent. In various embodiments, the MALT1 inhibitor is administered to the subject at least 2 days, at least 3 days, at least 4 days, at least 5 days at least 6 days, at least 7 days, at least 8 days, at least 9 days, at least 10 days, at least 11 days, at least 12 days, at least 13 days, or at least 14 days prior to administration of the second agent.

### Characteristics Following Administration of MALT1 Inhibitors

Disclosed herein are methods for administering a MALT1 inhibitor to a subject, wherein following administration to the subject, the MALT1 inhibitor achieves particular characteristics in the subject. Example characteristics include a pharmacokinetics (PK) profile, a pharmacodynamics (PD) profile, level of Tregs, or percent reduction of Tregs.

In various embodiments, the MALT1 inhibitor achieves a particular pharmacokinetics (PK) or pharmacodynamics (PD) profile in the subject. In various embodiments, the PK profile or the PK profile in the subject enables the decoupling of the efficacy of the MALT1 inhibitor from its depletive effect on regulatory T cells (Tregs). In particular embodiments, the plasma PK profile of the MALT1 inhibitor enables the decoupling of the efficacy of the MALT1 inhibitor from its depletive effect on regulatory T cells (Tregs).

In various embodiments, the plasma PK profile of the MALT1 inhibitor refers to a time over an IC50 blood concentration target following administration to the subject. As used herein, a “time over an IC50 blood concentration target” refers to a period of time following administration in which a concentration of the MALT1 inhibitor in the subject's blood is above the IC50 value. As used herein, an IC50 value refers to a concentration of the compound (e.g., MALT1 inhibitor) resulting in 50% inhibition of the target-related process. In various embodiments, an IC50 blood concentration target of a MALT1 inhibitor is between 100 ng/ml and 1000 ng/mL. In various embodiments, an IC50 blood concentration target a MALT1 inhibitor is between 100 ng/ml and 500 ng/mL. In various embodiments, an IC50 blood concentration target a MALT1 inhibitor is between 100 ng/ml and 400 ng/ml. In various embodiments, an IC50 blood concentration target a MALT1 inhibitor is between 100 ng/mL and 300 ng/mL. In various embodiments, an IC50 blood concentration target a MALT1 inhibitor is between 100 ng/ml and 250 ng/mL. In various embodiments, an IC50 blood concentration target a MALT1 inhibitor is between 100 ng/ml and 200 ng/mL. In various embodiments, an IC50 blood concentration target a MALT1 inhibitor is between 200 ng/mL and 1000 ng/mL. In various embodiments, an IC50 blood concentration target a MALT1 inhibitor is between 200 ng/ml and 500 ng/mL. In various embodiments, an IC50 blood concentration target a MALT1 inhibitor is between 200 ng/mL and 400 ng/mL. In various embodiments, an IC50 blood concentration target a MALT1 inhibitor is between 200 ng/mL and 300 ng/mL. In various embodiments, an IC50 blood concentration target a MALT1 inhibitor is between 200 ng/ml and 250 ng/mL. In various embodiments, an IC50 blood concentration target a MALT1 inhibitor is between 300 ng/ml and 1000 ng/mL. In various embodiments, an IC50 blood concentration target a MALT1 inhibitor is between 300 ng/mL and 500 ng/mL. In various embodiments, an IC50 blood concentration target a MALT1 inhibitor is between 300 ng/ml and 400 ng/ml. In various embodiments, an IC50 blood concentration target a MALT1 inhibitor is between 400 ng/ml and 1000 ng/mL. In various embodiments, an IC50 blood concentration target a MALT1 inhibitor is between 400 ng/mL and 500 ng/mL. In various embodiments, an IC50 blood concentration target a MALT1 inhibitor is between 500 ng/ml and 1000 ng/ml.

In various embodiments, the plasma PK profile of the MALT1 inhibitor refers to a time over an IC90 blood concentration target following administration to the subject. As used herein, a “time over an IC90 blood concentration target” refers to a period of time following administration in which a concentration of the MALT1 inhibitor in the subject's blood is above the IC90 value. As used herein, an IC90 value refers to a concentration of the compound (e.g., MALT1 inhibitor) resulting in 90% inhibition of the target-related process. Generally, the IC90 value is considered as the concentration of the compound (e.g., MALT1 inhibitor) resulting in complete target coverage. In various embodiments, an IC90 blood concentration target of a MALT1 inhibitor is between 1000 ng/ml and 10000 ng/mL. In various embodiments, an IC90 blood concentration target a MALT1 inhibitor is between 1000 ng/mL and 5000 ng/mL. In various embodiments, an IC90 blood concentration target a MALT1 inhibitor is between 1000 ng/ml and 4000 ng/mL. In various embodiments, an IC90 blood concentration target a MALT1 inhibitor is between 1000 ng/mL and 3000 ng/mL. In various embodiments, an IC90 blood concentration target a MALT1 inhibitor is between 1000 ng/ml and 2500 ng/mL. In various embodiments, an IC90 blood concentration target a MALT1 inhibitor is between 1000 ng/ml and 2000 ng/mL. In various embodiments, an IC90 blood concentration target a MALT1 inhibitor is between 2000 ng/ml and 10000 ng/mL. In various embodiments, an IC90 blood concentration target a MALT1 inhibitor is between 2000 ng/ml and 5000 ng/mL. In various embodiments, an IC90 blood concentration target a MALT1 inhibitor is between 2000 ng/ml and 4000 ng/mL. In various embodiments, an IC90 blood concentration target a MALT1 inhibitor is between 2000 ng/mL and 3000 ng/mL. In various embodiments, an IC90 blood concentration target a MALT1 inhibitor is between 2000 ng/ml and 2500 ng/ml. In various embodiments, an IC90 blood concentration target a MALT1 inhibitor is between 3000 ng/ml and 10000 ng/mL. In various embodiments, an IC90 blood concentration target a MALT1 inhibitor is between 3000 ng/ml and 5000 ng/mL. In various embodiments, an IC90 blood concentration target a MALT1 inhibitor is between 3000 ng/mL and 4000 ng/mL. In various embodiments, an IC90 blood concentration target a MALT1 inhibitor is between 4000 ng/ml and 10000 ng/mL. In various embodiments, an IC90 blood concentration target a MALT1 inhibitor is between 4000 ng/mL and 5000 ng/mL. In various embodiments, an IC90 blood concentration target a MALT1 inhibitor is between 5000 ng/mL and 10000 ng/ml.

In various embodiments, following administration of the MALT1 inhibitor, the MALT1 inhibitor achieves a time over the IC50 blood concentration target from about 4 hours to about 20 hours per 24 hours. In various embodiments, following administration of the MALT1 inhibitor, the MALT1 inhibitor achieves a time over the IC50 blood concentration target from about 6 hours to about 18 hours per 24 hours. In various embodiments, following administration of the MALT1 inhibitor, the MALT1 inhibitor achieves a time over the IC50 blood concentration target from about 8 hours to about 16 hours per 24 hours. In various embodiments, following administration of the MALT1 inhibitor, the MALT1 inhibitor achieves a time over the IC50 blood concentration target from about 10 hours to about 14 hours per 24 hours. In various embodiments, following administration of the MALT1 inhibitor, the MALT1 inhibitor achieves a time over the IC50 blood concentration target from about 12 hours to about 24 hours per 24 hours. In various embodiments, following the administration to the subject, the MALT1 inhibitor achieves a time over the IC50 blood concentration target from about 16 hours to about 24 hours per 24 hours. In various embodiments, following the administration to the subject, the MALT1 inhibitor achieves a time over the IC50 blood concentration target from about 22 hours to about 24 hours per 24 hours.

In various embodiments, following the administration to the subject, the MALT1 inhibitor achieves a time over a IC90 blood concentration target from about 1 hour to about 15 hours per 24 hours. In various embodiments, following the administration to the subject, the MALT1 inhibitor achieves a time over a IC90 blood concentration target from about 3 hours to about 12 hours per 24 hours. In various embodiments, following the administration to the subject, the MALT1 inhibitor achieves a time over a IC90 blood concentration target from about 6 hours to about 10 hours per 24 hours. In various embodiments, following administration to the subject, the MALT1 inhibitor achieves a time over a IC90 blood concentration target from about 6 hours to about 24 hours per 24 hours. In various embodiments, following administration to the subject, the MALT1 inhibitor achieves a time over a IC90 blood concentration target from about 12 hours to about 24 hours per 24 hours. In various embodiments, following administration to the subject, the MALT1 inhibitor achieves a time over a IC90 blood concentration target from about 18 hours to about 24 hours per 24 hours. In various embodiments, following administration to the subject, the MALT1 inhibitor achieves a time over a IC90 blood concentration target from about 21 hours to about 24 hours per 24 hours.

Example IC50 and IC90 values of MALT1 inhibitors are shown below.

| | IC50 (mM) | IC90 (mM) |
|---|---|---|
| REO-1014981 (REO-981) | 0.3 | 2.0 |
| REO-1017528 (REO-528) | 0.82 | 5.78 |
| REO-1017538 (REO-538) | 1.09 | 10.4 |
| REO-1017703 (REO-703) | 0.38 | 4.95 |

In various embodiments, the plasma PK profile of the MALT1 inhibitor refers to an area under the curve (AUC) value following administration of the MALT1 inhibitor. Generally, the AUC value represents the exposure to the MALT1 inhibitor experienced by the subject. In various embodiments, following administration to the subject, the MALT1 inhibitor achieves a log₁₀(AUC) from about 0.5 μg*hr/mL to about 2.0 μg*hr/mL. In various embodiments, following administration to the subject, the MALT1 inhibitor achieves a log₁₀(AUC) from about 1.0 μg*hr/mL to about 1.75 μg*hr/mL. In various embodiments, following administration to the subject, the MALT1 inhibitor achieves a log₁₀(AUC) from about 1.25 μg*hr/mL to about 1.50 μg*hr/mL.

In various embodiments, the plasma PK profile of the MALT1 inhibitor refers to a Cmax value following administration of the MALT1 inhibitor. Generally, the Cmax value represents the maximum observed concentration of MALT1. In various embodiments, following administration to the subject, the MALT1 inhibitor achieves a log₁₀(Cmax) value between 2.0 and 4.0 ng/mL. In various embodiments, following administration to the subject, the MALT1 inhibitor achieves a log₁₀(Cmax) value between 2.0 and 2.5 ng/mL. In various embodiments, following administration to the subject, the MALT1 inhibitor achieves a log₁₀(Cmax) value between 2.5 and 3.0 ng/mL. In various embodiments, following administration to the subject, the MALT1 inhibitor achieves a log₁₀(Cmax) value between 3.0 and 3.5 ng/ml. In various embodiments, following administration to the subject, the MALT1 inhibitor achieves a log₁₀(Cmax) value between 3.0 and 4.0 ng/mL. In various embodiments, following administration to the subject, the MALT1 inhibitor achieves a log₁₀(Cmax) value between 3.5 and 4.0 ng/mL.

In various embodiments, following administration of the MALT1 inhibitor, the level of Tregs in the subject remains near the level of Tregs prior to administration of the MALT1 inhibitor. Thus, administration of the MALT1 inhibitor does not deplete the level of Tregs in the subject. n various embodiments, following administration of the MALT1 inhibitor to the subject, a level of Tregs of the subject remains at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or at least 100% of a level prior to administration. In various embodiments, following administration of the MALT1 inhibitor to the subject, a level of Tregs of the subject remains at least 60% of a level prior to administration. In various embodiments, following administration of the MALT1 inhibitor to the subject, a level of Tregs of the subject remains at least 70% of a level prior to administration. In various embodiments, following administration of the MALT1 inhibitor to the subject, a level of Tregs of the subject remains at least 80% of a level prior to administration. In various embodiments, following administration of the MALT1 inhibitor to the subject, a level of Tregs of the subject remains at least 90% of a level prior to administration. In various embodiments, following administration of the MALT1 inhibitor to the subject, a level of Tregs of the subject remains at least 95% of a level prior to administration. In various embodiments, following administration of the MALT1 inhibitor to the subject, a level of Tregs of the subject remains at least 99% of a level prior to administration.

### Methods of Identifying Candidate Subjects for Receiving MALT1 Inhibitors

Disclosed herein are methods for identifying candidate subjects selected for administration of a MALT1 inhibitor. Generally, candidate subjects represent subjects that are likely to respond favorably to a MALT inhibitor. For example, following administration of a MALT1 inhibitor in a candidate subject, the efficacy of the MALT1 inhibitor may be decoupled from its depletive effects on Treg cells. In various embodiments, a candidate subject who receives a MALT1 inhibitor may respond more favorably than a non-candidate subject. In various embodiments, a candidate subject who receives a MALT1 inhibitor experiences improved efficacy due to the MALT1 inhibitor in comparison to a non-candidate subject. In various embodiments, a candidate subject who receives a MALT1 inhibitor experiences reduced Treg reduction in comparison to a non-candidate subject.

In various embodiments, if administered in candidate subjects, a MALT1 inhibitor exhibits a larger range of log₁₀AUC values in which the efficacy and Treg reduction effects of the MALT1 inhibitor are decoupled in comparison to a corresponding range of log₁₀AUC values if the MALT1 inhibitor is administered in non-candidate subjects. In various embodiments, if administered in candidate subjects, a MALT1 inhibitor exhibits a larger range of log₁₀Cmax values in which the efficacy and Treg reduction effects of the MALT1 inhibitor are decoupled in comparison to a corresponding range of log₁₀Cmax values if the MALT1 inhibitor is administered in non-candidate subjects. In various embodiments, if administered in candidate subjects, a MALT1 inhibitor exhibits a larger range of log₁₀Ctrough values in which the efficacy and Treg reduction effects of the MALT1 inhibitor are decoupled in comparison to a corresponding range of log₁₀Ctrough values if the MALT1 inhibitor is administered in non-candidate subjects.

In various embodiments, a MALT1 inhibitor administered in candidate subjects exhibits at least a 10% increase in a range of log₁₀AUC values in which the efficacy and Treg reduction effects of the MALT1 inhibitor are decoupled in comparison to a corresponding range of log₁₀AUC values when the MALT1 inhibitor is administered in non-candidate subjects. In various embodiments, a MALT1 inhibitor administered in candidate subjects exhibits at least a 20% increase, at least a 30% increase, at least a 40% increase, at least a 50% increase, at least a 60% increase, at least a 70% increase, at least a 80% increase, at least a 90% increase, at least a 100% increase, at least a 110% increase, at least a 120% increase, at least a 130% increase, at least a 140% increase, at least a 150% increase, at least a 160% increase, at least a 170% increase, at least a 180% increase, at least a 190% increase, at least a 200% increase, at least a 300% increase, at least a 400% increase, or at least a 500% increase in a range of log₁₀AUC values in which the efficacy and Treg reduction effects of the MALT1 inhibitor are decoupled in comparison to a corresponding range of log₁₀AUC values when the MALT1 inhibitor is administered in non-candidate subjects. In particular embodiments, a MALT1 inhibitor administered in candidate subjects exhibits at least a 50% increase in a range of log₁₀AUC values in which the efficacy and Treg reduction effects of the MALT1 inhibitor are decoupled in comparison to a corresponding range of log₁₀AUC values when the MALT1 inhibitor is administered in non-candidate subjects. In various embodiments, a MALT1 inhibitor administered in candidate subjects exhibits at least a 100% increase in a range of log₁₀AUC values in which the efficacy and Treg reduction effects of the MALT1 inhibitor are decoupled in comparison to a corresponding range of log₁₀AUC values when the MALT1 inhibitor is administered in non-candidate subjects.

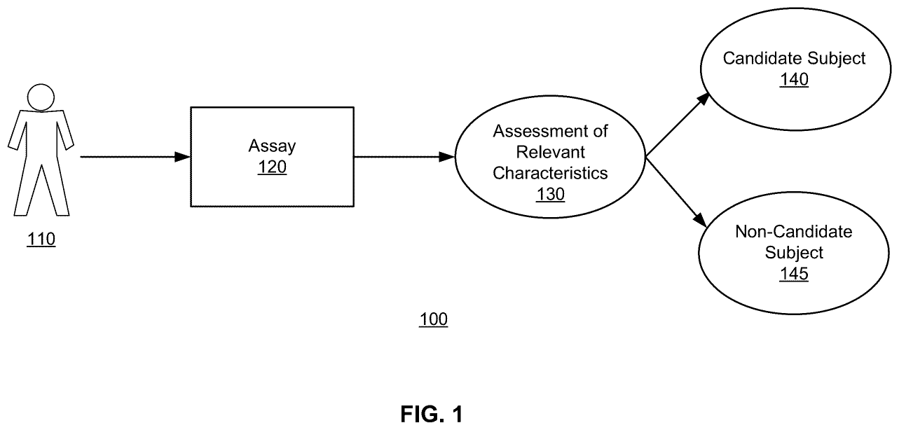

Reference is now made to FIG. 1, which depicts an example method 100 for identifying candidate subjects for receiving a MALT1 inhibitor, in accordance with an embodiment. FIG. 1 introduces a subject 110 who, by undergoing the method shown in FIG. 1, is categorized as either a candidate subject 140 or a non-candidate subject 145. In various embodiments, a assay 120 is performed on a sample obtained from the subject. As used herein, a “sample” or “test sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. The sample can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art.

In various embodiments, the sample is tested to determine values of one or more biomarkers by performing the assay 120. Here, the assay 120 may be a marker quantification assay that determines quantitative expression values of one or more biomarkers from the test sample. The assay 120 may be an immunoassay, and more specifically, a multi-plex immunoassay, examples of which are described in further detail below. The expression levels of various biomarkers can be obtained in a single run using a single test sample obtained from the subject 110. The quantified expression values of the biomarkers can then be assessed (e.g., assessment of relevant characteristics 130) to categorize the subject 110 as a candidate subject 140 or a non-candidate subject 145.

In various embodiments, the assay 120 for one or more biomarkers include DNA assays, microarrays, polymerase chain reaction (PCR), RT-PCR, Southern blots, Northern blots, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays. In various embodiments, the information from the assay 120 can be quantitative. In various embodiments, the information from the assay 120 can be qualitative, such as observing patterns or fluorescence, which can be translated into a quantitative measure by a user or automatically by a reader or computer system.

Various immunoassays designed to quantitate markers can be used in screening including multiplex assays. Measuring the concentration of a target marker in a sample or fraction thereof can be accomplished by a variety of specific assays. For example, a conventional sandwich type assay can be used in an array, ELISA, RIA, etc. format. Other immunoassays include Ouchterlony plates that provide a simple determination of antibody binding. Additionally, Western blots can be performed on protein gels or protein spots on filters, using a detection system specific for the markers as desired, conveniently using a labeling method.

Protein based analysis, using an antibody that specifically binds to a polypeptide (e.g. marker), can be used to quantify the marker level in a test sample obtained from a subject. In various embodiments, an antibody that binds to a marker can be a monoclonal antibody. In various embodiments, an antibody that binds to a marker can be a polyclonal antibody. For multiplex analysis of markers, arrays containing one or more marker affinity reagents, e.g. antibodies can be generated. Such an array can be constructed comprising antibodies against markers. Detection can utilize one or a panel of marker affinity reagents, e.g. a panel or cocktail of affinity reagents specific for one, two, three, four, five, six, seven, eight, nine, ten, eleven, twelve, thirteen, fourteen, fifteen, sixteen, seventeen, eighteen, nineteen, twenty, twenty one, or more markers.

In various embodiments, prior to implementation of an assay 120 (e.g., an immunoassay), a sample obtained from a subject 110 can be processed. In various embodiments, processing the sample enables the implementation of the assay 120 to more accurately evaluate expression levels of one or more biomarkers in the sample.

In various embodiments, the sample from a subject can be processed to extract biomarkers from the sample. In one embodiment, the sample can undergo phase separation to separate the biomarkers from other portions of the sample. For example, the sample can undergo centrifugation (e.g., pelleting or density gradient centrifugation) to separate larger and/or more dense entities in the sample (e.g., cells and other macromolecules) from the biomarkers. Other examples include filtration (e.g., ultrafiltration) to phase separate the biomarkers from other portions of the sample.

In various embodiments, the sample from a subject can be processed to produce a sub-sample with a fraction of biomarkers that were in the sample. In various embodiments, producing a fraction of biomarkers can involve performing a protein fractionation procedure. One example of protein fractionation procedures include chromatography (e.g., gel filtration, ion exchange, hydrophobic chromatography, or affinity chromatography). In particular embodiments, the protein fractionation procedure involves affinity purification or immunoprecipitation where biomarkers are bound by specific antibodies. Such antibodies can be immobilized on a support, such as a magnetic particle or nanoparticle or a plate.

In various embodiments, the sample from the subject 110 is processed to extract biomarkers from the sample and further processed to produce a sub-sample with a fraction of extracted biomarkers. Altogether, this enables a purified sub-sample of biomarkers that are of particular interest. Thus, implementing an assay (e.g., an immunoassay) for evaluating expression levels of the biomarkers of particular interest can be more accurate and of higher quality.

In various embodiments, the assay 120 determines quantitative expression values biomarkers comprising any of IL-2, IL-7, and IL-15.

In various embodiments, the assay 120 determines levels of one or more cells in the sample from the subject 110. In various embodiments, the assay 120 may determine levels of one or more immune cells in the sample from the subject 110. In particular embodiments, the assay 120 may determine levels of any one of lymphocytes, NK cells, Th17 cells, Treg cells, white blood cells, platelet counts, hemoglobin, erythrocyte counts, erythrocyte sedimentation rate. In various embodiments, the information from the assay 120 can be used to calculate a score for the subject 110, such as a disease activity score (DAS). Example methods for determining levels of any one of lymphocytes, NK cells, Th17 cells, Treg cells, white blood cells, platelet counts, hemoglobin, erythrocyte counts, erythrocyte sedimentation rate and/or for determining a disease activity score (DAS) is described in further detail in Li et al., “Increased Serum Interleukin-2 Levels Are Associated with Abnormal Peripheral Blood Natural Killer Cell Levels in Patients with Active Rheumatoid Arthritis.” Mediators of Inflammation, vol. 2020, Article ID 6108342, 15 pages, 2020, which is hereby incorporated by reference in its entirety.

Returning to FIG. 1, in various embodiments, step 130 involves assessing the relevant characteristics, including expression values of any one of the biomarkers (e.g., of IL-2, IL-15, and IL-7). Depending on the assessment at step 130, the subject is categorized as a candidate subject 140 or a non-candidate subject 145.

In particular embodiments, the subject 110 is determined to be a candidate subject 140 if the assessment identifies the subject as having elevated IL-2. In particular embodiments, the assessment identifies the subject as having elevated IL-2 if the subject has a level of IL-2 that is higher than a reference value. In various embodiments, the reference value represents an IL-2 value corresponding to a plurality of reference individuals, such as healthy individuals.

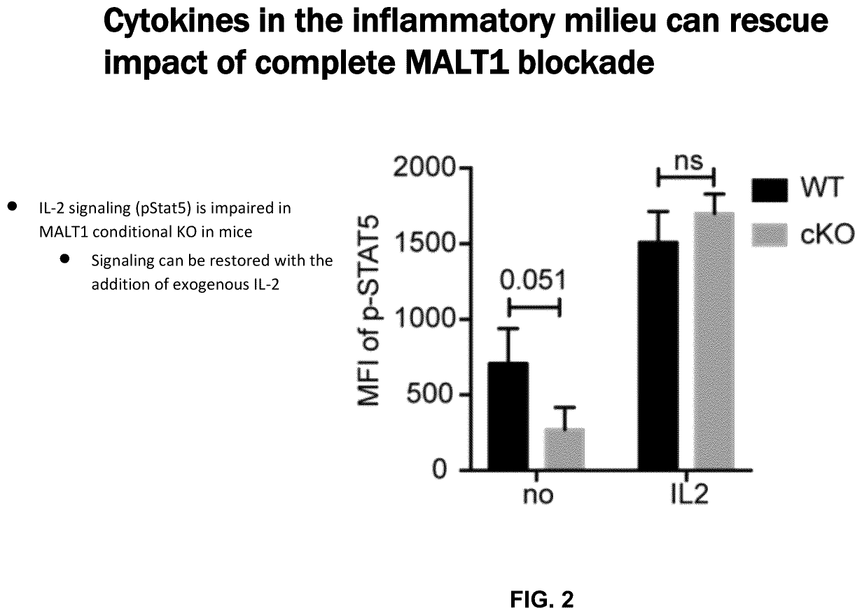

Reference is now made to FIG. 2, which depicts restoration of IL-2 signaling (pStat5) in MALT1 conditional knockout mice with the addition of exogenous IL-2. Further details of the restoration of IL-2 signaling in MALT1 conditional knockout mice in view of exogenous IL-2 is described in Cheng et al., MALT1 Protease is Critical in Maintaining Function of Regulatory T Cells and May be a Therapeutic Target for Antitumor Immunity,” J Immunol 2019; 202:3008-3019, which is hereby incorporated by reference in its entirety. Notably, IL-2 signaling (pStat5) is impaired in MALT1 conditional knockout in mice. MALT1 KO in mice may simulate the administration of a MALT1 inhibitor at a high concentration such that it depletes Treg levels. Furthermore, the addition of exogenous IL-2 restores the pStat5 signaling. Thus, this suggests that cytokines (e.g., IL-2) in the inflammatory milieu can rescue the impact of complete MALT1 blockade (e.g., caused by administration of high concentration of MALT1 inhibitor). Given that reduction of Treg levels is a result of MALT1 blockade, the presence or addition of IL-2 can reverse and rescue Treg reduction.

In particular embodiments, the subject 110 is determined to be a candidate subject 140 if the assessment identifies the subject as having elevated IL-7. In particular embodiments, the assessment identifies the subject as having elevated IL-7 if the subject has a level of IL-7 that is higher than a reference value. In various embodiments, the reference value represents an IL-7 value corresponding to a plurality of reference individuals, such as healthy individuals.

In particular embodiments, the subject 110 is determined to be a candidate subject 140 if the assessment identifies the subject as having an elevated IL-15. In particular embodiments, the assessment identifies the subject as having an elevated IL-15 if the subject has an elevated IL-15 that is higher than a reference value. In various embodiments, the reference value represents a IL-15 corresponding to a plurality of reference individuals, such as healthy individuals.

### MALT1 Inhibitors

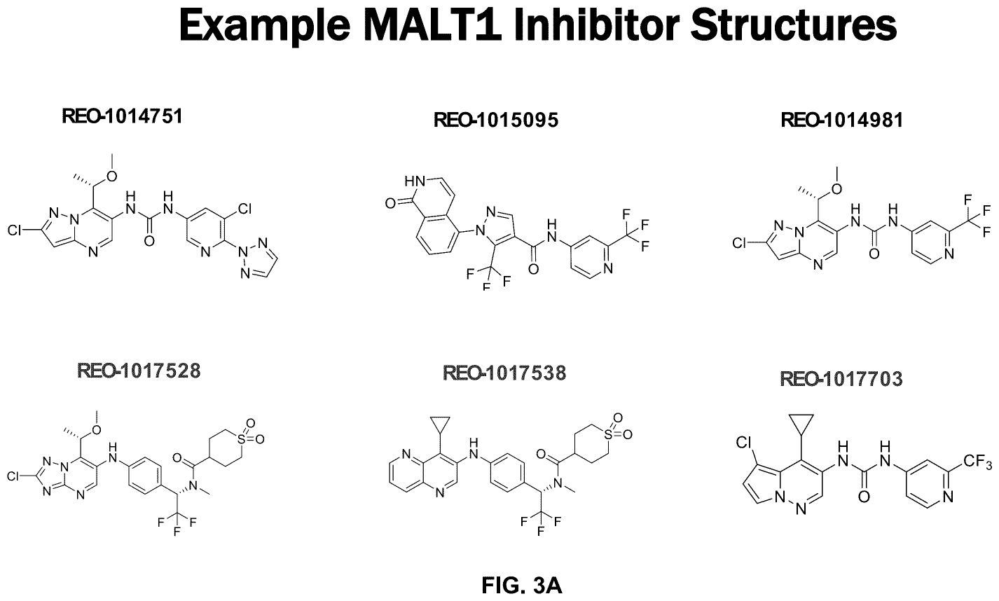

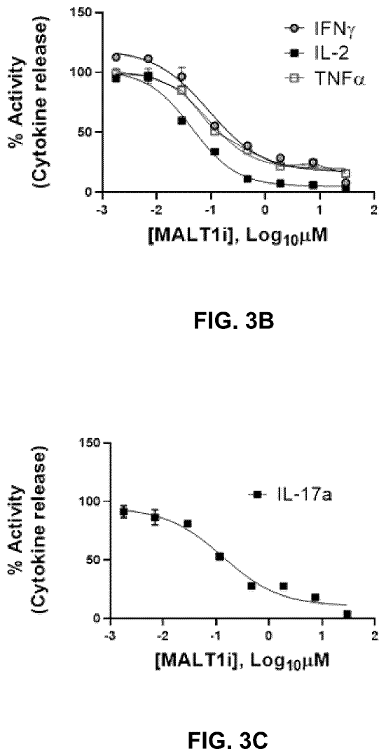

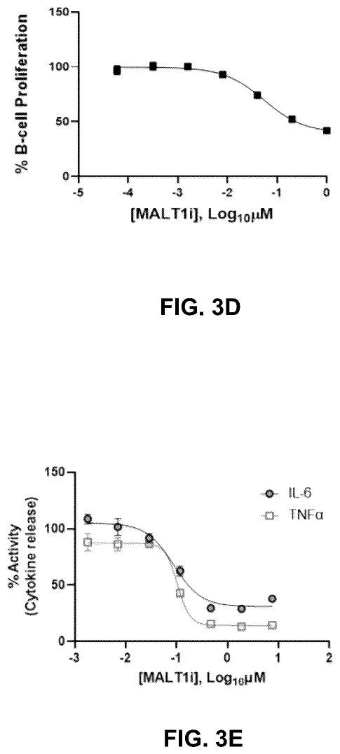

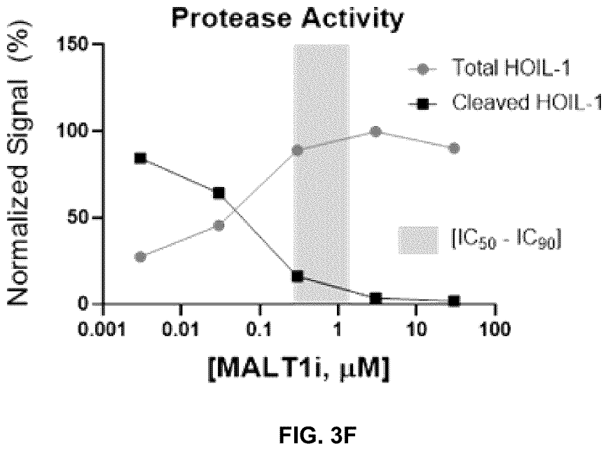

Disclosed herein are MALT1 inhibitors useful as therapeutic agents for treating diseases, such as autoimmune diseases. In particular, disclosed herein are methods for administering MALT1 inhibitors to a subject such that the efficacy of the MALT1 inhibitor against a disease is decoupled from Treg reduction. Exemplary MALT1 inhibitors are detailed in FIG. 3A, Table 1A, and/or Table 1B. In various embodiments, any of the MALT1 inhibitors shown in Table 1A or Table 1B can be administered to a subject. Additional example MALT1 inhibitors are described in WO2021138298 and WO2021207343, each of which is hereby incorporated by reference in its entirety. Further examples of MALT1 inhibitors include JNJ-67856633, CTX-177, and MLT-943. Additional example MALT1 inhibitors are described in WO2018119036, US20210332045, WO202134004, WO2020111087, WO2018020474, WO2017040304, WO2015181747, Fontan, L. et al, Chemically Induced Degradation of MALT1 to Treat B-Cell Lymphomas. Blood 2019; 134:2073, and Hamp, I. et al, A patent review of MALT1 inhibitors (2013-present), Expert Opinion on Therapeutic Patents, 31:12, (2021), 1079-1096, each of which is incorporated by reference in its entirety.

TABLE 1A
Example MALT1 inhibitors

| Compound Identifier | Compound Structure |
|---|---|
| REO-1014751 (REO-751) | 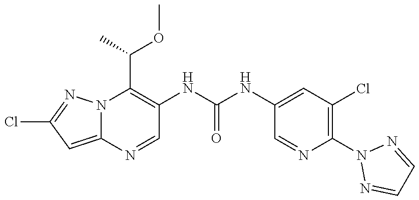 [SMILES: CO[C@@H](C)c1c(NC(=O)Nc2cnc(-n3nccn3)c(Cl)c2)cnc2cc(Cl)nn12] |
| REO-1015095 (REO-095) | 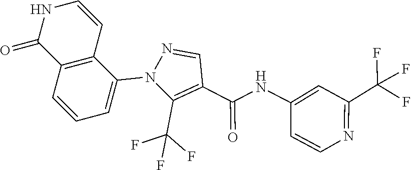 [SMILES: O=C(Nc1ccnc(C(F)(F)F)c1)c1cnn(-c2cccc3c(=O)[nH]ccc23)c1C(F)(F)F] |
| REO-1014981 (REO-981) | 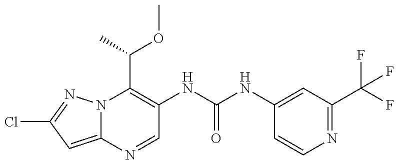 [SMILES: CO[C@@H](C)c1c(NC(=O)Nc2ccnc(C(F)(F)F)c2)cnc2cc(Cl)nn12] |
| REO-1017528 (REO-528) | 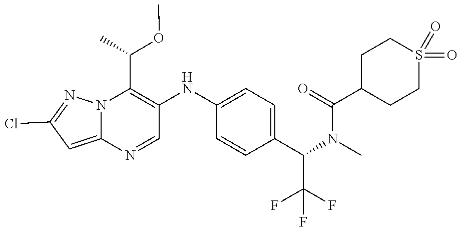 [SMILES: CO[C@@H](C)c1c(Nc2ccc([C@H](N(C)C(=O)C3CCS(=O)(=O)CC3)C(F)(F)F)cc2)cnc2cc(Cl)nn12] |
| REO-1017538 (REO-538) | 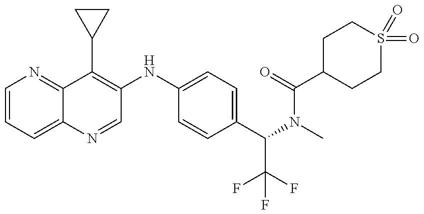 [SMILES: CN(C(=O)C1CCS(=O)(=O)CC1)[C@@H](c1ccc(Nc2cnc3cccnc3c2C2CC2)cc1)C(F)(F)F] |
| REO-1017703 (REO-703) | 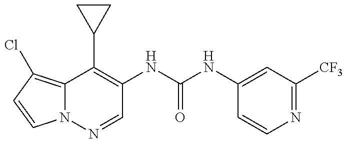 [SMILES: O=C(Nc1ccnc(C(F)(F)F)c1)Nc1cnn2ccc(Cl)c2c1C1CC1] |
| REO-1015076 (REO-076) | 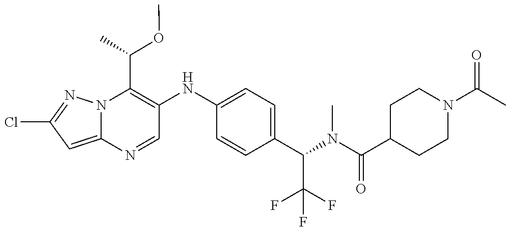 [SMILES: CO[C@@H](C)c1c(Nc2ccc([C@H](N(C)C(=O)C3CCN(C(C)=O)CC3)C(F)(F)F)cc2)cnc2cc(Cl)nn12] |

TABLE 1B
Additional Example MALT1 inhibitors

| Compound | Structure | Chemical Name |
|---|---|---|
| 1 | 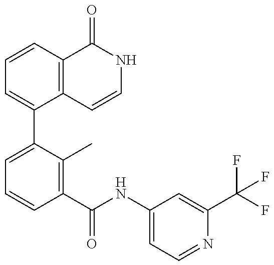 [SMILES: Cc1c(C(=O)Nc2ccnc(C(F)(F)F)c2)cccc1-c1cccc2c(=O)[nH]ccc12] | 2-methyl-3-(1-oxo-1,2- dihydroisoquinolin-5-yl)-N-(2- (trifluoromethyl)pyridin-4- yl)benzamide |
| 2 | 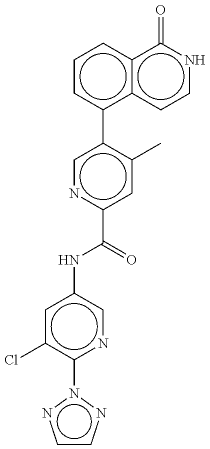 | N-(5-chloro-6-(2H-1,2,3-triazol- 2-yl)pyridin-3-yl)-4-methyl-5- (1-oxo-1,2-dihydroisoquinolin- 5-yl)picolinamide |
| 3 | 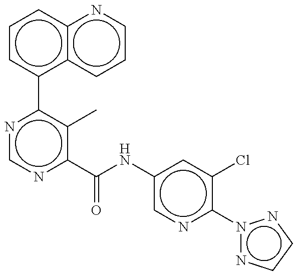 | N-(5-chloro-6-(2H-1,2,3-triazol- 2-yl)-4,5-dihydropyridin-3-yl)-6- (2,8-dihydroquinolin-5-yl)-5- methyl-5,6-dihydropyrimidine- 4-carboxamide |
| 4 | 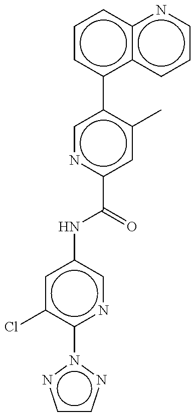 | N-(5-chloro-6-(2H-1,2,3-triazol- 2-yl)pyridin-3-yl)-4-methyl-5- (quinolin-5-yl)picolinamide |
| 5 | 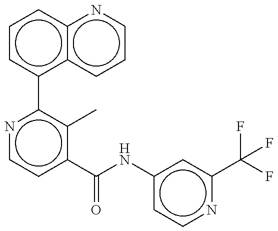 | 3-methyl-2-(quinolin-5-yl)-N-(2- (trifluoromethyl)pyridin-4- yl)isonicotinamide |
| 6 | 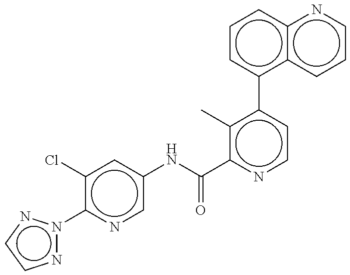 | N-(5-chloro-6-(2H-1,2,3-triazol- 2-yl)pyridin-3-yl)-3-methyl-4- (quinolin-5-yl)picolinamide |
| 7 | 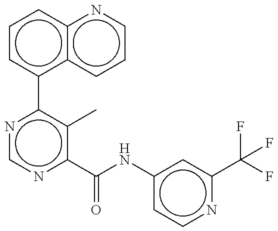 | 5-methyl-6-(quinolin-5-yl)-N-(2- (trifluoromethyl)pyridin-4- yl)pyrimidine-4-carboxamide |
| 8 | 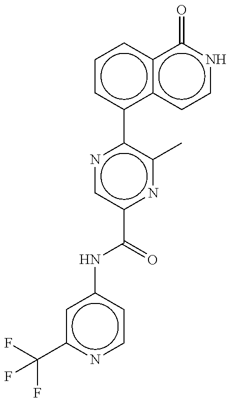 | 6-methyl-5-(1-oxo-1,2- dihydroisoquinolin-5-yl)-N-(2- (trifluoromethyl)pyridin-4- yl)pyrazine-2-carboxamide |
| 9 | 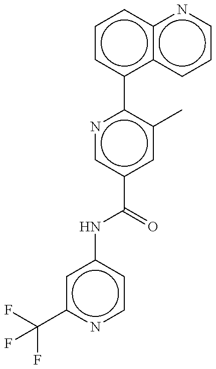 | 5-methyl-6-(quinolin-5-yl)-N-(2- (trifluoromethyl)pyridin-4- yl)nicotinamide |
| 10 | 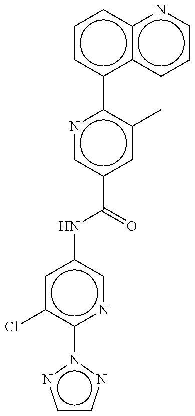 | N-(5-chloro-6-(2H-1,2,3-triazol- 2-yl)pyridin-3-yl)-5-methyl-6- (quinolin-5-yl)nicotinamide |
| 11 | 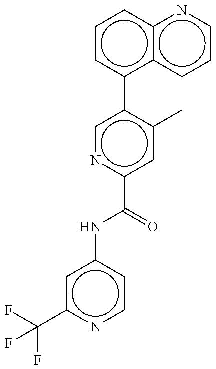 | 4-methyl-5-(quinolin-5-yl)-N-(2- (trifluoromethyl)pyridin-4- yl)picolinamide |
| 12 | 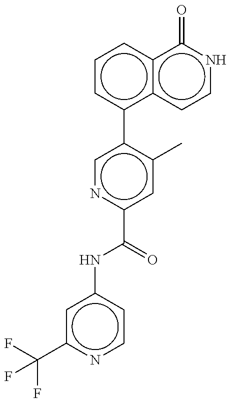 | 4-methyl-5-(1-oxo-1,2- dihydroisoquinolin-5-yl)-N-(2- (trifluoromethyl)pyridin-4- yl)picolinamide |
| 13 | 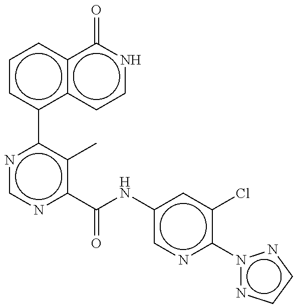 | N-(5-chloro-6-(2H-1,2,3-triazol- 2-yl)pyridin-3-yl)-5-methyl-6- (1-oxo-1,2-dihydroisoquinolin- 5-yl)pyrimidine-4-carboxamide |
| 14 | 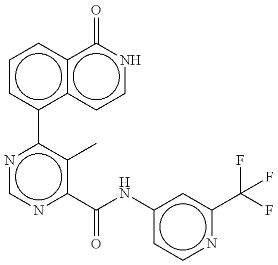 | 5-methyl-6-(1-oxo-1,2- dihydroisoquinolin-5-yl)-N-(2- (trifluoromethyl)pyridin-4- yl)pyrimidine-4-carboxamide |
| 15 | 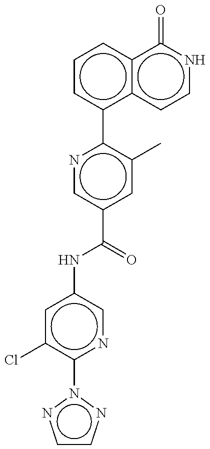 | N-(5-chloro-6-(2H-1,2,3-triazol- 2-yl)pyridin-3-yl)-5-methyl-6- (1-oxo-1,2-dihydroisoquinolin- 5-yl)nicotinamide |
| 16 | 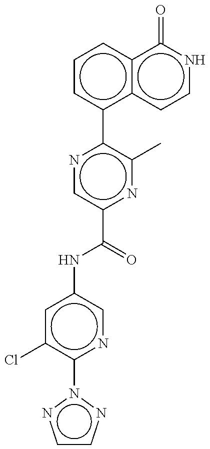 | N-(5-chloro-6-(1,3-dihydro-2H- 1,2,3-triazol-2-yl)pyridin-3-yl)- 6-methyl-5-(1-oxo-1,2- dihydroisoquinolin-5- yl)pyrazine-2-carboxamide |
| 17 | 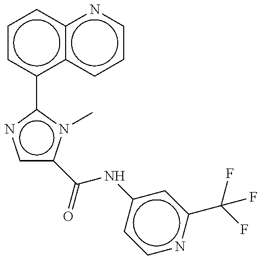 | 1-methyl-2-(quinolin-5-yl)-N-(2- (trifluoromethyl)pyridin-4-yl)- 1H-imidazole-5-carboxamide |
| 18 | 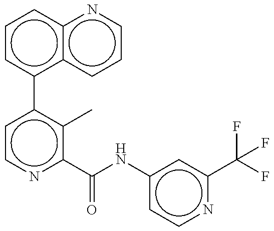 | 3-methyl-4-(quinolin-5-yl)-N-(2- (trifluoromethyl)pyridin-4- yl)picolinamide |
| 19 | 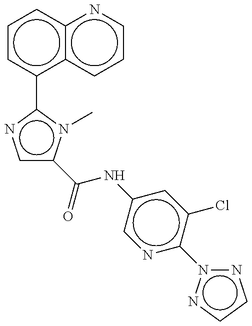 | N-(5-chloro-6-(112,214,3-triazol- 2-yl)pyridin-3-yl)-1-methyl-2- (quinolin-5-yl)-2,5-dihydro-1H- imidazole-5-carboxamide |
| 20 | 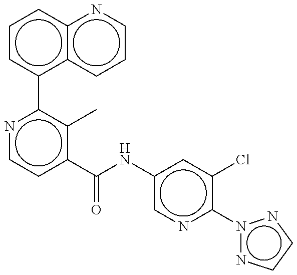 | N-(5-chloro-6-(2H-1,2,3-triazol- 2-yl)pyridin-3-yl)-3-methyl-2- (quinolin-5-yl)isonicotinamide |
| 21 | 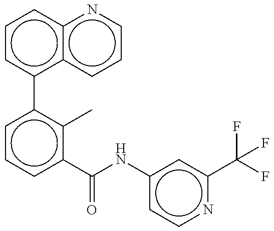 | 2-methyl-3-(quinolin-5-yl)-N-(2- (trifluoromethyl)-2,3- dihydropyridin-4-yl)benzamide |
| 22 | 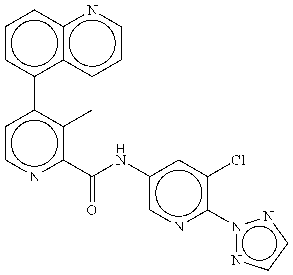 | N-(5-chloro-6-(2H-1,2,3-triazol- 2-yl)pyridin-3-yl)-2-methyl-3- (quinolin-5-yl)benzamide |
| 23 | 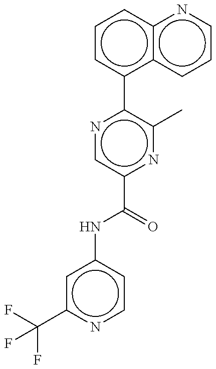 | 6-methyl-5-(quinolin-5-yl)-N-(2- (trifluoromethyl)pyridin-4- yl)pyrazine-2-carboxamide |
| 24 | 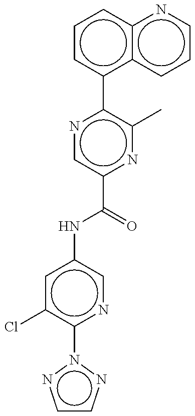 | N-(5-chloro-6-(1l2,2,3- triazolidin-2-yl)pyridin-3-yl)-6- methyl-5-(quinolin-5- yl)pyrazine-2-carboxamide |
| 25 | 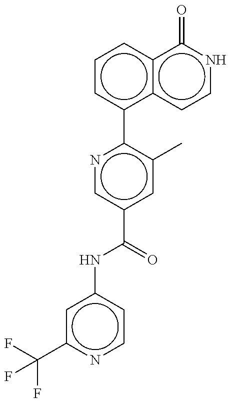 | 5-methyl-6-(1-oxo-1,2- dihydroisoquinolin-5-yl)-N-(2- (trifluoromethyl)pyridin-4- yl)nicotinamide |
| 26 | 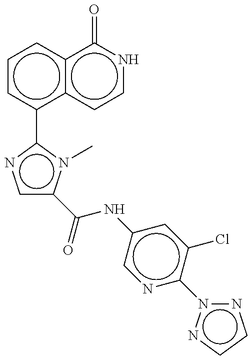 | N-(5-chloro-6-(1,3-dihydro-2H- 1,2,3-triazol-2-yl)-1,6- dihydropyridin-3-yl)-1-methyl- 2-(1-oxo-1,2- dihydroisoquinolin-5-yl)-4,5- dihydro-1H-imidazole-5- carboxamide |
| 27 | 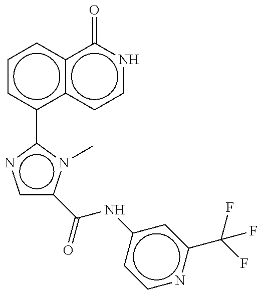 | 1-methyl-2-(1-oxo-1,2- dihydroisoquinolin-5-yl)-N-(2- (trifluoromethyl)pyridin-4-yl)- 1H-imidazole-5-carboxamide |
| 28 | 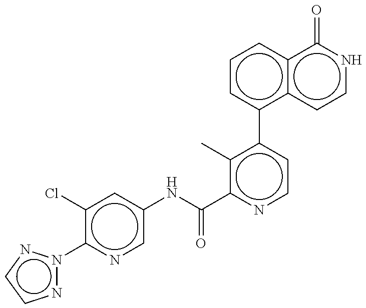 | N-(5-chloro-6-(2H-1,2,3-triazol- 2-yl)pyridin-3-yl)-3-methyl-4- (1-oxo-1,2-dihydroisoquinolin- 5-yl)picolinamide |
| 29 | 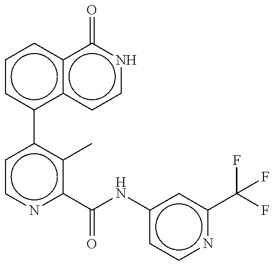 | 3-methyl-4-(1-oxo-1,2- dihydroisoquinolin-5-yl)-N-(2- (trifluoromethyl)pyridin-4- yl)picolinamide |
| 30 | 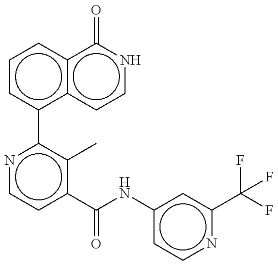 | 3-methyl-2-(1-oxo-1,2- dihydroisoquinolin-5-yl)-N-(2- (trifluoromethyl)pyridin-4- yl)isonicotinamide |
| 31 | 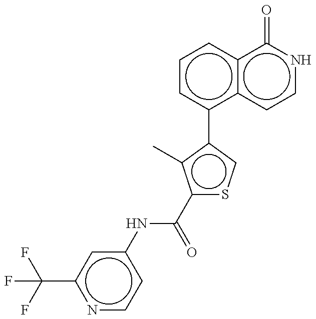 | 4-methyl-3-(1-oxo-1,2- dihydroisoquinolin-5-yl)-N-(2- (trifluoromethyl)pyridin-4-yl)- 2H-1l4-thiophene-5- carboxamide |
| 32 | 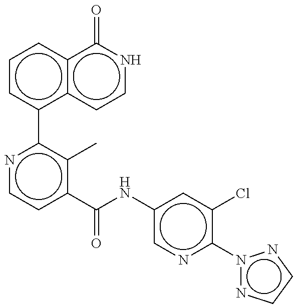 | N-(5-chloro-6-(2H-1,2,3-triazol- 2-yl)pyridin-3-yl)-3-methyl-2- (1-oxo-1,2-dihydroisoquinolin- 5-yl)isonicotinamide |
| 33 | 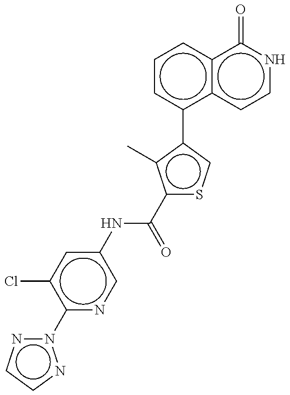 | N-(5-chloro-6-(2H-1,2,3-triazol- 2-yl)pyridin-3-yl)-3-methyl-4- (1-oxo-1,2-dihydroisoquinolin- 5-yl)thiophene-2-carboxamide |
| 34 | 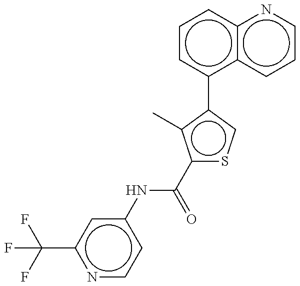 | 3-methyl-4-(quinolin-5-yl)-N-(6- (trifluoromethyl)-3,6-dihydro- 2H-1l2-pyridin-4-yl)thiophene- 2-carboxamide |
| 35 | 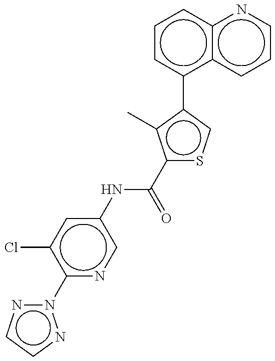 | 4-(2H-1l2-quinolin-5-yl)-N-(5- chloro-6-(1,2,3-triazolidin-2- yl)piperidin-3-yl)-3- methylthiophene-2-carboxamide |
| 36 | 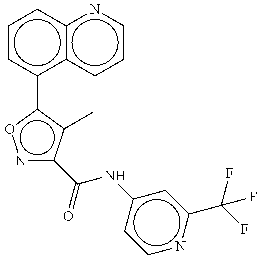 | 4-methyl-5-(quinolin-5-yl)-N-(2- (trifluoromethyl)pyridin-4- yl)isoxazole-3-carboxamide |
| 37 | 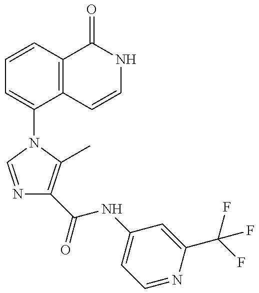 [SMILES: Cc1c(C(=O)Nc2ccnc(C(F)(F)F)c2)ncn1-c1cccc2c(=O)[nH]ccc12] | 5-methyl-1-(1-oxo-1,2- dihydroisoquinolin-5-yl)-N-(2- (trifluoromethyl)pyridin-4-yl)- 1H-imidazole-4-carboxamide |
| 38 | 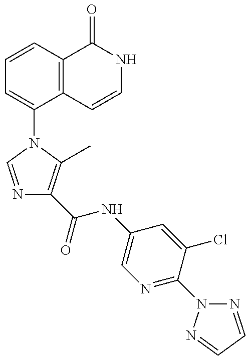 [SMILES: Cc1c(C(=O)Nc2cnc(-n3nccn3)c(Cl)c2)ncn1-c1cccc2c(=O)[nH]ccc12] | N-(5-chloro-6-(2H-1,2,3-triazol- 2-yl)pyridin-3-yl)-5-methyl-1- (1-oxo-1,2-dihydroisoquinolin- 5-yl)-1H-imidazole-4- carboxamide |
| 39 | 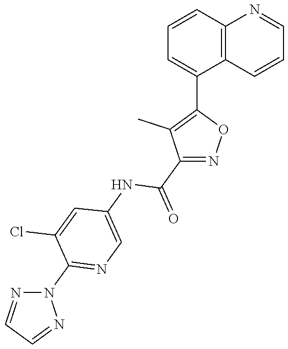 [SMILES: Cc1c(C(=O)Nc2cnc(-n3nccn3)c(Cl)c2)noc1-c1cccc2ncccc12] | N-(5-chloro-6-(2H-1,2,3-triazol- 2-yl)pyridin-3-yl)-4-methyl-5- (quinolin-5-yl)isoxazole-3- carboxamide |
| 40 | 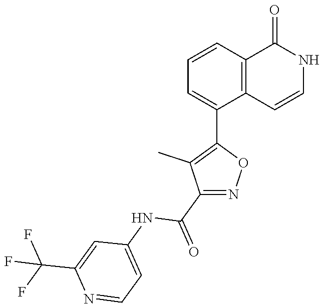 [SMILES: Cc1c(C(=O)Nc2ccnc(C(F)(F)F)c2)noc1-c1cccc2c(=O)[nH]ccc12] | 4-methyl-5-(1-oxo-1,2- dihydroisoquinolin-5-yl)-N-(2- (trifluoromethyl)pyridin-4- yl)isoxazole-3-carboxamide |
| 41 | 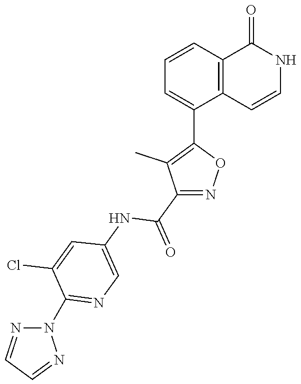 [SMILES: Cc1c(C(=O)Nc2cnc(-n3nccn3)c(Cl)c2)noc1-c1cccc2c(=O)[nH]ccc12] | N-(5-chloro-6-(2H-1,2,3-triazol- 2-yl)pyridin-3-yl)-4-methyl-5- (1-oxo-1,2-dihydroisoquinolin- 5-yl)isoxazole-3-carboxamide |
| 42 | 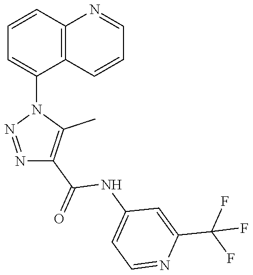 [SMILES: Cc1c(C(=O)Nc2ccnc(C(F)(F)F)c2)nnn1-c1cccc2ncccc12] | 5-methyl-1-(quinolin-5-yl)-N-(2- (trifluoromethyl)pyridin-4-yl)- 1H-1,2,3-triazole-4-carboxamide |
| 43 | 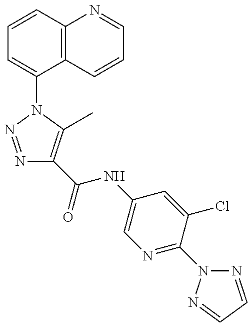 [SMILES: Cc1c(C(=O)Nc2cnc(-n3nccn3)c(Cl)c2)nnn1-c1cccc2ncccc12] | N-(5-chloro-6-(2H-1,2,3-triazol- 2-yl)pyridin-3-yl)-5-methyl-1- (quinolin-5-yl)-1H-1,2,3- triazole-4-carboxamide |
| 44 | 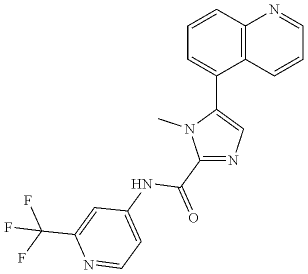 [SMILES: Cn1c(-c2cccc3ncccc23)cnc1C(=O)Nc1ccnc(C(F)(F)F)c1] | 1-methyl-5-(quinolin-5-yl)-N-(2- (trifluoromethyl)pyridin-4-yl)- 1H-imidazole-2-carboxamide |
| 45 | 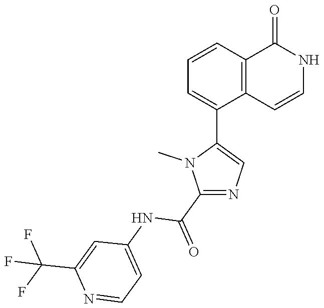 [SMILES: Cn1c(-c2cccc3c(=O)[nH]ccc23)cnc1C(=O)Nc1ccnc(C(F)(F)F)c1] | 1-methyl-5-(1-oxo-1,2- dihydroisoquinolin-5-yl)-N-(2- (trifluoromethyl)pyridin-4-yl)- 1H-imidazole-2-carboxamide |
| 46 | 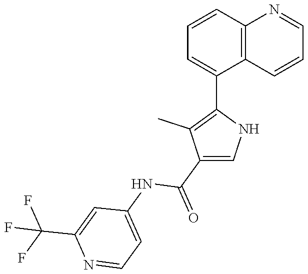 [SMILES: Cc1c(C(=O)Nc2ccnc(C(F)(F)F)c2)c[nH]c1-c1cccc2ncccc12] | 4-methyl-5-(quinolin-5-yl)-N-(2- (trifluoromethyl)pyridin-4-yl)- 1H-pyrazole-3-carboxamide |
| 47 | 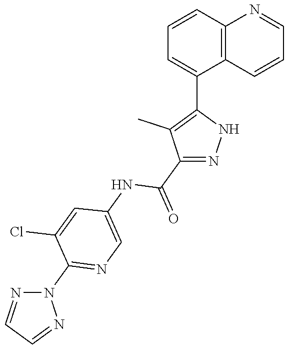 [SMILES: Cc1c(C(=O)Nc2cnc(-n3nccn3)c(Cl)c2)n[nH]c1-c1cccc2ncccc12] | N-(5-chloro-6-(2H-1,2,3-triazol- 2-yl)pyridin-3-yl)-4-methyl-5- (quinolin-5-yl)-1H-pyrazole-3- carboxamide |
| 48 | 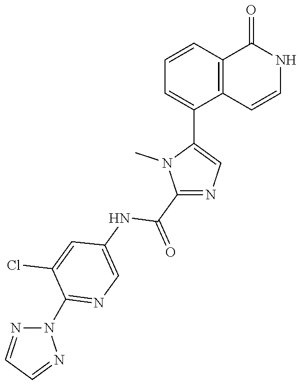 [SMILES: Cn1c(-c2cccc3c(=O)[nH]ccc23)cnc1C(=O)Nc1cnc(-n2nccn2)c(Cl)c1] | N-(5-chloro-6-(2H-1,2,3-triazol- 2-yl)pyridin-3-yl)-1-methyl-5- (1-oxo-1,2-dihydroisoquinolin- 5-yl)-1H-imidazole-2- carboxamide |
| 49 | 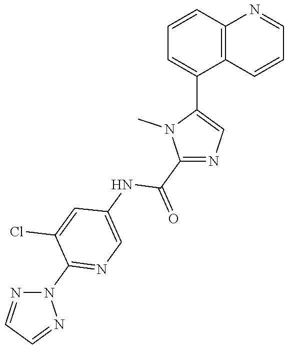 [SMILES: Cn1c(-c2cccc3ncccc23)cnc1C(=O)Nc1cnc(-n2nccn2)c(Cl)c1] | N-(5-chloro-6-(2H-1,2,3-triazol- 2-yl)pyridin-3-yl)-1-methyl-5- (quinolin-5-yl)-1H-imidazole-2- carboxamide |
| 50 | 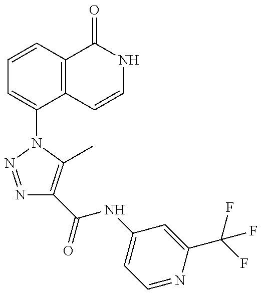 [SMILES: Cc1c(C(=O)Nc2ccnc(C(F)(F)F)c2)nnn1-c1cccc2c(=O)[nH]ccc12] | 5-methyl-1-(1-oxo-1,2- dihydroisoquinolin-5-yl)-N-(2- (trifluoromethyl)pyridin-4-yl)- 1H-1,2,3-triazole-4-carboxamide |
| 51 | 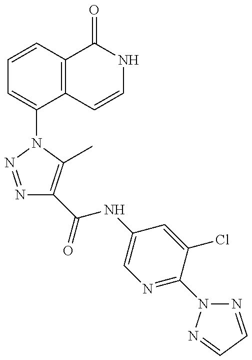 [SMILES: Cc1c(C(=O)Nc2cnc(-n3nccn3)c(Cl)c2)nnn1-c1cccc2c(=O)[nH]ccc12] | N-(5-chloro-6-(2H-1,2,3-triazol- 2-yl)pyridin-3-yl)-5-methyl-1- (1-oxo-1,2-dihydroisoquinolin- 5-yl)-1H-1,2,3-triazole-4- carboxamide |
| 52 | 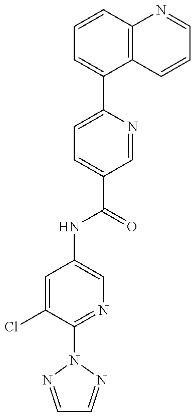 [SMILES: O=C(Nc1cnc(-n2nccn2)c(Cl)c1)c1ccc(-c2cccc3ncccc23)nc1] | N-(5-chloro-6-(2H-1,2,3-triazol- 2-yl)pyridin-3-yl)-6-(quinolin-5- yl)nicotinamide |
| 53 | 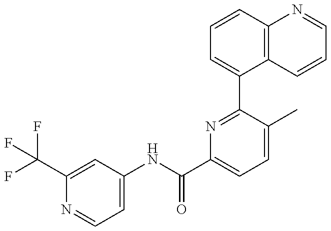 [SMILES: Cc1ccc(C(=O)Nc2ccnc(C(F)(F)F)c2)nc1-c1cccc2ncccc12] | 5-methyl-6-(quinolin-5-yl)-N-(2- (trifluoromethyl)pyridin-4- yl)picolinamide |
| 54 | 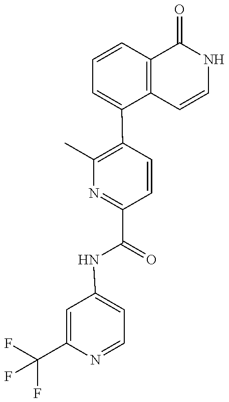 [SMILES: Cc1nc(C(=O)Nc2ccnc(C(F)(F)F)c2)ccc1-c1cccc2c(=O)[nH]ccc12] | 6-methyl-5-(1-oxo-1,2- dihydroisoquinolin-5-yl)-N-(2- (trifluoromethyl)pyridin-4- yl)picolinamide |
| 55 | 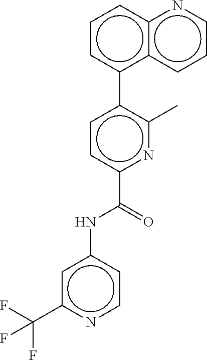 | 6-methyl-5-(quinolin-5-yl)-N-(2- (trifluoromethyl)pyridin-4-yl)- 5,6-dihydropyridine-2- carboxamide |
| 56 | 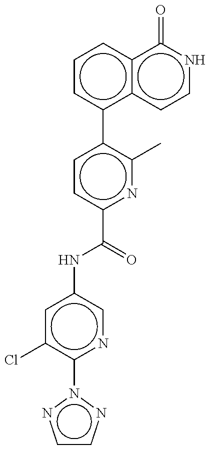 | N-(5-chloro-6-(2H-1,2,3-triazol- 2-yl)pyridin-3-yl)-6-methyl-5- (1-oxo-1,2-dihydroisoquinolin- 5-yl)picolinamide |
| 57 | 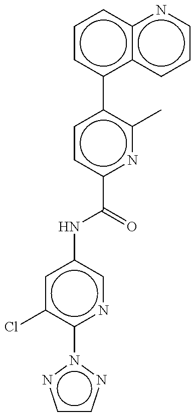 | N-(5-chloro-6-(2H-1,2,3-triazol- 2-yl)pyridin-3-yl)-6-methyl-5- (quinolin-5-yl)picolinamide |
| 58 | 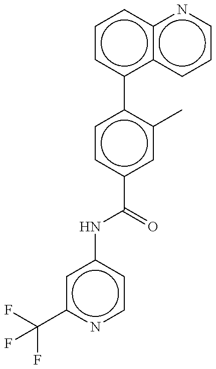 | 3-methyl-4-(quinolin-5-yl)-N-(2- (trifluoromethyl)pyridin-4- yl)benzamide |
| 59 | 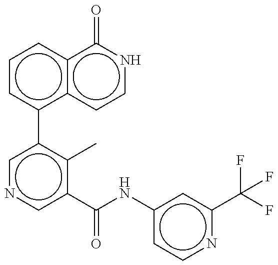 | 4-methyl-5-(1-oxo-1,2- dihydroisoquinolin-5-yl)-N-(2- (trifluoromethyl)pyridin-4- yl)nicotinamide |
| 60 | 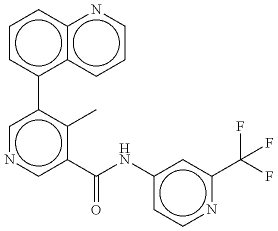 | 4-methyl-5-(quinolin-5-yl)-N-(2- (trifluoromethyl)pyridin-4- yl)nicotinamide |
| 61 | 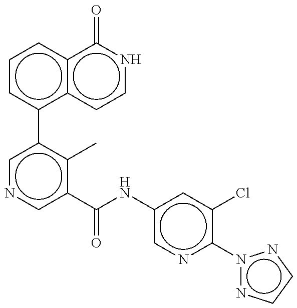 | N-(5-chloro-6-(2H-1,2,3-triazol- 2-yl)pyridin-3-yl)-4-methyl-5- (1-oxo-1,2-dihydroisoquinolin- 5-yl)nicotinamide |
| 62 | 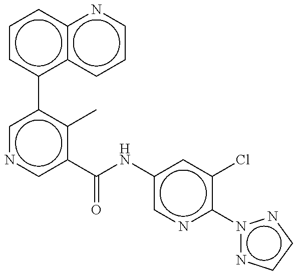 | N-(5-chloro-6-(1,3-dihydro-2H- 1,2,3-triazol-2-yl)pyridin-3-yl)- 4-methyl-5-(quinolin-5- yl)nicotinamide |
| 63 | 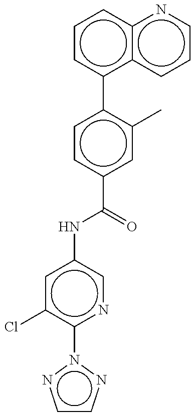 | N-(5-chloro-6-(2H-1,2,3-triazol- 2-yl)pyridin-3-yl)-3-methyl-4- (quinolin-5-yl)benzamide |
| 64 | 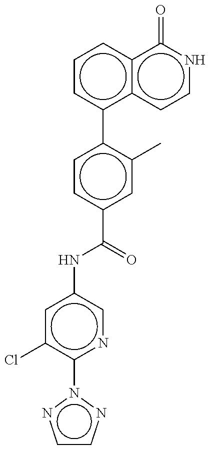 | N-(5-chloro-6-(2H-1,2,3-triazol- 2-yl)pyridin-3-yl)-3-methyl-4- (1-oxo-1,2-dihydroisoquinolin- 5-yl)benzamide |
| 65 | 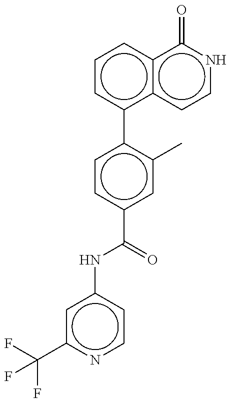 | 3-methyl-4-(1-oxo-1,2- dihydroisoquinolin-5-yl)-N-(2- (trifluoromethyl)pyridin-4- yl)benzamide |
| 66 | 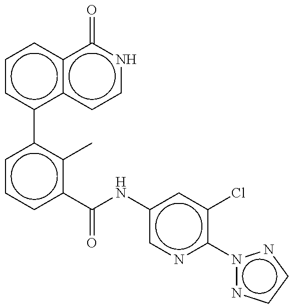 | N-(5-chloro-6-(2H-1,2,3-triazol- 2-yl)pyridin-3-yl)-2-methyl-3- (1-oxo-1,2-dihydroisoquinolin- 5-yl)benzamide |
| 67 | 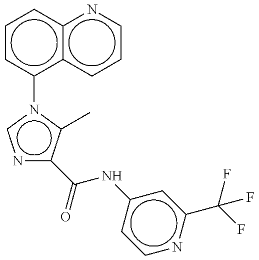 | 5-methyl-1-(quinolin-5-yl)-N-(2- (trifluoromethyl)pyridin-4-yl)- 1H-imidazole-4-carboxamide |
| 68 | 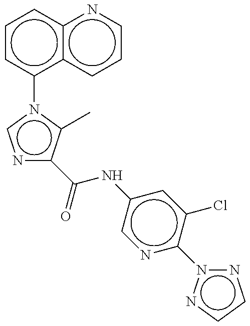 | N-(5-chloro-6-(2H-1,2,3-triazol- 2-yl)pyridin-3-yl)-5-methyl-1- (quinolin-5-yl)-1H-imidazole-4- carboxamide |
| 69 | 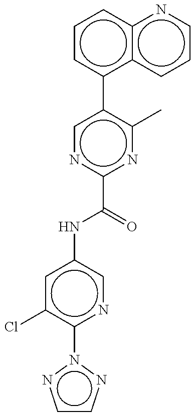 | N-(5-chloro-6-(2H-1,2,3-triazol- 2-yl)pyridin-3-yl)-4-methyl-5- (quinolin-5-yl)pyrimidine-2- carboxamide |
| 70 | 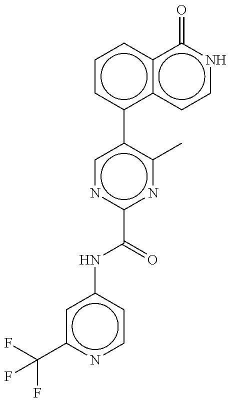 | 4-methyl-5-(1-oxo-1,2- dihydroisoquinolin-5-yl)-N-(2- (trifluoromethyl)pyridin-4- yl)pyrimidine-2-carboxamide |
| 71 | 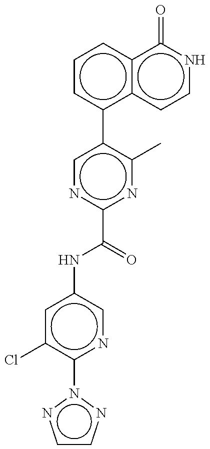 | N-(5-chloro-6-(2H-1,2,3-triazol- 2-yl)pyridin-3-yl)-4-methyl-5- (1-oxo-1,2-dihydroisoquinolin- 5-yl)pyrimidine-2-carboxamide |
| 72 | 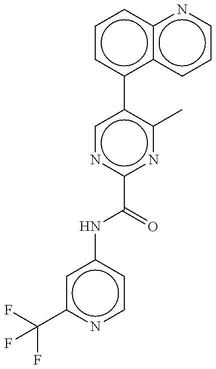 | 4-methyl-5-(quinolin-5-yl)-N-(2- (trifluoromethyl)pyridin-4-yl)- 4,5-dihydropyrimidine-2- carboxamide |
| 73 | 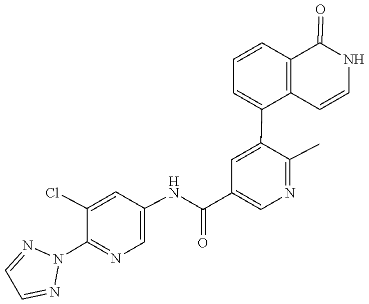 [SMILES: Cc1ncc(C(=O)Nc2cnc(-n3nccn3)c(Cl)c2)cc1-c1cccc2c(=O)[nH]ccc12] | N-(5-chloro-6-(2H-1,2,3-triazol- 2-yl)pyridin-3-yl)-6-methyl-5- (1-oxo-1,2-dihydroisoquinolin- 5-yl)nicotinamide |
| 74 | 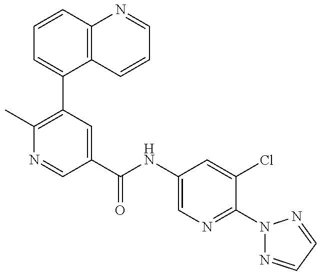 [SMILES: Cc1ncc(C(=O)Nc2cnc(-n3nccn3)c(Cl)c2)cc1-c1cccc2ncccc12] | N-(5-chloro-6-(2H-1,2,3-triazol- 2-yl)pyridin-3-yl)-6-methyl-5- (quinolin-5-yl)nicotinamide |
| 75 | 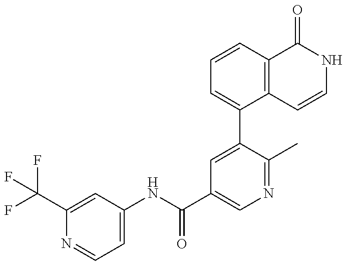 [SMILES: Cc1ncc(C(=O)Nc2ccnc(C(F)(F)F)c2)cc1-c1cccc2c(=O)[nH]ccc12] | 6-methyl-5-(1-oxo-1,2- dihydroisoquinolin-5-yl)-N-(2- (trifluoromethyl)pyridin-4- yl)nicotinamide |
| 76 | 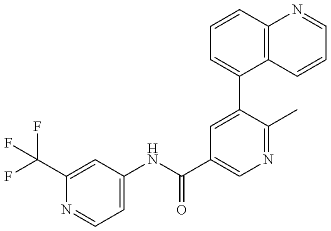 [SMILES: Cc1ncc(C(=O)Nc2ccnc(C(F)(F)F)c2)cc1-c1cccc2ncccc12] | 6-methyl-5-(quinolin-5-yl)-N-(2- (trifluoromethyl)pyridin-4- yl)nicotinamide |
| 77 | 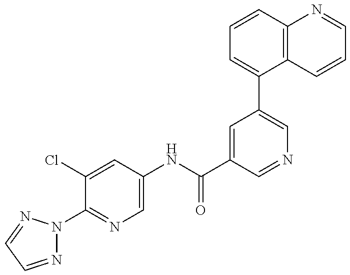 [SMILES: O=C(Nc1cnc(-n2nccn2)c(Cl)c1)c1cncc(-c2cccc3ncccc23)c1] | N-(5-chloro-6-(2H-1,2,3-triazol- 2-yl)pyridin-3-yl)-5-(quinolin-5- yl)nicotinamide |
| 78 | 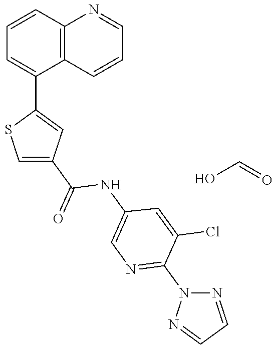 [SMILES: O=C(Nc1cnc(-n2nccn2)c(Cl)c1)c1csc(-c2cccc3ncccc23)c1 ; O=CO] | N-(5-chloro-6-(2H-1,2,3-triazol- 2-yl)pyridin-3-yl)-5-(quinolin-5- yl)thiophene-3-carboxamide formate |
| 79 | 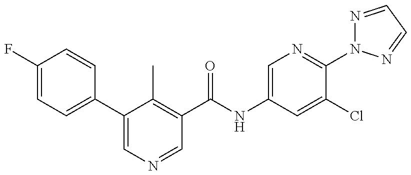 [SMILES: Cc1c(C(=O)Nc2cnc(-n3nccn3)c(Cl)c2)cncc1-c1ccc(F)cc1] | N-(5-chloro-6-(2H-1,2,3-triazol- 2-yl)pyridin-3-yl)-5-(4- fluorophenyl)-4- methylnicotinamide |
| 80 | 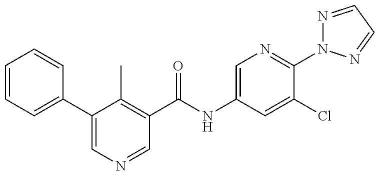 [SMILES: Cc1c(C(=O)Nc2cnc(-n3nccn3)c(Cl)c2)cncc1-c1ccccc1] | N-(5-chloro-6-(2H-1,2,3-triazol- 2-yl)pyridin-3-yl)-4-methyl-5- phenylnicotinamide |
| 81 | 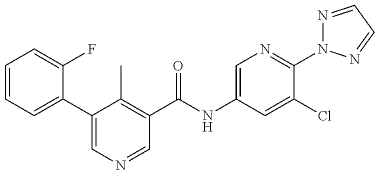 [SMILES: Cc1c(C(=O)Nc2cnc(-n3nccn3)c(Cl)c2)cncc1-c1ccccc1F] | N-(5-chloro-6-(2H-1,2,3-triazol- 2-yl)pyridin-3-yl)-5-(2- fluorophenyl)-4- methylnicotinamide |
| 82 | 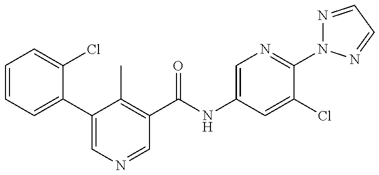 [SMILES: Cc1c(C(=O)Nc2cnc(-n3nccn3)c(Cl)c2)cncc1-c1ccccc1Cl] | N-(5-chloro-6-(2H-1,2,3-triazol- 2-yl)pyridin-3-yl)-5-(2- chlorophenyl)-4- methylnicotinamide |
| 83 | 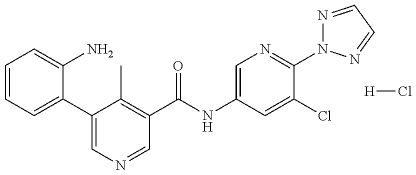 | 5-(2-aminophenyl)-N-(5-chloro- 6-(2H-1,2,3-triazol-2-yl)pyridin- 3-yl)-4-methylnicotinamide hydrochloride |
| 84 | 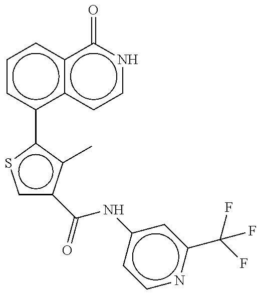 | 4-methyl-5-(1-oxo-1,2- dihydroisoquinolin-5-yl)-N-(2- (trifluoromethyl)pyridin-4- yl)thiophene-3-carboxamide |
| 85 | 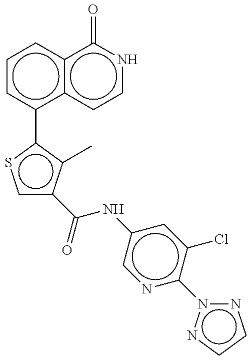 | N-(5-chloro-6-(2H-1,2,3-triazol- 2-yl)pyridin-3-yl)-4-methyl-5- (1-oxo-1,2-dihydroisoquinolin- 5-yl)thiophene-3-carboxamide |
| 86 | 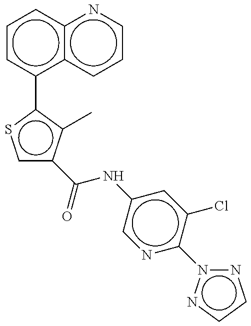 | N-(5-chloro-6-(2H-1,2,3-triazol- 2-yl)pyridin-3-yl)-4-methyl-5- (quinolin-5-yl)thiophene-3- carboxamide |
| 87 | 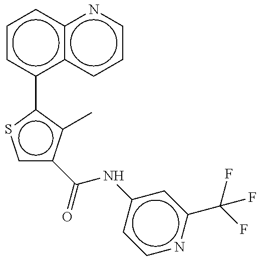 | 4-methyl-5-(quinolin-5-yl)-N-(2- (trifluoromethyl)pyridin-4- yl)thiophene-3-carboxamide |
| 88 | 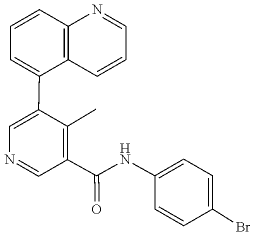 [SMILES: Cc1c(C(=O)Nc2ccc(Br)cc2)cncc1-c1cccc2ncccc12] | N-(4-bromophenyl)-4-methyl-5- (quinolin-5-yl)nicotinamide |
| 89 | 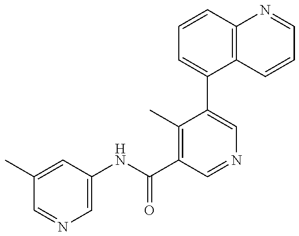 [SMILES: Cc1cncc(NC(=O)c2cncc(-c3cccc4ncccc34)c2C)c1] | 4-methyl-N-(5-methylpyridin-3- yl)-5-(quinolin-5- yl)nicotinamide |
| 90 | 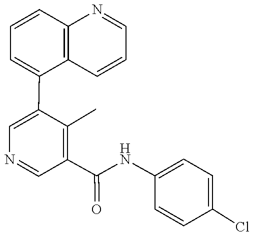 [SMILES: Cc1c(C(=O)Nc2ccc(Cl)cc2)cncc1-c1cccc2ncccc12] | N-(4-chlorophenyl)-4-methyl-5- (quinolin-5-yl)nicotinamide |
| 91 | 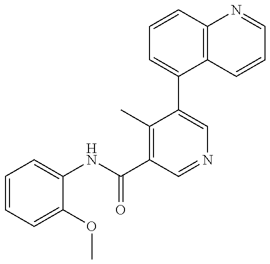 [SMILES: COc1ccccc1NC(=O)c1cncc(-c2cccc3ncccc23)c1C] | N-(2-methoxyphenyl)-4-methyl- 5-(quinolin-5-yl)nicotinamide |
| 92 | 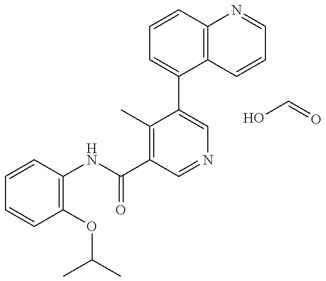 [SMILES: Cc1c(C(=O)Nc2ccccc2OC(C)C)cncc1-c1cccc2ncccc12 ; O=CO] | N-(2-isopropoxyphenyl)-4- methyl-5-(quinolin-5- yl)nicotinamide formate |
| 93 | 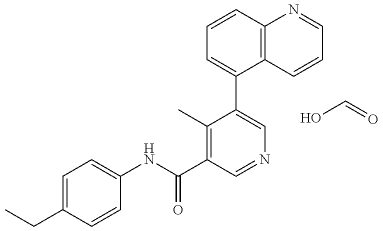 [SMILES: CCc1ccc(NC(=O)c2cncc(-c3cccc4ncccc34)c2C)cc1 ; O=CO] | N-(4-ethylphenyl)-4-methyl-5- (quinolin-5-yl)nicotinamide formate |
| 94 | 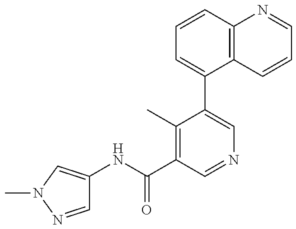 [SMILES: Cc1c(C(=O)Nc2cnn(C)c2)cncc1-c1cccc2ncccc12] | 4-methyl-N-(1-methyl-1H- pyrazol-4-yl)-5-(quinolin-5- yl)nicotinamide |
| 95 | 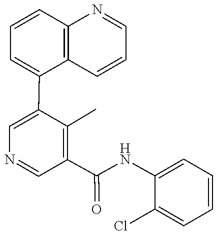 [SMILES: Cc1c(C(=O)Nc2ccccc2Cl)cncc1-c1cccc2ncccc12] | N-(2-chlorophenyl)-4-methyl-5- (quinolin-5-yl)nicotinamide |
| 96 | 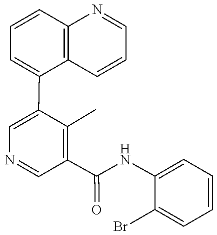 [SMILES: Cc1c(C(=O)Nc2ccccc2Br)cncc1-c1cccc2ncccc12] | N-(2-bromophenyl)-4-methyl-5- (quinolin-5-yl)nicotinamide |
| 97 | 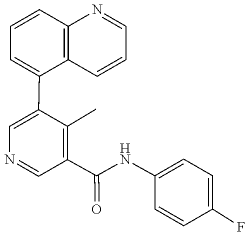 [SMILES: Cc1c(C(=O)Nc2ccc(F)cc2)cncc1-c1cccc2ncccc12] | N-(4-fluorophenyl)-4-methyl-5- (quinolin-5-yl)nicotinamide |
| 98 | 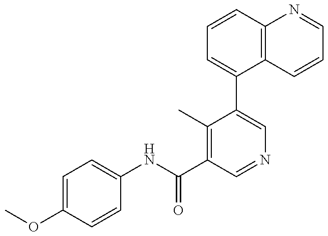 [SMILES: COc1ccc(NC(=O)c2cncc(-c3cccc4ncccc34)c2C)cc1] | N-(4-methoxyphenyl)-4-methyl- 5-(quinolin-5-yl)nicotinamide |
| 99 | 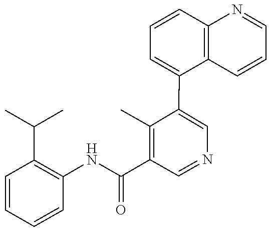 [SMILES: Cc1c(C(=O)Nc2ccccc2C(C)C)cncc1-c1cccc2ncccc12] | N-(2-isopropylphenyl)-4-methyl- 5-(quinolin-5-yl)nicotinamide |
| 100 | 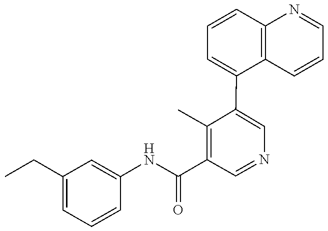 [SMILES: CCc1cccc(NC(=O)c2cncc(-c3cccc4ncccc34)c2C)c1] | N-(3-ethylphenyl)-4-methyl-5- (quinolin-5-yl)nicotinamide |
| 101 | 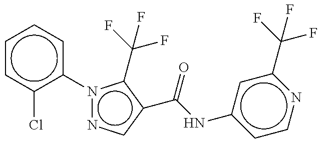 | 1-(2-chlorophenyl)-5- (trifluoromethyl)-N-(2- (trifluoromethyl)pyridin-4-yl)- 1H-pyrazole-4-carboxamide |
| 102 | 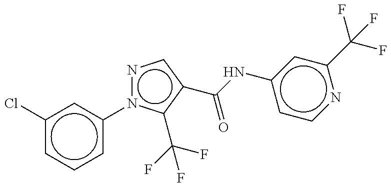 | 1-(3-chlorophenyl)-5- (trifluoromethyl)-N-(2- (trifluoromethyl)pyridin-4-yl)- 1H-pyrazole-4-carboxamide |
| 103 | 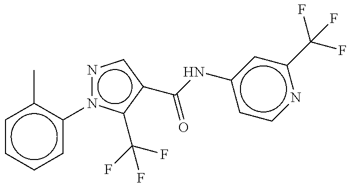 | 1-(o-tolyl)-5-(trifluoromethyl)- N-(2-(trifluoromethyl)pyridin-4- yl)-1H-pyrazole-4-carboxamide |
| 104 | 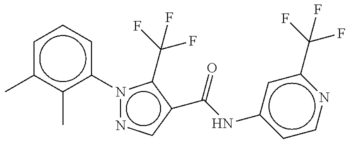 | 1-(2,3-dimethylphenyl)-5- (trifluoromethyl)-N-(2- (trifluoromethyl)pyridin-4-yl)- 1H-pyrazole-4-carboxamide |
| 105 | 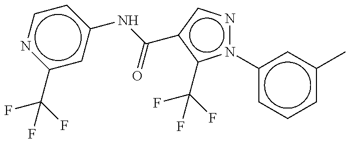 | 1-(m-tolyl)-5-(trifluoromethyl)- N-(2-(trifluoromethyl)pyridin-4- yl)-1H-pyrazole-4-carboxamide |
| 106 | 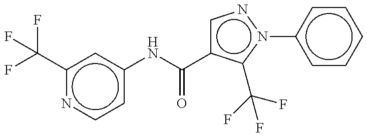 | 1-phenyl-5-(trifluoromethyl)-N- (2-(trifluoromethyl)pyridin-4- yl)-1H-pyrazole-4-carboxamide |
| 107 | 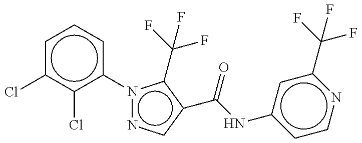 | 1-(2,3-dichlorophenyl)-5- (trifluoromethyl)-N-(2- (trifluoromethyl)pyridin-4-yl)- 1H-pyrazole-4-carboxamide |
| 108 | 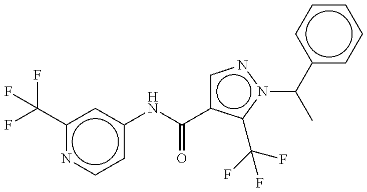 | 1-(1-phenylethyl)-5- (trifluoromethyl)-N-(2- (trifluoromethyl)pyridin-4-yl)- 1H-pyrazole-4-carboxamide |
| 109 | 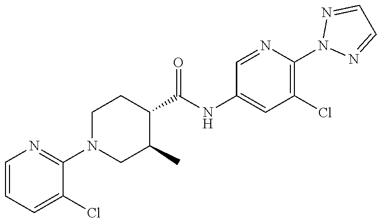 [SMILES: C[C@H]1CN(c2ncccc2Cl)CC[C@@H]1C(=O)Nc1cnc(-n2nccn2)c(Cl)c1] | (3R,4S)-N-(5-chloro-6-(2H- 1,2,3-triazol-2-yl)pyridin-3-yl)- 1-(3-chloropyridin-2-yl)-3- methylpiperidine-4-carboxamide |
| 110 | 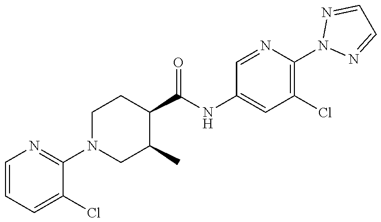 [SMILES: C[C@H]1CN(c2ncccc2Cl)CC[C@H]1C(=O)Nc1cnc(-n2nccn2)c(Cl)c1] | (3R,4R)-N-(5-chloro-6-(2H- 1,2,3-triazol-2-yl)pyridin-3-yl)- 1-(3-chloropyridin-2-yl)-3- methylpiperidine-4-carboxamide |
| 111 | 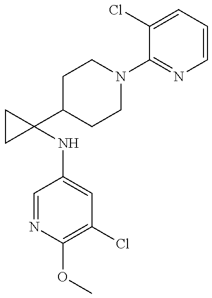 [SMILES: COc1ncc(NC2(C3CCN(c4ncccc4Cl)CC3)CC2)cc1Cl] | 5-chloro-N-(1-(1-(3- chloropyridin-2-yl)piperidin-4- yl)cyclopropyl)-6- methoxypyridin-3-amine |

### Pharmaceutical Compositions

Compounds provided in accordance with the present invention are usually administered in the form of pharmaceutical compositions. This invention therefore provides pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001, 139. Such patches may be constructed for continuous, pulsatile, or on demand delivery of pharmaceutical agents.

The compositions are preferably formulated in a unit dose form. The term “unit dose forms” refers to physically discrete units suitable as unitary doses for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dose forms such as tablets, pills and capsules.

The tablets or pills of the present invention may be coated or otherwise compounded to provide a dose form affording the advantage of prolonged action, or to protect from the acid conditions of the stomach. For example, the tablet or pill can comprise an inner dose and an outer dose component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprising a disclosed compound, or pharmaceutically acceptable salt thereof, and a pharmaceutically acceptable carrier.

### Example Diseases or Disorders

Compounds and compositions described herein are generally useful for modulating MALT1 and are useful for in treating diseases or disorders, in particular those susceptible to modulation of proteolytic and/or autoproteolytic activity of MALT1. In some embodiments, the compounds and compositions described herein are useful for inhibiting MALT1. In some embodiments, it is contemplated that the compounds and compositions of the present invention may be useful in the treatment of a disease, a disorder, or a condition characterized by dysregulated NF-KB activation, for example, autoimmune disorders, immunological disorders, inflammatory disorders, allergic disorders, respiratory disorders and oncological disorders. In some embodiments, it is contemplated that the compounds and compositions of the present invention may be useful in the treatment of a chronic disease or a chronic disorder, such as a chronic autoimmune disorder, chronic immunological disorder, or chronic inflammatory disorder.

In typical embodiments, the present invention is intended to encompass the compounds disclosed herein, and the pharmaceutically acceptable salts, pharmaceutically acceptable esters, tautomeric forms, polymorphs, and prodrugs of such compounds. In some embodiments, the present invention includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of a compound described herein.

In some embodiments, the autoimmune and inflammatory disorders are selected from arthritis, ankylosing spondylitis, inflammatory bowel disease, ulcerative colitis, gastritis, pancreatitis, Crohn's disease, celiac disease, primary sclerosing cholangitis, multiple sclerosis, Sjögren's syndrome, systemic sclerosis, scleritis, systemic lupus erythematosus, lupus nephritis, rheumatoid arthritis, rheumatic fever, gout, organ or transplant rejection, acute or chronic graft-versus-host disease, chronic allograft rejection, Behcet's disease, uveitis, psoriasis, psoriatic arthritis, BENTA disease, polymyositis, dermatitis, atopic dermatitis, dermatomyositis, acne vulgaris, myasthenia gravis, hidradenitis suppurativa, Grave's disease, Hashimoto thyroiditis, Sjogren's syndrome, and blistering disorders (e.g., pemphigus vulgaris), antibody-mediated vasculitis syndromes, including ANCA-associated vasculitis, Henoch-Schonlein Purpura, and immune-complex vasculitides (either primary or secondary to infection or cancers).

In some embodiments, the oncological disorders are selected from carcinoma, sarcoma, lymphoma, leukemia and germ cell tumors, adenocarcinoma, bladder cancer, clear cell carcinoma, skin cancer, brain cancer, cervical cancer, colon cancer, colorectal cancer, endometrial cancer, brain tumors, breast cancer, gastric cancer, germ cell tumors, glioblastoma, hepatic adenomas, Hodgkin's lymphoma, liver cancer, kidney cancer, lung cancer, pancreatic cancer, head/neck/throat cancer, ovarian cancer, dermal tumors, prostate cancer, renal cell carcinoma, stomach cancer, hematologic cancer, medulloblastoma, non-Hodgkin's lymphoma, diffuse large B-cell lymphoma (DLBCL), activated B cell-like diffuse large B Cell lymphoma (ABC-DLBCL), mantle cell lymphoma, marginal zone lymphoma, T cell lymphomas, in particular Sezary syndrome, Mycosis fungoides, cutaneous T-cell lymphoma, T-cell acute lymphoblastic leukemia, melanoma, mucosa-associated lymphoid tissue (MALT) lymphoma, multiple myeloma, plasma cell neoplasm, lentigo maligna melanomas, acral lentiginous melanoma, squamous cell carcinoma, chronic myelogenous leukemia, myeloid leukemia, superficial spreading melanoma, acral lentiginous melanoma, mucosal melanoma, nodular melanoma, polypoid melanoma, desmoplastic melanoma, amelanotic melanoma, soft-tissue melanoma, melanoma with small nevus-like cells, melanoma with features of a Spitz nevus, uveal melanoma, precursor T-cell, leukemia/lymphoma, acute myeloid leukemia, chronic myeloid leukemia, acute lymphocytic leukemia, follicular lymphoma, chronic lymphocytic leukemia/lymphoma, Burkitt's lymphoma, mycosis fungoides, peripheral T-cell lymphoma, nodular sclerosis form of Hodgkin lymphoma, mixed-cellularity subtype of Hodgkin lymphoma, non-small-cell lung cancer, large-cell carcinoma, and small-cell lung carcinoma.

In some embodiments, the oncological disorder is a cancer in the form of a tumor or a blood born cancer. In some embodiments, the tumor is a solid tumor. In some embodiments, the tumor is malignant and/or metastatic. In some embodiments, the tumor is selected from an adenoma, an adenocarcinoma, a blastoma (e.g., hepatoblastoma, glioblastoma, neuroblastoma and retinoblastoma), a carcinoma (e.g., colorectal carcinoma or hepatocellular carcinoma, pancreatic, prostate, gastric, esophageal, cervical, and head and neck carcinomas, and adenocarcinoma), a desmoid tumor, a desmoplastic small round cell tumor, an endocrine tumor, a germ cell tumor, a lymphoma, a leukemia, a sarcoma (e.g., Ewing sarcoma, osteosarcoma, rhabdomyosarcoma, or any other soft tissue sarcoma), a Wilms tumor, a lung tumor, a colon tumor, a lymph tumor, a breast tumor or a melanoma.

In some embodiments, the allergic disorder is selected from contact dermatitis, celiac disease, asthma, hypersensitivity to house dust mites, pollen and related allergens, and berylliosis.

In some embodiments, the respiratory disorder is selected from asthma, bronchitis, chronic obstructive pulmonary disease (COPD), cystic fibrosis, pulmonary edema, pulmonary embolism, pneumonia, pulmonary sarcoidosis, silicosis, pulmonary fibrosis, respiratory failure, acute respiratory distress syndrome, primary pulmonary hypertension and emphysema.

In some embodiments, the compounds and compositions of the present invention may be useful in the treatment of rheumatoid arthritis, systemic lupus erythematosus, vasculitic conditions, allergic diseases, asthma, chronic obstructive pulmonary disease (COPD), acute or chronic transplant rejection, graft versus host disease, cancers of hematopoietic origin or solid tumors, chronic myelogenous leukemia, myeloid leukemia, non-Hodgkin lymphoma or other B cell lymphomas.

In particular embodiments, the compounds and compositions of the present invention may be useful in the treatment of a chronic disease or a chronic disorder, such as a chronic autoimmune disorder, chronic immunological disorder, or chronic inflammatory disorder. Examples of chronic diseases or chronic disorders include any of chronic graft-versus-host disease (cGHVD), psoriatic arthritis, primary sclerosing cholangitis, multiple sclerosis, inflammatory bowel disease, Crohn's Disease, ulcerative colitis, psoriasis, Lupus, Sjögren's syndrome, scleritis, or rheumatoid arthritis.

In particular embodiments, the compounds and compositions of the present invention may be useful in the treatment of chronic graft-versus-host disease (cGHVD). In particular embodiments, the compounds and compositions of the present invention may be useful in the treatment of psoriatic arthritis. In particular embodiments, the compounds and compositions of the present invention may be useful in the treatment of primary sclerosing cholangitis. In particular embodiments, the compounds and compositions of the present invention may be useful in the treatment of multiple sclerosis. In particular embodiments, the compounds and compositions of the present invention may be useful in the treatment of inflammatory bowel disease. In particular embodiments, the compounds and compositions of the present invention may be useful in the treatment of Crohn's Disease. In particular embodiments, the compounds and compositions of the present invention may be useful in the treatment of ulcerative colitis. In particular embodiments, the compounds and compositions of the present invention may be useful in the treatment of psoriasis. In particular embodiments, the compounds and compositions of the present invention may be useful in the treatment of Lupus. In particular embodiments, the compounds and compositions of the present invention may be useful in the treatment of Sjögren's syndrome. In particular embodiments, the compounds and compositions of the present invention may be useful in the treatment of scleritis. In particular embodiments, the compounds and compositions of the present invention may be useful in the treatment of rheumatoid arthritis.

### Additional Embodiments

Disclosed herein is a method for treating a chronic disorder, the method comprising administering to the subject a MALT1 inhibitor, wherein following administration to the subject, the MALT1 inhibitor achieves a time over an IC50 blood concentration target from about 4 hours to about 20 hours per 24 hours. In various embodiments, following administration to the subject, the MALT1 inhibitor achieves a time over the blood concentration target from about 6 hours to about 18 hours per 24 hours. In various embodiments, following administration to the subject, the MALT1 inhibitor achieves a time over the blood concentration target from about 8 hours to about 16 hours per 24 hours. In various embodiments, following administration to the subject, the MALT1 inhibitor achieves a time over the blood concentration target from about 10 hours to about 14 hours per 24 hours. In various embodiments, the MALT1 inhibitor is administered at a dose from about 1 mg/kg to about 6 mg/kg. In various embodiments, the MALT1 inhibitor is administered at a dose from about 2 mg/kg to about 5 mg/kg. In various embodiments, the MALT1 inhibitor is administered at a dose of about 3 mg/kg.

In various embodiments, following administration to the subject, the MALT1 inhibitor achieves a time over a IC50 blood concentration target from about 12 hours to about 24 hours per 24 hours. In various embodiments, following the administration to the subject, the MALT1 inhibitor achieves a time over the IC50 blood concentration target from about 16 hours to about 24 hours per 24 hours. In various embodiments, following the administration to the subject, the MALT1 inhibitor achieves a time over the IC50 blood concentration target from about 22 hours to about 24 hours per 24 hours. In various embodiments, following the administration to the subject, the MALT1 inhibitor achieves a time over a IC90 blood concentration target from about 1 hour to about 15 hours per 24 hours. In various embodiments, following the administration to the subject, the MALT1 inhibitor achieves a time over a IC90 blood concentration target from about 3 hours to about 12 hours per 24 hours. In various embodiments, the administration to the subject, the MALT1 inhibitor achieves a time over a IC90 blood concentration target from about 6 hours to about 10 hours per 24 hours.

In various embodiments, the MALT1 inhibitor is administered at a dose from about 8 mg/kg to about 20 mg/kg. In various embodiments, the MALT1 inhibitor is administered at a dose from about 9 mg/kg to about 15 mg/kg. In various embodiments, the MALT1 inhibitor is administered at a dose of about 10 mg/kg. In various embodiments, the method comprising administering to the subject a MALT1 inhibitor, wherein following administration to the subject, the MALT1 inhibitor achieves a time over a IC90 blood concentration target from about 6 hours to about 24 hours per 24 hours. In various embodiments, following administration to the subject, the MALT1 inhibitor achieves a time over a IC90 blood concentration target from about 12 hours to about 24 hours per 24 hours. In various embodiments, following administration to the subject, the MALT1 inhibitor achieves a time over a IC90 blood concentration target from about 18 hours to about 24 hours per 24 hours. In various embodiments, following administration to the subject, the MALT1 inhibitor achieves a time over a IC90 blood concentration target from about 21 hours to about 24 hours per 24 hours.

In various embodiments, wherein the MALT1 inhibitor is administered at a dose from about 8 mg/kg to about 20 mg/kg. In various embodiments, the MALT1 inhibitor is administered at a dose from about 9 mg/kg to about 15 mg/kg. In various embodiments, the MALT1 inhibitor is administered at a dose of about 10 mg/kg.

Additionally disclosed herein is a method for treating a chronic disorder, the method comprising administering to the subject a MALT1 inhibitor, wherein following administration to the subject, the MALT1 inhibitor achieves a log₁₀(AUC) from about 0.5 μg*hr/mL to about 2.0 μg*hr/mL. In various embodiments, following administration to the subject, the MALT1 inhibitor achieves a log₁₀(AUC) from about 1.0 μg*hr/mL to about 1.75 μg*hr/mL. In various embodiments, following administration to the subject, the MALT1 inhibitor achieves a log₁₀(AUC) from about 1.25 μg*hr/mL to about 1.50 μg*hr/mL. In various embodiments, following administration of the MALT1 inhibitor to the subject, a level of Tregs of the subject remains at least 60% of a level prior to administration. In various embodiments, following administration of the MALT1 inhibitor to the subject, a level of Tregs of the subject remains at least 70% of a level prior to administration. In various embodiments, following administration of the MALT1 inhibitor to the subject, a level of Tregs of the subject remains at least 80% of a level prior to administration. In various embodiments, following administration of the MALT1 inhibitor to the subject, a level of Tregs of the subject remains at least 90% of a level prior to administration. In various embodiments, following administration of the MALT1 inhibitor to the subject, a level of Tregs of the subject remains at least 95% of a level prior to administration.

Additionally disclosed herein is a method for treating a chronic disorder, the method comprising administering to the subject a MALT1 inhibitor, wherein the MALT1 inhibitor is administered at a dose between from about 1 mg/kg to about 100 mg/kg. In various embodiments, the MALT1 inhibitor is administered at a dose between from about 20 mg/kg to about 40 mg/kg. In various embodiments, the MALT1 inhibitor is administered at a dose between from about 25 mg/kg to about 35 mg/kg. In various embodiments, the MALT1 inhibitor is administered at a dose of about 30 mg/kg. In various embodiments, the MALT1 inhibitor is administered at a dose between from about 5 mg/kg to about 15 mg/kg. In various embodiments, the MALT1 inhibitor is administered at a dose between from about 8 mg/kg to about 12 mg/kg. In various embodiments, the MALT1 inhibitor is administered at a dose of about 10 mg/kg. In various embodiments, the MALT1 inhibitor is administered at a dose between from about 2 mg/kg to about 10 mg/kg. In various embodiments, the MALT1 inhibitor is administered at a dose from about 2 mg/kg to about 5 mg/kg. In various embodiments, the MALT1 inhibitor is administered at a dose of about 3 mg/kg. In various embodiments, the MALT1 inhibitor is administered at a dose between from about 1 mg/kg to about 5 mg/kg. In various embodiments, the MALT1 inhibitor is administered at a dose from about 1 mg/kg to about 3 mg/kg. In various embodiments, the MALT1 inhibitor is administered at a dose of about 1 mg/kg. In various embodiments, the MALT1 inhibitor is administered intravenously. In various embodiments, the MALT1 inhibitor is locally administered.

In various embodiments, the MALT1 inhibitor is administered daily for between 5 to 20 days. In various embodiments, the MALT1 inhibitor is administered daily for between 5 to 8 days. In various embodiments, the MALT1 inhibitor is administered daily for 7 days. In various embodiments, the MALT1 inhibitor is administered daily for between 10 to 15 days. In various embodiments, the MALT1 inhibitor is administered daily for 14 days. In various embodiments, the MALT1 inhibitor is administered for one or more cycles, wherein a cycle comprises: administering the MALT1 inhibitor once per day for between 1-2 weeks followed by no treatment for 1-2 weeks. In various embodiments, a cycle comprises: administering the MALT1 inhibitor once per day for 2 weeks followed by no treatment for 1 week.

In various embodiments, wherein reduction of a level of Tregs of the subject with the chronic disorder following administration of the MALT1 inhibitor is lower in comparison to reduction of a level of Tregs of a healthy subject who receives the MALT1 inhibitor.

Disclosed herein is a method for treating a chronic disorder, the method comprising administering to the subject a MALT1 inhibitor, wherein following administration to the subject, the MALT1 inhibitor achieves a time over an IC50 blood concentration target from about 4 hours to about 20 hours per 24 hours. In various embodiments, following administration to the subject, the MALT1 inhibitor achieves a time over the blood concentration target from about 6 hours to about 18 hours per 24 hours. In various embodiments, following administration to the subject, the MALT1 inhibitor achieves a time over the blood concentration target from about 8 hours to about 16 hours per 24 hours. In various embodiments, following administration to the subject, the MALT1 inhibitor achieves a time over the blood concentration target from about 10 hours to about 14 hours per 24 hours. In various embodiments, the MALT1 inhibitor is administered at a dose from about 1 mg/kg to about 6 mg/kg. In various embodiments, the MALT1 inhibitor is administered at a dose from about 2 mg/kg to about 5 mg/kg. In various embodiments, the MALT1 inhibitor is administered at a dose of about 3 mg/kg.

In various embodiments, following administration to the subject, the MALT1 inhibitor achieves a time over a IC50 blood concentration target from about 12 hours to about 24 hours per 24 hours. In various embodiments, following the administration to the subject, the MALT1 inhibitor achieves a time over the IC50 blood concentration target from about 16 hours to about 24 hours per 24 hours. In various embodiments, following the administration to the subject, the MALT1 inhibitor achieves a time over the IC50 blood concentration target from about 22 hours to about 24 hours per 24 hours. In various embodiments, following the administration to the subject, the MALT1 inhibitor achieves a time over a IC90 blood concentration target from about 1 hour to about 15 hours per 24 hours. In various embodiments, following the administration to the subject, the MALT1 inhibitor achieves a time over a IC90 blood concentration target from about 3 hours to about 12 hours per 24 hours. In various embodiments, the administration to the subject, the MALT1 inhibitor achieves a time over a IC90 blood concentration target from about 6 hours to about 10 hours per 24 hours.

In various embodiments, the MALT1 inhibitor is administered at a dose from about 8 mg/kg to about 20 mg/kg. In various embodiments, the MALT1 inhibitor is administered at a dose from about 9 mg/kg to about 15 mg/kg. In various embodiments, the MALT1 inhibitor is administered at a dose of about 10 mg/kg. In various embodiments, the method comprising administering to the subject a MALT1 inhibitor, wherein following administration to the subject, the MALT1 inhibitor achieves a time over a IC90 blood concentration target from about 6 hours to about 24 hours per 24 hours. In various embodiments, following administration to the subject, the MALT1 inhibitor achieves a time over a IC90 blood concentration target from about 12 hours to about 24 hours per 24 hours. In various embodiments, following administration to the subject, the MALT1 inhibitor achieves a time over a IC90 blood concentration target from about 18 hours to about 24 hours per 24 hours. In various embodiments, following administration to the subject, the MALT1 inhibitor achieves a time over a IC90 blood concentration target from about 21 hours to about 24 hours per 24 hours.

In various embodiments, wherein the MALT1 inhibitor is administered at a dose from about 8 mg/kg to about 20 mg/kg. In various embodiments, the MALT1 inhibitor is administered at a dose from about 9 mg/kg to about 15 mg/kg. In various embodiments, the MALT1 inhibitor is administered at a dose of about 10 mg/kg.

Additionally disclosed herein is a method for treating a chronic disorder, the method comprising administering to the subject a MALT1 inhibitor, wherein following administration to the subject, the MALT1 inhibitor achieves a log₁₀(AUC) from about 0.5 μg*hr/mL to about 2.0 μg*hr/mL. In various embodiments, following administration to the subject, the MALT1 inhibitor achieves a log₁₀(AUC) from about 1.0 μg*hr/mL to about 1.75 μg*hr/mL. In various embodiments, following administration to the subject, the MALT1 inhibitor achieves a log₁₀(AUC) from about 1.25 μg*hr/mL to about 1.50 μg*hr/mL. In various embodiments, following administration of the MALT1 inhibitor to the subject, a level of Tregs of the subject remains at least 60% of a level prior to administration. In various embodiments, following administration of the MALT1 inhibitor to the subject, a level of Tregs of the subject remains at least 70% of a level prior to administration. In various embodiments, following administration of the MALT1 inhibitor to the subject, a level of Tregs of the subject remains at least 80% of a level prior to administration. In various embodiments, following administration of the MALT1 inhibitor to the subject, a level of Tregs of the subject remains at least 90% of a level prior to administration. In various embodiments, following administration of the MALT1 inhibitor to the subject, a level of Tregs of the subject remains at least 95% of a level prior to administration.

Additionally disclosed herein is a method for treating a chronic disorder, the method comprising administering to the subject a MALT1 inhibitor, wherein the MALT1 inhibitor is administered at a dose between from about 1 mg/kg to about 100 mg/kg. In various embodiments, the MALT1 inhibitor is administered at a dose between from about 20 mg/kg to about 40 mg/kg. In various embodiments, the MALT1 inhibitor is administered at a dose between from about 25 mg/kg to about 35 mg/kg. In various embodiments, the MALT1 inhibitor is administered at a dose of about 30 mg/kg. In various embodiments, the MALT1 inhibitor is administered at a dose between from about 5 mg/kg to about 15 mg/kg. In various embodiments, the MALT1 inhibitor is administered at a dose between from about 8 mg/kg to about 12 mg/kg. In various embodiments, the MALT1 inhibitor is administered at a dose of about 10 mg/kg. In various embodiments, the MALT1 inhibitor is administered at a dose between from about 2 mg/kg to about 10 mg/kg. In various embodiments, the MALT1 inhibitor is administered at a dose from about 2 mg/kg to about 5 mg/kg. In various embodiments, the MALT1 inhibitor is administered at a dose of about 3 mg/kg. In various embodiments, the MALT1 inhibitor is administered at a dose between from about 1 mg/kg to about 5 mg/kg. In various embodiments, the MALT1 inhibitor is administered at a dose from about 1 mg/kg to about 3 mg/kg. In various embodiments, the MALT1 inhibitor is administered at a dose of about 1 mg/kg. In various embodiments, the MALT1 inhibitor is administered intravenously. In various embodiments, the MALT1 inhibitor is locally administered.

In various embodiments, the MALT1 inhibitor is administered daily for between 5 to 20 days. In various embodiments, the MALT1 inhibitor is administered daily for between 5 to 8 days. In various embodiments, the MALT1 inhibitor is administered daily for 7 days. In various embodiments, the MALT1 inhibitor is administered daily for between 10 to 15 days. In various embodiments, the MALT1 inhibitor is administered daily for 14 days. In various embodiments, the MALT1 inhibitor is administered for one or more cycles, wherein a cycle comprises: administering the MALT1 inhibitor once per day for between 1-2 weeks followed by no treatment for 1-2 weeks. In various embodiments, a cycle comprises: administering the MALT1 inhibitor once per day for 2 weeks followed by no treatment for 1 week.

In various embodiments, wherein reduction of a level of Tregs of the subject with the chronic disorder following administration of the MALT1 inhibitor is lower in comparison to reduction of a level of Tregs of a healthy subject who receives the MALT1 inhibitor.

### EXAMPLES

The following examples are put forth so as to provide those of ordinary skill in the art with a description of how the compositions and methods described herein may be used, made, and evaluated, and are intended to be purely exemplary and are not intended to limit the scope of the invention.

### Example 1: Materials and Methods Involving Use of MALT1 Inhibitors

The subsequent Examples (e.g., Examples 2-6) involve the use of MALT1 inhibitors. Example 1 herein describes materials and methods for performing the experiments of Examples 2-6.

### Compound Synthesis and Characterization

The primary compound used in this study (described herein as “REO-981” and designated in the Examples as “MALT1i”) was synthesized as per the published protocol described in Martin, K. et al., Pharmacological Inhibition of MALT1 Protease Leads to a Progressive IPEX-Like Pathology. Front Immunol (2020) 11:745, which is hereby incorporated by reference in its entirety. Biochemical characterization of the compound was performed via measurement of enzymatic activity against target. Relative and longitudinal effect of the compound across MALT1 scaffolding and paracaspase/protease functions was determined by immunoblotting of cell lysates prepared from activated primary human CD4⁺ T-cells treated with MALT1i.

### Whole Blood Potency Assay

Heparinized human whole blood was obtained from healthy donors (Research Blood Components, Watertown, MA). Blood samples were diluted 1:1 with RPMI 1640 (Thermofisher Scientific, Waltham, MA) and aliquoted into 96-well plates with a final volume of 100 μl. Samples were treated with different concentrations of MALT1i from 0-30 μM in a volume of 10 μl and incubated for 1 h at 37° C. following which stimulation of the treated blood was performed with 1 μg/ml each of α-CD3 (clone UCHT1, Thermofisher) and α-CD28 (clone CD28.2, Thermofisher) in a volume of 10 μl and incubated for 48 h at 37° C.+5% CO₂. Plates were spun 470×g for 10 min and ˜40 μl of supernatant (plasma) separated from each well and stored at −80° C. prior to cytokine analysis.

Heparinized whole blood was obtained from naïve 8-10-week-old Sprague Dawley rats (Charles River Laboratories, Wilmington, MA). Treatment of 1:1 diluted blood samples were performed similarly as with the human whole blood. Following treatment with MALT1i samples were incubated with different concentrations of MALT1i from 0-30 μM. Following the initial incubation, the blood was stimulated with 10 μl of phorbol 12-myristate 13-acetate (PMA)/Ionomycin (Sigma, St. Louis, MO) at concentrations of 25 ng/ml and 1 μg/ml, respectively and as previously described. Plates were then incubated for 6 h at 37° C.+5% CO₂ for 6 h, spun down and the supernatant separated as described for the human whole assay. Samples were stored at −80° C. prior to cytokine analysis.

### Jurkat IL-2 Assay

Jurkat human T-cell line (ATCC, clone E6.1) was exposed to a range MALT1i concentrations and assessed for viability and inhibition of cytokine expression following cell activation. Cells were cultured in RPMI/10% FBS (Thermofisher) and maintained under a concentration of 3×10⁶ cells/ml. MALT1i at different concentrations were stamped by ECHO onto 384-well plates (PerkinElmer, Waltham, MA) following which cells were plated in fresh media and incubated for 30 minutes before stimulation with soluble α-CD3/CD28/CD2 (ImmunoCult, Stemcell Technologies, Vancouver, Canada) for 24 h. Supernatants were collected as mentioned earlier and processed immediately for cytokine analysis or stored at −80° C. To assess viability of cells treated with compound, cells were lysed with CTG reagent (Promega, Madison, WI), and measured by luminometer.

### Primary Human Cell Assays

Total CD4⁺ T-cells were isolated from human donor PBMCs using an EasySep kit (Stemcell) and were pre-incubated with REO-981 (0-5 μM) for 30 min at 37° C., 5% CO2 prior to stimulation with ImmunoCult Human CD3/CD28 T-cell activator (Stemcell) per manufacturer's protocol.

Memory CD4⁺ T-cells were isolated from human donor PBMCs using an EasySep kit (Stemcell) and were rested overnight in complete RPMI media (Thermofisher) supplemented with 10% FBS (Atlanta Biologics), 10 mM HEPES, 2 mM GlutaMAX, 1 mM Na Pyruvate, and 1×MEM non-essential amino acids (Thermofisher). Cells were treated with varying ratios of MALT1i (0-30 μM) for 30 min at 37° C., 5% CO₂ prior to stimulation with ImmunoCult Human CD3/CD28/CD2 T-cell activator (Stemcell).

Human monocytes were isolated from human donors using an EasySep kit (Stemcell) and were differentiated into macrophages in RPMI-1640 medium, supplemented with 10% FBS, 2 mM GlutaMAX, 100 IU/ml penicillin and 100 IU/ml streptomycin (Thermofisher) for 6 days in petri dishes in the presence of 50 g/ml M-CSF (Peprotech, Cranbury, NJ). After differentiation, cells were harvested with Accutase solution (Stemcell Technologies) and incubated for 2 h at 37° C., 5% CO₂ in 96-well plates. Cells were treated with varying ratios of MALT1i (0-30 μM) for 30 min at 37° C. prior to stimulation with 50 μg/ml depleted-zymosan (Invivogen, San Diego, CA) or IgG Immune Complex. For IgG Immune Complex preparation, 92 μg/ml of Anti-Human IgG (Jackson Immunoresearch, West Grove, PA) was gently laid on top of 10 μg/ml of Human IgG (Bio-Rad, Hercules, CA) and was incubated for 1 h at 37° C., 5% CO₂ prior to stimulating the cells.

Freshly isolated total CD19⁺ B-cells (Stemcell) from Human PBMCs were labeled with 2 μM Cell Trace Violet (ThermoFisher) and incubated for 5 min on a Nutator. Cells were then washed, plated and treated with MALT1i (0-1 μM) for 30 min at 37° C., 5% CO₂ prior to stimulation for 4 days with 50 μg/ml soluble F(ab′)2 anti-human IgA+IgG+IgM (H+L) (Jackson Immunoresearch, West Grove, PA), 100 ng/ml soluble recombinant CD40L (TNFSF5) (ThermoFisher). Proliferation was assessed by flow cytometry.

### Rat Collagen-Induced Arthritis Model

Adult female Lewis rats (Charles River) with body weights between 180-200 g were immunized subcutaneously with bovine type II collagen (Chondrex, Woodinville, WA)/Incomplete Freund's Adjuvant (Sigma) emulsion prepared per manufacturer's protocol (Chondrex) on Day 0 and Day 7. MALT1i doses for oral administration were prepared by suspending the compound in 0.5% Na-carboxymethylcellulose/0.5% Tween-80 in water (vehicle). For prophylactic treatment animals were dosed via oral gavage on Day 0 prior to immunization with collagen and continued once daily (q.d.) for four weeks. For therapeutic treatment, animals were randomized per clinical disease scoring on Day 14 and q.d. dosing of compounds via oral gavage was carried out for two weeks. Vehicle treated and naïve animals were used as controls. Clinical score and joint swelling (hind limb volume) were measured on Day 0, before the first dosing and then 3 times a week until the end of the study. Body weight measurements were performed thrice weekly to assess compound tolerability. Criteria (on a scale of 0-4 per limb) were as follows: 0, No evidence of erythema and swelling; 1, Erythema and mild swelling confined to the mid-foot (tarsals) or ankle joint; 2, Erythema and mild swelling extending from the ankle to the mid-foot; 3, Erythema and moderate swelling extending from the ankle to the metatarsal joints; 4, Erythema and severe swelling encompass the ankle, foot, and digits. Following 2-3 weeks of dosing, representative animals were bled at different time intervals over a 0-24 h time-period to assess compound exposure levels in the plasma via LC/MS.

For isolation of synovial fluid prior to study termination, animals were anesthetized and the skin on the hind limbs were separated to ensure exposure of knee joint. A small incision was made near the top of the knee joint and the articular cavity was rinsed with 60 μl phosphate-buffered saline (PBS) for 2 time and ˜50-60 μl synovial fluid was collected. The synovial fluid samples were spun down, and the supernatant stored at −80° C. prior to cytokine analysis. At study termination, plasma was prepared from whole blood and stored at −80° C. prior to cytokine analysis and measurement of total and anti-collagen IgG. Single cell suspensions were prepared from harvested spleens following standard methods and Treg frequencies measured by flow cytometry-based immunophenotyping of freshly isolated cells.

### Immunophenotyping

Harvested spleens were homogenized in cold PBS and cellular debris removed by passing through a 70 μM cell strainer. The cell suspension was centrifuged at 300×g for 5 min at 4° C. and the pellet was treated with 1×RBC lysis buffer per manufacturer's protocol (Thermofisher). The washed cell pellet was resuspended for a final concentration of 5-10×10⁶ cells/ml and 100 μl of cell suspension was processed for immunophenotyping. Briefly, cells were first stained with a Fixable Live-Dead dye (BD Biosciences, Franklin Park, NJ) followed by FcR blocking with a rat anti-CD32 antibody (BD) per manufacturer's protocol. Cells were then stained with a panel of fluorescently labeled antibodies (BD, unless otherwise mentioned) targeting rat immune cell surface markers, that included CD45 (clone OX-1, pan lymphocyte marker), CD3 (clone 1F4,T-cell), CD45RA (clone OX-33, B-cell), CD11b/c (clone OX-42, monocyte/macrophages), CD4 (clone OX-35, helper T-cells), CD8 (clone OX-8, cytotoxic T-cells), CD25 (clone OX-39, high affinity IL-2 receptor α), for 30 min on ice. Cells were washed with cold FACS wash buffer (BD) and fixed with 1×FoxP3/Transcription Factor Fixation Buffer (Thermofisher) for overnight at 4° C. Cells were subsequently permeabilized by washing with 1×FoxP3/Transcription Factor Permeabilization and Wash Buffer (Thermofisher) followed by staining for intracellular FoxP3 (clone 150D, Biolegend, San Diego, CA), a canonical Treg marker, for 30 min. Cells were washed 2-3× with the permeabilization and wash buffer as mentioned earlier, and finally resuspended in FACS wash buffer. Samples were acquired on a BD LSRFortessa flow cytometer and data analyzed using the FlowJo software (BD).

### Cytokine Analysis

All cytokine measurements were performed using commercially available kits and as per manufacturer's protocol. Supernatants from human whole blood and cellular assays were quantified using the human Proinflammatory Panel 1 (human) kit on a Sector Imager 6000 reader (Meso Scale Discovery, Rockville, MD). IL-17a was quantified using the V-Plex Human IL-17a kit on a Sector Imager 6000 reader (Meso Scale Discovery, Rockville, MD). Supernatants from rat whole blood assay were analyzed for IL-2 levels using the rat IL-2 Duoset ELISA kit (R&D). Plasma and synovial samples harvested from the rat CIA study were quantified for proinflammatory cytokines using the rat V-PLEX Proinflammatory Panel 2 kit (MSD).

### Anti-Collagen Antibody and Total IgG

Anti-collagen antibodies in rat CIA plasma were analyzed using the Rat anti-bovine Type II Collagen IgG Antibody ELISA Kit (Chondrex). The assay was performed according to the manufacturer's protocol, with samples diluted 1:100,000. Total IgG antibodies in rat plasma were analyzed using the Rat total IgG Uncoated ELISA Kit (Invitrogen, Waltham, MA). The assay was performed according to the manufacturer's protocol, with samples diluted 1:400,000.

nTreg Isolation, Expansion, and In Vitro Suppression Assay

Natural nTregs (nTreg) were isolated from human donor PBMCs Human CD4⁺CD127^{low}CD25⁺ Regulatory T-Cell Isolation Kit (Stemcell) and expanded in culture. Briefly, nTregs were activated with Dynabeads Human T-Activator CD3/CD28 (Thermofisher) at a 1:1 bead to cell ratio in complete RPMI media supplemented with 10% FBS, 10 mM HEPES, 2 mM GlutaMAX, 1 mM Na-Pyruvate, and 1×MEM non-essential amino acids (Thermofisher). After 2 days in culture, the culture volume was doubled, and IL-2 was added at a final concentration of 300 IU (Peprotech). At days 5 and 7, cells were expanded in the presence of 300 IU IL-2. On day 9, cells were restimulated with Dynabeads at a 1:1 bead to cell ratio. On day 13, nTregs were harvested and beads were magnetically removed for downstream Treg suppression assays.

For the in vitro Treg suppression assay, naïve CD4⁺ T-cell proliferation was assessed by measuring Cell Trace Violet (CTV) (Thermofisher) dilution in the presence of varying ratios of autologous nTregs. In brief, naïve CD4⁺ T-cells were isolated from human donor PBMCs using the Human Naïve CD4⁺ T-cell Isolation Kit (Stemcell) and labeled with CTV according to manufacturer's protocol. Naïve CD4⁺ T-cells were activated with Dynabeads Human T-Activator CD3/CD28 beads at a 1:8 bead to cell ratio for 3 days in the presence of varying ratios of MALT1i-treated nTregs in complete RPMI. Cells were then processed for flow analysis of proliferation. Briefly, cells were first stained with a Fixable Live-Dead dye (BD) and then stained with the following panel of fluorescently labeled antibodies (Biolegend, unless otherwise mentioned): CD4 (clone RPA-T4) and CD25 (clone M-A251), for 20 min at 4° C. Cells were subsequently stained for intracellular FoxP3 (clone 236A/E7, Invitrogen) using the Foxp3 Transcription Factor Staining Buffer Set (eBiosciences) according to the manufacturer's instructions. Cells were analyzed on a BD LSRFortessa flow cytometer. Percent suppression was calculated using the following formula: % suppression=((% naïve T-cell proliferating)−(% naïve T-cell+Treg proliferating))/(% naïve T-cell proliferating)×100.

pSTAT5 Measurement in nTregs

Purified Tregs were expanded as indicated above. On day 12, cells were harvested and incubated with MALT1i at various concentrations for 18 h in serum-free RPMI at 37° C., 5% CO₂. Tregs were then stimulated with 25 IU IL-2 (Peprotech) for 15 min, followed by fixation with 2% paraformaldehyde. Cells were then permeabilized with 90% methanol and stained with an anti-pSTAT5 antibody (Y694, clone 47, BD) at a 1:100 dilution. Levels of pSTAT5 were analyzed on a BD LSRFortessa flow cytometer.

### MALT1 Biochemical Assay

Inhibitor potency was evaluated by measuring enzymatic activity of full length MALT1 at varying concentrations of compound. The enzymatic assay consists of a single substrate reaction that monitors the release of a fluorescent dye upon cleavage of the peptide substrate. The peptide substrate has the following sequence: Ac-Leu-Arg-Ser-Arg-Rh110-dPro (custom synthesis from WuXi AppTec, Shanghai, China). The assay buffer consists of 50 mM Hepes, pH 7.5, 0.8 M sodium citrate, 1 mM DTT, 0.004% tween-20, and 0.005% bovine serum albumin (BSA). Steady-state kinetic analysis of peptide substrate binding resulted in a Michaelis-Menten constant (K_{M}) of 150 μM. The assay was performed in a 384-well F-bottom polypropylene, black microplate (Greiner Bio_One, Catalog no. 781209) at 15 nM enzyme and 30 μM peptide substrate. The reaction was quenched after 60 minutes with the addition of iodoacetate at a final concentration of 10 mM. Total fluorescence was measured using an Envision (PerkinElmer) with fluorescence excitation at 485 nm and emission at 520 nm.

For potency determination, 1 μL of serially diluted compound (in 100% DMSO) was pre-incubated with 40 μL of enzyme for 30 minutes. The reaction was initiated with the addition of 10 μL of peptide substrate. The relative fluorescence units were transformed to percent inhibition by using 0% and 100% inhibition controls as reference. The 100% inhibition control consisted of 1 μM final concentration of(S)-1-(5-chloro-6-(2H-1,2,3-triazol-2-yl) pyridin-3-yl)-3-(2-chloro-7-(1-methoxyethyl) pyrazolo[1,5-a]pyrimidin-6-yl) urea (IC₅₀=15 nM), while the 0% inhibition control consisted of 2% DMSO. IC₅₀ values were calculated by fitting the concentration-response curves to a four-parameter logistic equation in GraphPad Prism.

### Immunoblotting Analysis of MALT1 Function

Primary human CD4⁺ T cells were isolated from human donor PBMCs by negative selection (EasySep Human CD4⁺ T cell isolation kit, Stemcell Technologies). T cells were pre-incubated with 0-30 μM of MALT1i, or DMSO as a control, for 30 min prior to stimulation with 50 ng/mL phorbol 12-myristate 13-acetate (Sigma) and 1.34 mM ionomycin (Sigma). Whole cell lysates were prepared in lysis buffer (CellLyticMT Cell Lysis reagent (Sigma)) with HALT protease and phosphatase inhibitors (ThermoFisher Scientific) and 10 nM MG-132 (Sigma). Proteins were separated on NuPAGE 4-12% Bis-Tris denaturing gels (Invitrogen, Waltham, MA) and transferred onto nitrocellulose membranes (iBLOT2, Invitrogen). After blocking for 1 h at room temperature, blots were incubated overnight in primary antibodies, washed with PBS+0.05% Tween-20, incubated for 2 h in secondary antibodies, washed and imaged using a LI-COR Odyssey infrared imager (LI-COR Biosciences, Lincoln, NE). Antibodies were diluted in Intercept TBS-T blocking buffer (LI-COR). Bands were quantified using ImageStudio software (LI-COR Biosciences). Primary antibodies used were: anti-HOIL-1 (Millipore Sigma, MABC576), anti-BCL10 (Abcam, 33905), anti-pIKKab (Cell Signaling Technologies, 2078), anti-pJNK (Cell Signaling Technologies, 4668), anti-COX-IV (Cell Signaling Technologies, 4668). Secondary antibodies used were: anti-mouse IgG H+L DyLight 680 (Cell Signaling Technologies, 5470), anti-rabbit IgG H+L DyLight 800 (Cell Signaling Technologies, 5151).

### Statistics

All statistical calculations were performed using Graphpad Prism 9.2.0. Datapoints from most readouts, e.g., cytokine expression, cell proliferation, disease scores, antibody levels, etc. were expressed as mean±standard error of mean (S.E.M). Significant differences (p<0.05) across unpaired observations were calculated using one-way ANOVA and corrected for multiple comparisons using Dunnett's test. Comparison between two groups (e.g., healthy vs arthritic animals) were performed using a Mann-Whitney test. Cytokine expression data in treatment groups were expressed as a percent of DMSO control normalized to 100% activity. Potency measurements, IC50 and IC90 values, were performed using a 4-parameter fit in GraphPad Prism. Pharmacokinetic-pharmacodynamic (PK-PD) correlations were expressed by similarly fitting plasma concentrations of MALT1i over a 24 h period since last dose (AUC₀₋₂₄ₕ) from individual subjects to the corresponding effects observed with regards to reduction in clinical scores and/or splenic Tregs.

### Example 2: Pharmacological Modulation of MALT1 Dampens Multiple Immune Effector Functions in Primary Human Cells

The impact of MALT1i (REO-MALT1 inhibitor) was evaluated in inflammatory processes associated with adaptive and innate immune cells activated via stimulation of distinct ITAM-containing immunoreceptors (FIGS. 3B-3E and Table 2). MALT1i inhibited the release of proinflammatory cytokines (IFNγ, IL-2 and TNFα) and IL-17A from CD45RO⁺ memory T-cells activated in vitro by α-CD3/-CD28/-CD2 (FIGS. 3B and 3C, respectively) in a dose-dependent manner. MALT1i also attenuated B-cell proliferation stimulated via co-crosslinking of BCR/CD40L with anti-human IgA/IgG/IgM and recombinant CD40L (FIG. 3D). In myeloid cells, MALT1i effectively suppressed proinflammatory cytokine expression from human monocyte-derived macrophages differentiated in vitro and stimulated via FcγR with anti-human IgG/human IgG-derived immune complexes (FIG. 3E and Table 2). Macrophages stimulated with bacterial lipopolysaccharide (LPS), which does not signal through an ITAM-dependent mechanism, were insensitive to MALT1i. By contrast, macrophages stimulated with depleted zymosan, which signals through the ITAM-dependent Dectin-1 receptor, were sensitive to MALT1 inhibition. Taken together, these data show that inhibition of MALT1 with an allosteric inhibitor can selectively suppress ITAM-driven autoimmune inflammatory processes by multiple immune cell types in a stimulus-restricted manner.

TABLE 2
Determination of MALTli potency in in vitro cellular assays

| | | | | Effect size |
|---|---|---|---|---|
| | | | | (% Max. |
| Cells/(stimulation) | Readout | IC₅₀ (mM) | IC₉₀ (mM) | inhibition) |
| Jurkat-T cells | IL-2 | 0.03 ± 0.002 | 0.20 ± 0.01 | >95 |
| (a-CD3/-CD28/-CD2) | | | | |
| CD4⁺CD45RO⁺ Memory T- | IL-2 | 0.05 ± 0.01 | 0.31 ± 0.07 | >90 |
| cells (a-CD3/-CD28/-CD2) | IFNg | 0.09 ± 0.02 | 1.49 ± 0.52 | ~80 |
| | TNFa | 0.09 ± 0.04 | 0.80 ± 0.16 | ~85 |
| CD4⁺ Total T-cells | IL-2 | 0.03 ± 0.009 | 0.17 ± 0.003 | >95 |
| (a-CD3/-CD28) | IFNg | 0.13 ± 0.06 | 0.37 ± 0.08 | >95 |
| | TNFa | 0.05 ± 0.008 | 0.09 ± 0.002 | ~85 |
| CD4⁺CD45RO⁺ Memory T- | IL-17 | 0.13 ± 0.02 | 1.71 ± 0.36 | >90 |
| cells | | | | |
| (a-CD3/-CD28/-CD2) | | | | |
| Total B cells (BCR/CD40L) | Proliferation | 0.06 ± 0.004 | 0.45 ± 0.13 | ~60 |
| Monocytes à Macrophages | TNFa | 0.07 ± 0.02 | 0.32 ± 0.11 | >75 |
| (depleted zymosan) | IL-6 | 0.06 ± 0.07 | 0.25 ± 0.21 | >60 |
| Monocytes à Macrophages | TNFa | 0.09 ± 0.01 | 0.35 ± 0.16 | ~74 |
| (aIgG + IgG, immune- | IL-6 | 0.05 ± 0.03 | 0.28 ± 0.07 | >75 |
| complex) | | | | |

BCR = B-cell receptor.
Values plotted are Mean ± S.E.M from assays performed twice with 1-3 donors/animals. Potency values determined using Graphpad Prism software.

In order to understand the dose-dependent relationship between MALT1i efficacy and Treg effects, a surrogate measure of in vivo target engagement was developed using a whole blood assay. In rat and human whole blood, spike-in of MALT1i was followed by TCR stimulation (a MALT1-dependent pathway) and measurement of cytokine secretion. This assay takes into account plasma protein binding and enables understanding of the potency of the inhibitor in a complex mixture of cells. As shown in Table 3, pretreatment of human whole blood with MALT1i and subsequent co-stimulation of the TCR with antibodies directed to CD3 and CD28, or PMA/ionomycin (surrogate ITAM signaling, data not shown), resulted in suppression of proinflammatory cytokine production. While several cytokines, including IL-2, IFNγ and TNFα, were found to be impacted, IL-2 was shown to be most sensitive to MALT1 inhibition. The IL-2 response with PMA/ionomycin showed a similar IC₅₀ compared to anti-CD3/28 stimulation.

TABLE 3
Determination of MALT1i potency in whole blood

| | | | | Effect Size |
|---|---|---|---|---|
| | | | | (% Max. |
| Stimulus | Cytokine | IC₅₀ (μM) | IC₉₀ (μM) | inhibition) |
| Human | IL-2 | 0.27 ± 0.01 | 1.39 ± 0.60 | |
| α-CD3/-CD28 | IFNγ | 0.79 ± 0.13 | 5.38 ± 0.76 | >99 |
| | TNFα | 0.59 ± 0.19 | 3.83 ± 0.38 | |
| Rat | | | | |
| PMA/ionomycin | IL-2 | 0.18 ± 0.02 | 1.90 ± 0.29 | 70 |
| | | | (est.) | |

PMA = phorbol 12-myristate 13-acetate; est. = estimated.
Values plotted are Mean ± S.E.M from assays performed twice with 1-3 donors/animals. Potency values determined using Graphpad Prism software.

The relationship between the cytokine PD effect and MALTi target engagement was next assessed by measuring the dose-dependent effects of MALT1i on proteolytic cleavage of a known MALT1 substrate. Using immunoblotting, cleavage of the MALT1 substrate HOIL-1 was monitored across a range of MALT1i concentrations, encompassing 50-90% of target coverage as determined using the human whole blood assay. At a compound concentration equivalent to human whole blood IL-2 IC50, protease function was impacted by >80% (FIG. 3F).

### Example 3: Treatment with MALT1i Ameliorates Disease Pathogenesis in Rat Collagen Induced Arthritis

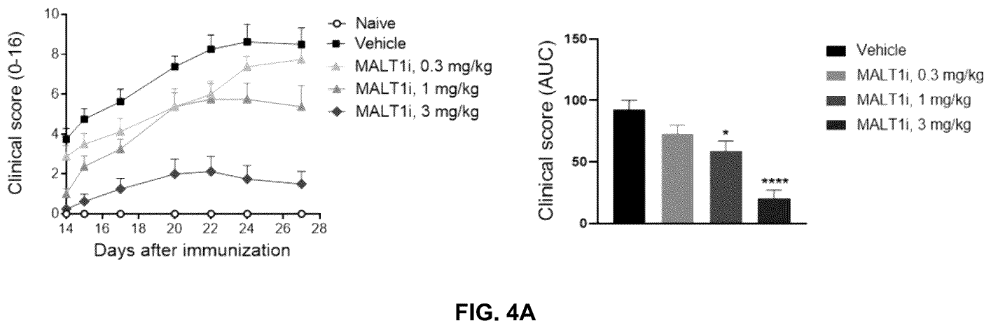

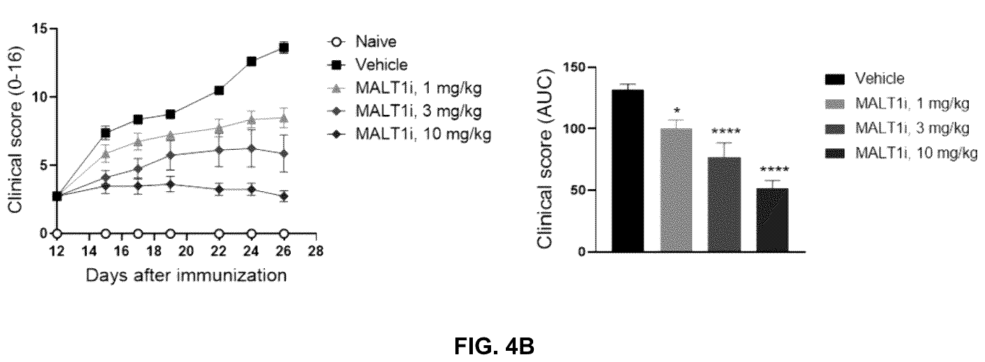

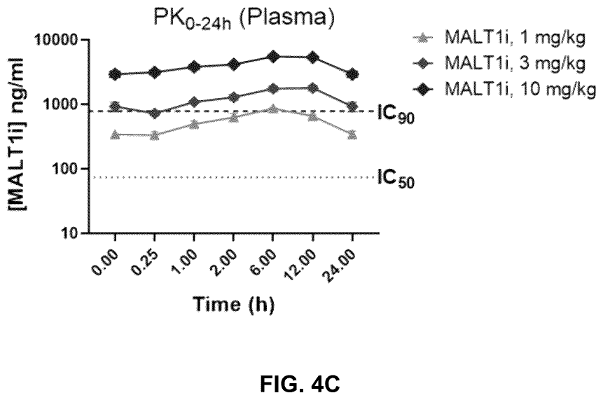

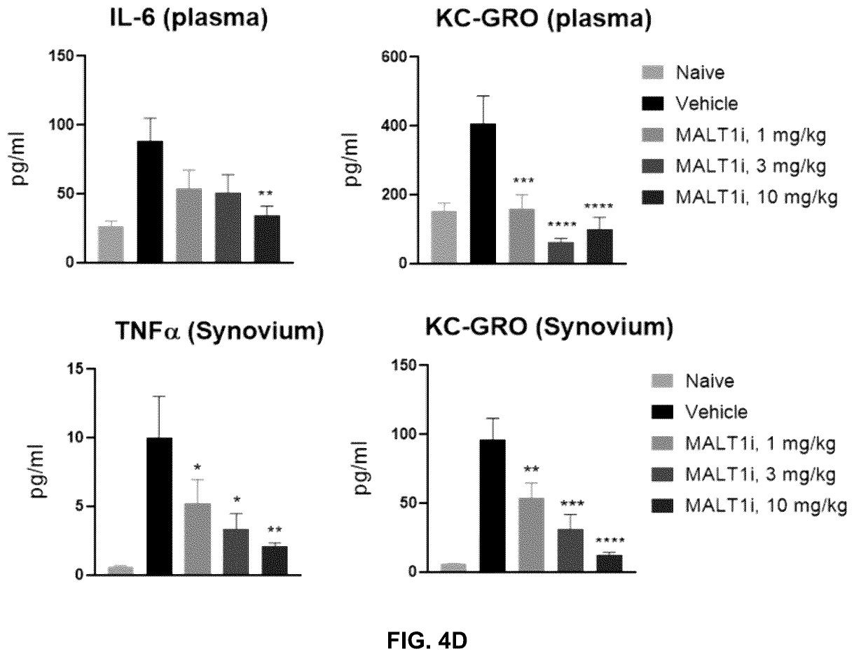

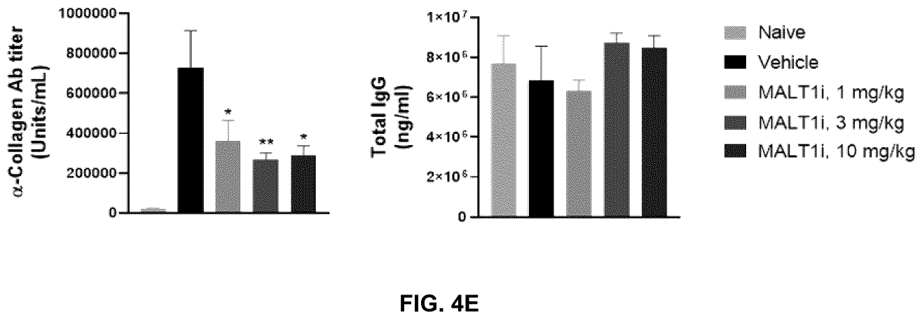

The effect of MALTi administration in a rat CIA model was assessed. Oral administration of MALT1i demonstrated a dose-dependent decrease in disease activity scores in both prophylactic (FIG. 4A) and therapeutic (FIG. 4B) dosing regimens, as shown by reduction in clinical score over time and lower overall disease burden. Therapeutic MALT1i administration dose dependently and significantly reduced disease scores at all doses tested with ≥50% reduction observed with the 3 and 10 mg/kg doses. Analysis of MALT1i levels in the plasma of compound-treated animals indicated that MALT1i is efficacious at concentrations that result in 90% target coverage (rat whole blood IC₉₀, Table 3) for approximately 10 h over a 24 h dosing period (FIG. 4C).

The effector phase in the rat CIA model is characterized by systemic inflammatory responses and by inflammation in the joints caused by neutrophil accumulation and deposition of antigen-antibody immune complexes with resultant activation of the complement cascade. Proinflammatory cytokines (e.g., TNFα, IL-1β) secreted by activated macrophages are also implicated in sustaining inflammation.

At study termination, the highest dose of MALT1i significantly reduced IL-6 levels in the plasma. Significant inhibition of KC-GRO, a neutrophil chemoattractant produced by activated macrophages, was also observed in both the plasma and the synovial fluid at all doses tested. Significant reduction of TNFα in the synovial fluid was also observed at all doses (FIG. 4D). Furthermore, analysis of the plasma for anti-collagen antibodies, which are implicated in immune-complex formation, showed significant reduction with MALT1i treatment (FIG. 4E). There was no effect on the total IgG levels across all treatment groups when compared with naïve animals, suggesting this level of target coverage does not lead to complete immunosuppression, although more systemic effects will need to be defined in subsequent studies. These data indicate that maintaining a systemic MALT1i exposure equivalent to IC90 coverage over the full 24 h period is not necessary to suppress known disease drivers of chronic inflammation and suggested lack of broad immunosuppression.

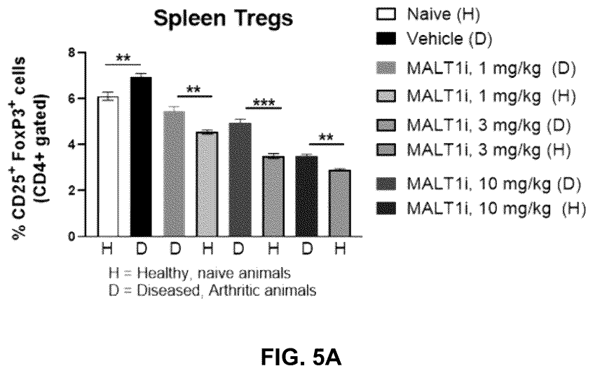

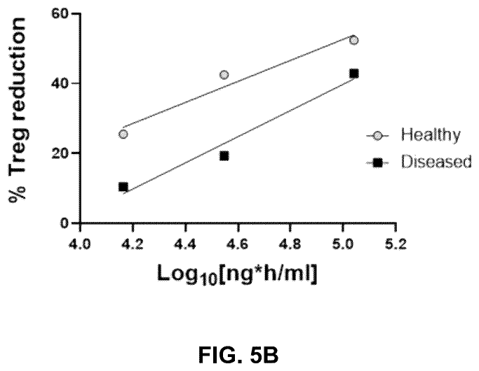

Next, the effects of the MALT1i on the peripheral Treg compartment on naïve and CIA rats with active disease were compared. To match the dosing paradigm of the CIA model, healthy rats were treated for 14 days with MALT1i, and Tregs were measured contemporaneously in healthy rats and in CIA rats treated with the same three doses of MALT1i. Treg numbers were slightly elevated in the context of disease and that reductions in Treg numbers in the spleens of MALT1i-treated rats were significantly more reduced at any given dose than Treg numbers from spleens of rats with active CIA (FIG. 5A). This observation was further confirmed with an exposure-response analysis, which showed that at similar plasma drug concentrations, the effect of Treg reduction was more pronounced in naïve animals compared with diseased animals (FIG. 5B).

### Example 4: Pharmacokinetics and Pharmacodynamics of MALT1 Inhibitors

Effects of MALT1 inhibitors were analyzed to determine if efficacy could be achieved without impacting the Treg compartment at efficacious concentrations of MALT1i. In this example, MALT1 inhibitors, including REO-981 MALT1 inhibitors, were administered to rats in a collagen induced arthritis (CIA) rat model and analyzed according to the methods described in Example 1.

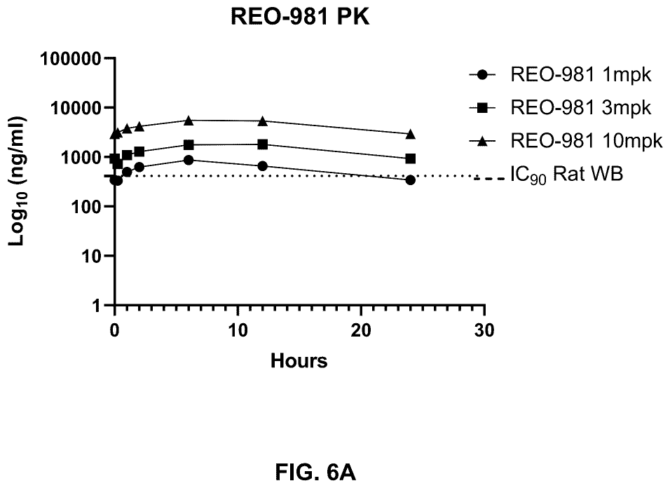

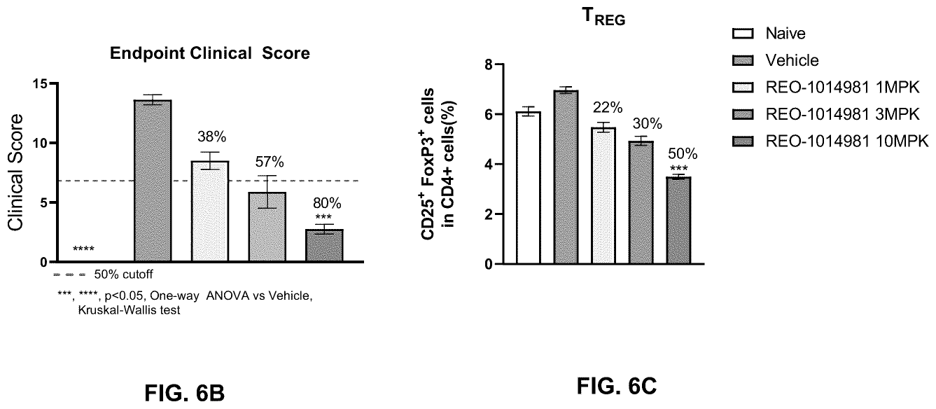

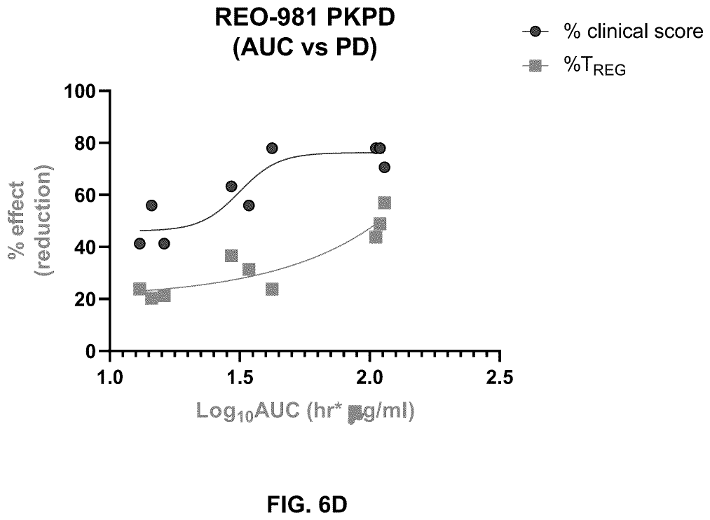

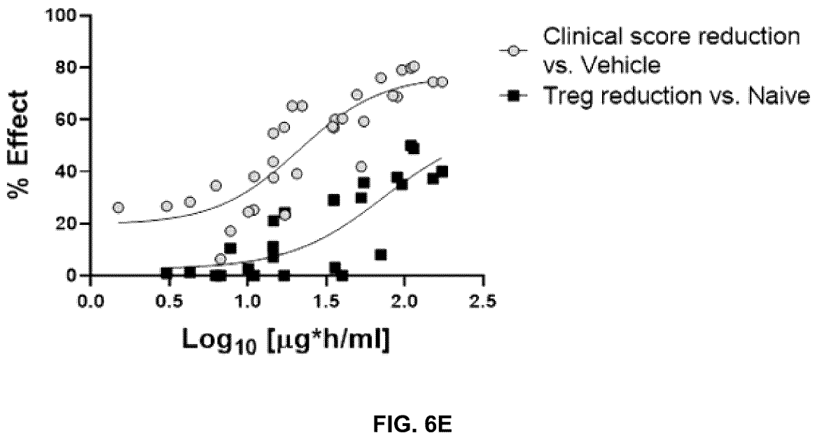

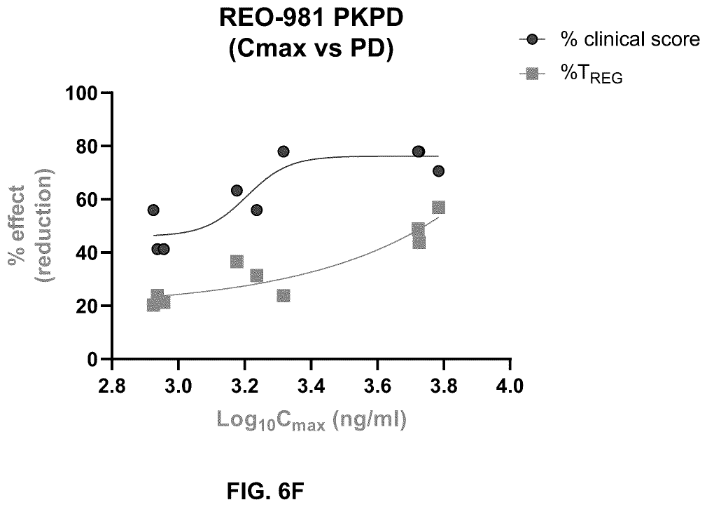

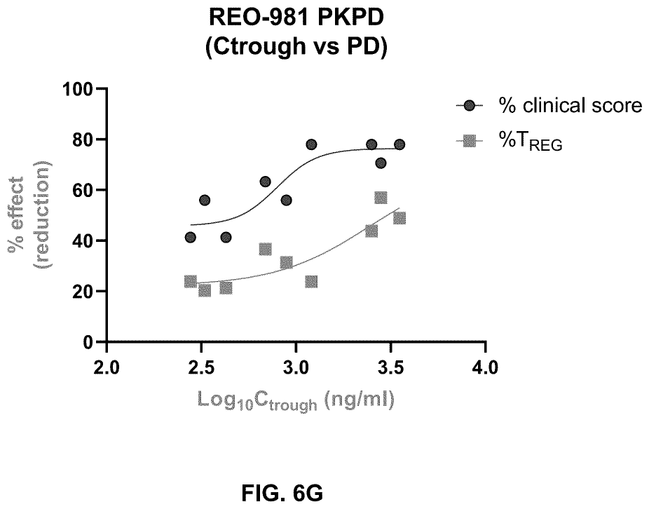

FIG. 6A depicts single dose pharmacokinetics of the REO-981 MALT1 inhibitor in a rat collagen induced arthritis (CIA) model. The REO-981 MALT1 inhibitor was administered in 3 doses (1 mg/kg (mpk), 3 mg/kg (mpk), and 10 mg/kg (mpk)). FIG. 6A shows the blood PK of the REO-981 MALT1 inhibitor up to 24 hours post-administration. Furthermore, FIG. 6A shows the IC90 value (see horizontal dotted line). Here, rats administered 3 mg/kg or 10 mg/kg REO-981 MALT1 inhibitor exhibited blood concentration levels of the MALT1 inhibitor that was above the IC90 value for all 24 hours. Additionally, rats administered the low dose of 1 mg/kg REO-981 MALT1 inhibitor also exhibited blood concentration levels of the MALT1 inhibitor that was above the IC90 value for more than 20 of the 24 hours.

FIG. 6B depicts endpoint clinical score at various doses of the REO-981 MALT1 inhibitor in a rat collagen induced arthritis (CIA) model. Here, a 1 mg/kg dose of REO-981 resulted in a 38% reduction in the clinical score, a 3 mg/kg dose of REO-981 resulted in a 57% reduction in the clinical score, and a 10 mg/kg dose of REO-981 resulted in a 80% reduction in the clinical score (reduction in comparison to the vehicle treatment).

FIG. 6C depicts levels of Tregs following administration of REO-981 MALT1 inhibitor in a rat collagen induced arthritis (CIA) model. Here, a 1 mg/kg dose of REO-981 resulted in a 22% reduction in the Treg levels, a 3 mg/kg dose of REO-981 resulted in a 30% reduction in the Treg levels, and a 10 mg/kg dose of REO-981 resulted in a 50% reduction in the Treg levels (reduced in comparison to vehicle).

FIG. 6D depicts clinical score and percent reduction in Tregs following administration of REO-981 MALT1 inhibitor as a function of area under the curve (AUC). Here, FIG. 6D shows the decoupling of the efficacy of the REO-981 MALT1 inhibitor and the reduction of Treg levels. An exposure-response analysis revealed an uncoupling of efficacy in CIA from Treg reduction (FIG. 4D). Specifically, a drug concentration AUC of 31,500 ng*hr/ml achieved a 50% effect on disease score while a drug concentration AUC of 155,000 ng*hr/ml reduced Treg numbers by 50% compared to naïve animals (FIG. 6D). Put another way, as shown in FIG. 6D, at about a log₁₀AUC value of 1.5 μg*hr/mL, the REO-981 MALT1 inhibitor exhibits efficacy in the form of ˜50% reduction in the clinical score. Furthermore, at about a log₁₀AUC value of 1.9 μg*hr/mL, the REO-981 MALT1 inhibitor exhibits reduction of Tregs in the form of ˜40% reduction in Treg levels. Thus, as an example, a log₁₀AUC range between 1.5 μg*hr/mL and 1.9 μg*hr/mL represents a range in which the REO-981 MALT1 inhibitor achieves a decoupling of the efficacy of the MALT1 inhibitor and the reduction of Treg levels.

To confirm that this uncoupling of efficacy from Treg reduction was not a function of the specific compound tested, exposure-responses from four distinct MALT1 inhibitors were further analyzed. FIG. 6E shows combined data using 4 structurally distinct MALT1 inhibitors, which depicts uncoupling of efficacy and Treg reduction. Generally, the drug concentrations required to achieve a significant effect on efficacy were 3-5× lower than the drug concentrations required for reduction in Treg numbers (FIG. 6E) indicating that uncoupling of efficacy in CIA from reductions in Tregs is a generalizable feature of allosteric inhibition of MALT1.

FIG. 6F depicts clinical score and percent reduction in Tregs following administration of REO-981 MALT1 inhibitor as a function of Cmax. As shown in FIG. 6F, at about a log₁₀Cmax value of 3.1 ng/mL, the REO-981 MALT1 inhibitor exhibits efficacy in the form of ˜50% reduction in the clinical score. Furthermore, at about a log₁₀Cmax value of 3.6 ng/ml, the REO-981 MALT1 inhibitor exhibits reduction of Tregs in the form of ˜40% reduction in Treg levels. Thus, as an example, a log₁₀Cmax range between 3.1 ng/ml and 3.6 ng/mL represents a range in which the REO-981 MALT1 inhibitor achieves a decoupling of the efficacy of the MALT1 inhibitor and the reduction of Treg levels.

FIG. 6G depicts clinical score and percent reduction in Tregs following administration of REO-981 MALT1 inhibitor as a function of Ctrough (trough concentration). As shown in FIG. 6G, at about a log₁₀Ctrough value of 2.6 ng/ml, the REO-981 MALT1 inhibitor exhibits efficacy in the form of ˜50% reduction in the clinical score. Furthermore, at about a log₁₀Ctrough value of 3.3 ng/mL, the REO-981 MALT1 inhibitor exhibits reduction of Tregs in the form of ˜40% reduction in Treg levels. Thus, as an example, a log₁₀Ctrough range between 2.6 ng/mL and 3.3 ng/mL represents a range in which the REO-981 MALT1 inhibitor achieves a decoupling of the efficacy of the MALT1 inhibitor and the reduction of Treg levels.

Altogether, in this example, the impact of REO-981 MATLli on splenic Treg numbers in the CIA model was analyzed to test the impact of MALT1i on Tregs in disease. A statistically significant decrease of CD25 FoxP3 Tregs only in rats that receive the highest dose of MALT1i. This was in contrast to disease scores, and to synovial and plasma cytokine and chemokine concentrations, where significant reduction in disease scores was observed at all doses tested. There was further evidence of uncoupling of efficacy from Treg reduction when plasma drug concentrations over time (AUC) were plotted against effects on clinical score and Treg numbers (FIG. 6D). There was a clear separation in the two measured effects: an AUC of 31,500 ng*h/ml resulted in a 50% reduction in disease score while an AUC of 155,000 ng*h/ml was required for a 50% reduction in Treg numbers. These data show that efficacy was achieved at concentrations of MALT1i that were ˜5-fold lower than concentrations that reduced Tregs. Additional evidence of uncoupling was observed in the sensitivity of Treg reduction to MALT1 inhibitor treatment in disease versus naïve rats. It was observed that naïve animals had greater magnitude of Treg reduction at any given dose compared to diseased animals. This held true when drug exposure was measured. At any given unit of exposure, there was a greater impact on Treg numbers in healthy animals compared to disease animals thus eliminating the possibility that the dose differences were the consequence of altered drug exposure.

There may be different mechanistic explanations for the uncoupling of efficacy and Treg reduction. Analysis of drug target coverage can provide insight into mechanisms of uncoupling. The inhibition of IL-2 can be used as a marker for target engagement as it is tightly linked to MALT1 signaling and may be qualified as a proximal pharmacodynamic event following modulation of the target. The amount of target coverage needed for efficacy with MALT1i tested was equivalent to, or less than, the IL-2 IC₉₀ over the 24 h dosing period (FIG. 6C). Analyses of target coverage for multiple other compounds indicate that the time exceeding the IC₉₀ over the dosing period to achieve efficacy ranged from 4 to 12 h (data not shown). The ability of MALT1i to impact multiple pathogenic factors (e.g., TNFα, IL-6, KC-GRO) may underlie the observed efficacy in the absence of a full target coverage over a 24 h period. Thus, the resulting additive effect may translate to a requirement for less target coverage (e.g., IC₅₀ concentrations of multiple cellular effects) to achieve a significant effect on disease score. By contrast, the impact on Tregs could be due to inhibition of only a single factor, such as IL-2, which may not be completely affected at efficacious doses. Consistent with this hypothesis, reduction in Treg numbers required target coverage exceeding the IL-2 IC₉₀ for the full dosing period regardless of the compound used. Thus, a dosing paradigm where MALT1 is not fully inhibited for the duration of the dosing interval may spare enough MALT1-driven IL-2 to maintain Tregs, but not enough to expand pathogenic effector T cell populations, as would be predicted by the increased sensitivity of Tregs to lower concentrations of IL-2. When the systemic drug concentration exceeds the IC₉₀ for an extended period of time, e.g., >24 h (FIG. 6C), IL-2 may become limiting and impact Treg maintenance. This would also explain the difference in sensitivity of Treg reduction in naïve compared with inflamed animals.

### Example 5: Additional Pharmacokinetics and Pharmacodynamics of REO-528 and REO-703 MALT1 Inhibitors

In this example, MALT1 inhibitors, including those of REO-528, REO-538, REO-076, and REO-703, were administered to rats in a collagen induced arthritis (CIA) rat model, were administered to rats in a collagen induced arthritis (CIA) rat model and analyzed according to the methods described in Example 1.

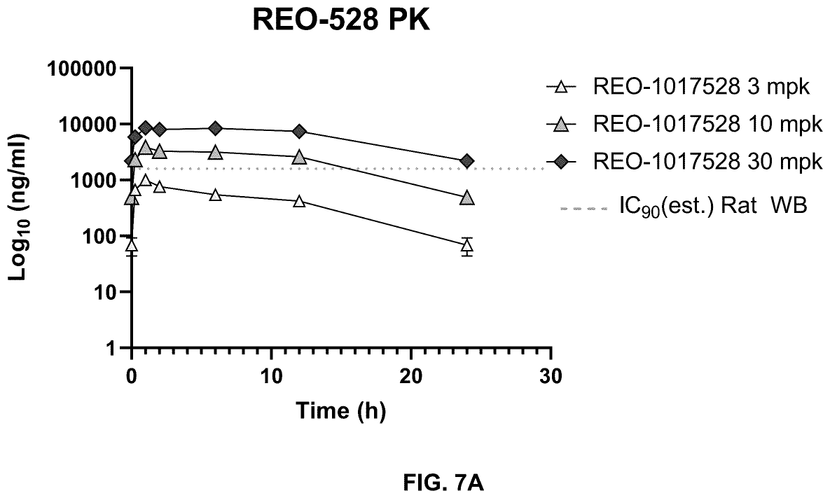

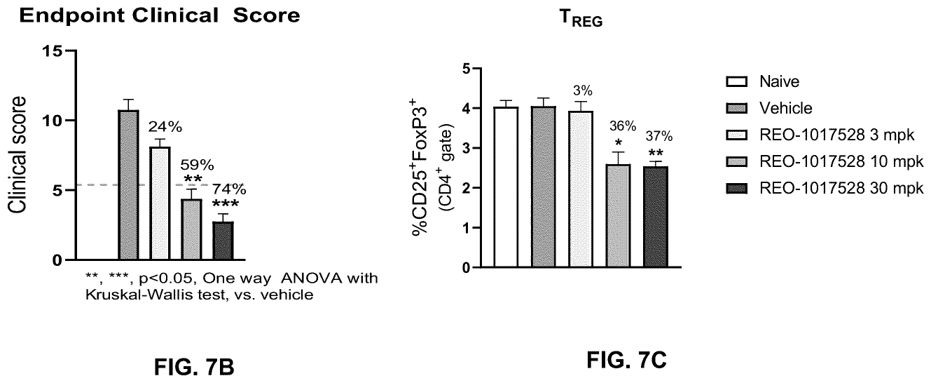

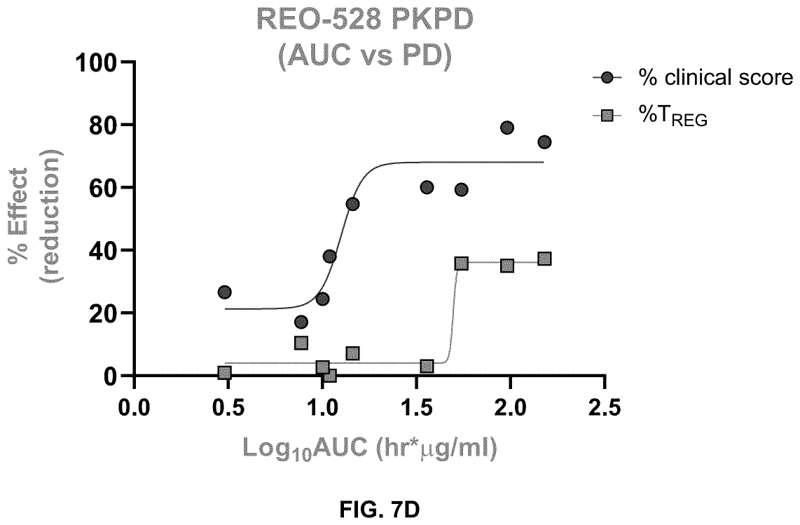

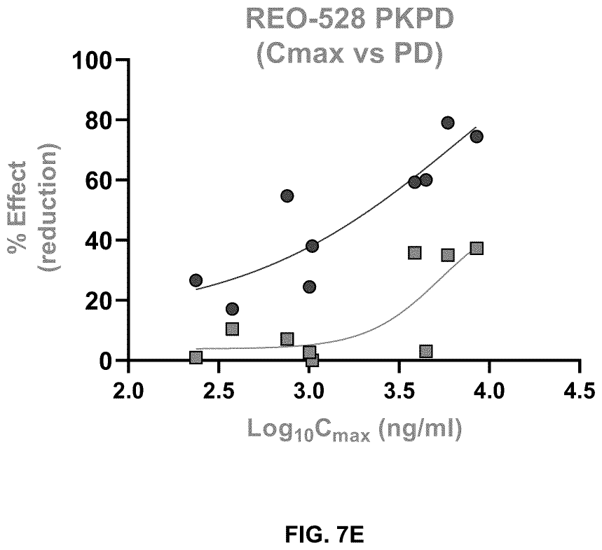

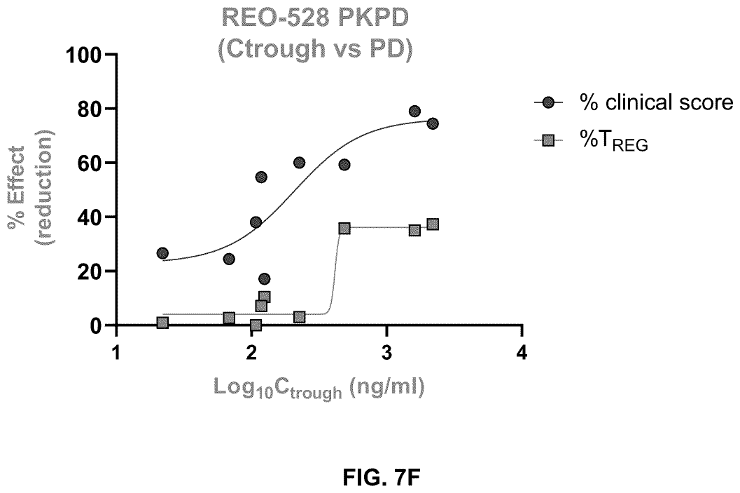

FIG. 7A depicts single dose pharmacokinetics of the REO-528 MALT1 inhibitor in a rat collagen induced arthritis (CIA) model. The REO-528 MALT1 inhibitor was administered in 3 doses (3 mg/kg (mpk), 10 mg/kg (mpk), and 30 mg/kg (mpk)). FIG. 7A shows the blood PK of the REO-528 MALT1 inhibitor up to 24 hours post-administration. Furthermore, FIG. 7A shows the estimated IC90 value (see horizontal dotted line). Here, rats administered 30 mg/kg REO-528 MALT1 inhibitor exhibited blood concentration levels of the MALT1 inhibitor that was above the IC90 value for all 24 hours. Additionally, rats administered the dose of 10 mg/kg REO-528 MALT1 inhibitor exhibited blood concentration levels of the MALT1 inhibitor that was above the IC90 value for more than 12 of the 24 hours.

FIG. 7B depicts endpoint clinical score at various doses of the REO-528 MALT1 inhibitor in a rat collagen induced arthritis (CIA) model. The dotted line in FIG. 7B indicates the clinical score corresponding to 50% symptom reduction. Here, a 3 mg/kg dose of REO-528 resulted in a 24% reduction in the clinical score, a 10 mg/kg dose of REO-528 resulted in a 59% reduction in the clinical score, and a 30 mg/kg dose of REO-528 resulted in a 74% reduction in the clinical score (reduction in comparison to the vehicle treatment).

FIG. 7C depicts levels of Tregs following administration of REO-528 MALT1 inhibitor in a rat collagen induced arthritis (CIA) model. Here, a 3 mg/kg dose of REO-528 resulted in a 3% reduction in the Treg levels, a 10 mg/kg dose of REO-528 resulted in a 36% reduction in the Treg levels, and a 30 mg/kg dose of REO-528 resulted in a 37% reduction in the Treg levels (reduced in comparison to vehicle).

FIG. 7D depicts clinical score and percent reduction in Tregs following administration of REO-528 MALT1 inhibitor as a function of area under the curve (AUC). Here, FIG. 7D shows the decoupling of the efficacy of the REO-528 MALT1 inhibitor and the reduction of Treg levels. As shown in FIG. 7D, at about a log₁₀AUC value of 1.2 μg*hr/mL, the REO-528 MALT1 inhibitor exhibits efficacy in the form of ˜50% reduction in the clinical score. Furthermore, at about a log₁₀AUC value of 1.6 μg*hr/mL, the REO-528 MALT1 inhibitor exhibits reduction of Tregs in the form of ˜40% reduction in Treg levels. Thus, as an example, a log₁₀AUC range between 1.2 μg*hr/mL and 1.6 μg*hr/mL represents a range in which the REO-528 MALT1 inhibitor achieves a decoupling of the efficacy of the MALT1 inhibitor and the reduction of Treg levels.

FIG. 7E depicts clinical score and percent reduction in Tregs following administration of REO-528 MALT1 inhibitor as a function of Cmax. As shown in FIG. 7E, at about a log₁₀Cmax value of 3.3 ng/ml, the REO-528 MALT1 inhibitor exhibits efficacy in the form of ˜50% reduction in the clinical score. Furthermore, at about a log₁₀Cmax value of 3.9 ng/ml, the REO-528 MALT1 inhibitor exhibits reduction of Tregs in the form of ˜40% reduction in Treg levels. Thus, as an example, a log₁₀Cmax range between 3.3 ng/mL and 3.9 ng/mL represents a range in which the REO-528 MALT1 inhibitor achieves a decoupling of the efficacy of the MALT1 inhibitor and the reduction of Treg levels.

FIG. 7F depicts clinical score and percent reduction in Tregs following administration of REO-528 MALT1 inhibitor as a function of Ctrough (trough concentration). As shown in FIG. 7F, at about a log₁₀Ctrough value of 2.2 ng/ml, the REO-528 MALT1 inhibitor exhibits efficacy in the form of ˜50% reduction in the clinical score. Furthermore, at about a log₁₀Ctrough value of 2.6 ng/ml, the REO-528 MALT1 inhibitor exhibits reduction of Tregs in the form of ˜50% reduction in Treg levels. Thus, as an example, a log₁₀Ctrough range between 2.2 ng/ml and 2.6 ng/mL represents a range in which the REO-528 MALT1 inhibitor achieves a decoupling of the efficacy of the MALT1 inhibitor and the reduction of Treg levels.

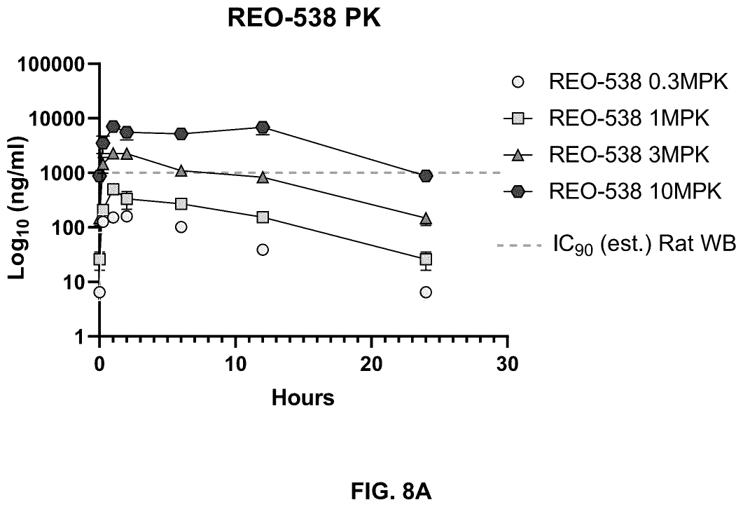

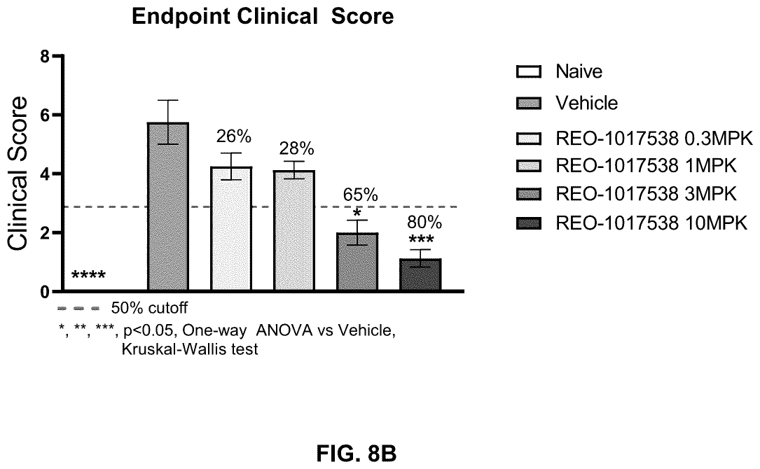

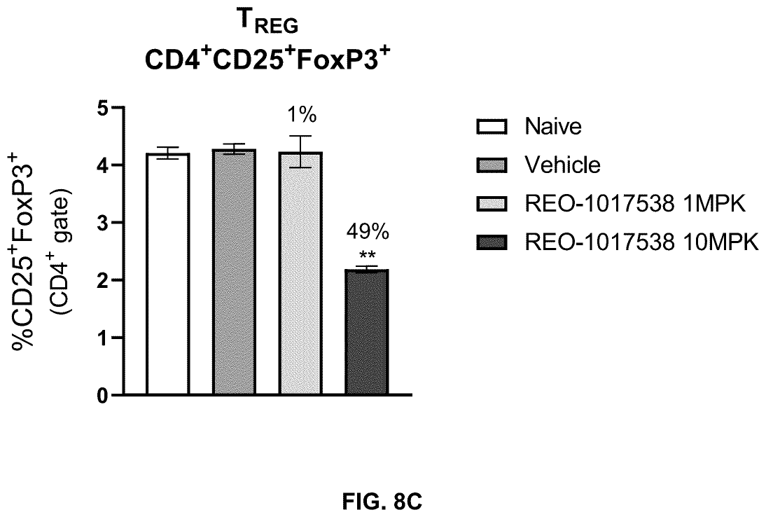

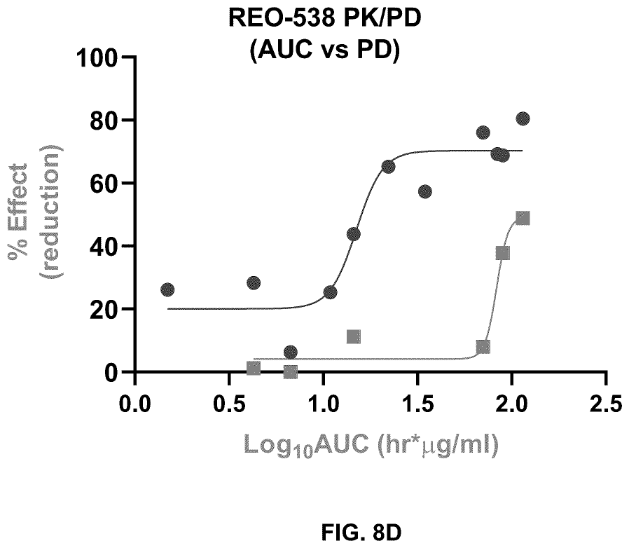

FIG. 8A depicts single dose pharmacokinetics of the REO-538 MALT1 inhibitor in a rat collagen induced arthritis (CIA) model. The REO-538 MALT1 inhibitor was administered in 4 doses (0.3 mg/kg (mpk), 1 mg/kg (mpk), 3 mg/kg (mpk), and 10 mg/kg (mpk)). FIG. 8A shows the blood PK of the REO-538 MALT1 inhibitor up to 24 hours post-administration. Furthermore, FIG. 8A shows the estimated IC90 value (see horizontal dotted line). Here, rats administered 10 mg/kg REO-538 MALT1 inhibitor exhibited blood concentration levels of the MALT1 inhibitor that was above the IC90 value for nearly the full 24 hours. Rats administered the dose of 3 mg/kg REO-538 MALT1 inhibitor exhibited blood concentration levels of the MALT1 inhibitor that was above the IC90 value for more than 6 of the 24 hours.

FIG. 8B depicts endpoint clinical score at various doses of the REO-538 MALT1 inhibitor in a rat collagen induced arthritis (CIA) model. Here, a 0.3 mg/kg dose of REO-538 resulted in a 26% reduction in the clinical score, a 1 mg/kg dose of REO-538 resulted in a 59% reduction in the clinical score, a 3 mg/kg dose of REO-538 resulted in a 65% reduction in the clinical score, and a 10 mg/kg dose of REO-538 resulted in a 80% reduction in the clinical score (reduction in comparison to the vehicle treatment).

FIG. 8C depicts levels of Tregs following administration of REO-538 MALT1 inhibitor in a rat collagen induced arthritis (CIA) model. Here, a 1 mg/kg dose of REO-538 resulted in a 1% reduction in the Treg levels whereas a 10 mg/kg dose of REO-538 resulted in a 49% reduction in the Treg levels (reduced in comparison to vehicle).

FIG. 8D depicts clinical score and percent reduction in Tregs following administration of REO-538 MALT1 inhibitor as a function of area under the curve (AUC). Here, FIG. 8D shows the decoupling of the efficacy of the REO-538 MALT1 inhibitor and the reduction of Treg levels. As shown in FIG. 8D, at about a log₁₀AUC value of 1.2 μg*hr/mL, the REO-538 MALT1 inhibitor exhibits efficacy in the form of ˜50% reduction in the clinical score. Furthermore, at about a log₁₀AUC value of 2.0 μg*hr/mL, the REO-538 MALT1 inhibitor exhibits reduction of Tregs in the form of ˜40% reduction in Treg levels. Thus, as an example, a log₁₀AUC range between 1.2 μg*hr/mL and 2.0 μg*hr/mL represents a range in which the REO-538 MALT1 inhibitor achieves a decoupling of the efficacy of the MALT1 inhibitor and the reduction of Treg levels.

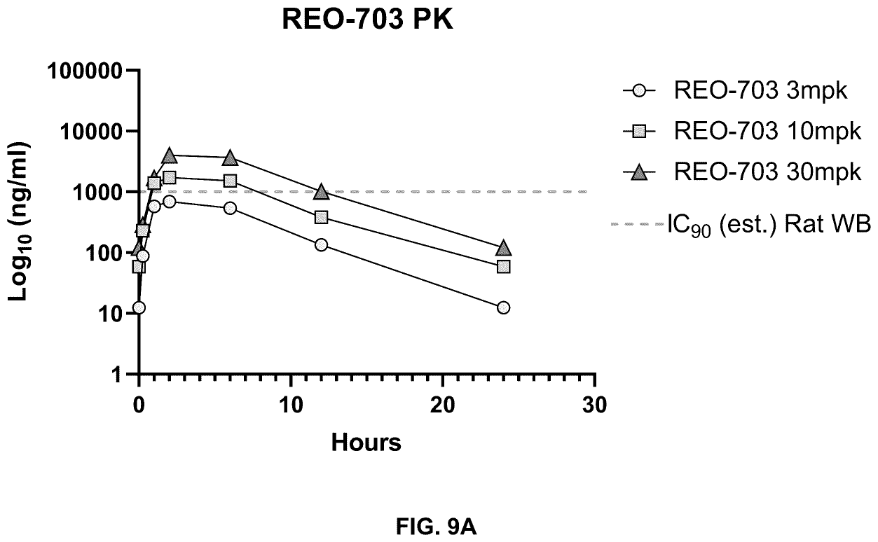

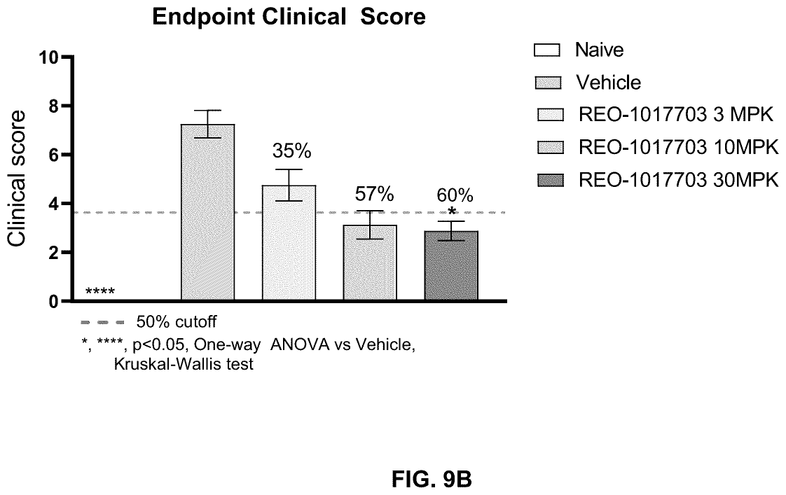

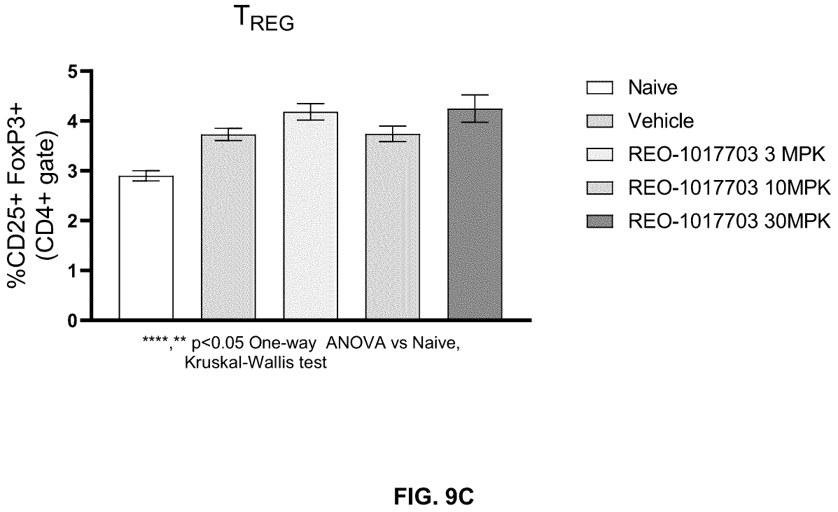

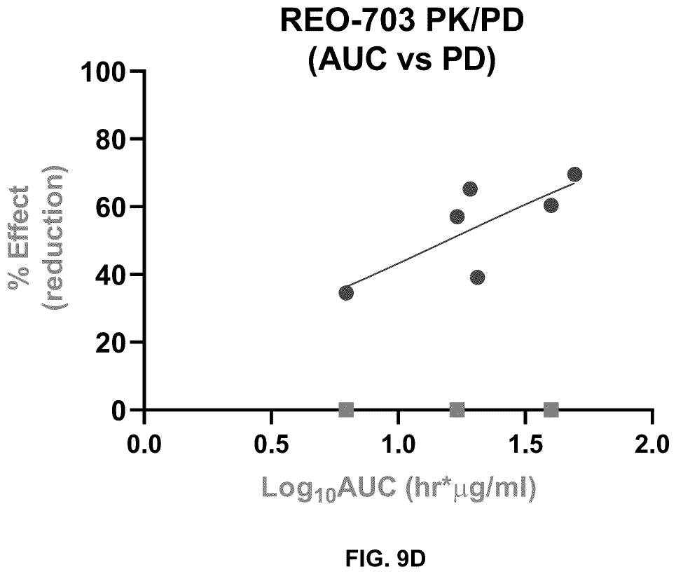

FIG. 9A depicts single dose pharmacokinetics of the REO-703 MALT1 inhibitor in a rat collagen induced arthritis (CIA) model. The REO-703 MALT1 inhibitor was administered in 3 doses (3 mg/kg (mpk), 10 mg/kg (mpk), and 30 mg/kg (mpk)). FIG. 9A shows the blood PK of the REO-703 MALT1 inhibitor up to 24 hours post-administration. Furthermore, FIG. 9A shows the estimated IC90 value (see horizontal dotted line). Here, rats administered 30 mg/kg REO-703 MALT1 inhibitor exhibited blood concentration levels of the MALT1 inhibitor that was above the IC90 value for about 12 hours of the full 24 hours. Rats administered the dose of 10 mg/kg REO-703 MALT1 inhibitor exhibited blood concentration levels of the MALT1 inhibitor that was above the IC90 value for about 6 hours of the 24 hours.

FIG. 9B depicts endpoint clinical score at various doses of the REO-703 MALT1 inhibitor in a rat collagen induced arthritis (CIA) model. Here, a 3 mg/kg dose of REO-703 resulted in a 35% reduction in the clinical score, a 10 mg/kg dose of REO-703 resulted in a 57% reduction in the clinical score, and a 30 mg/kg dose of REO-703 resulted in a 60% reduction in the clinical score (reduction in comparison to the vehicle treatment).

FIG. 9C depicts levels of Tregs following administration of REO-703 MALT1 inhibitor in a rat collagen induced arthritis (CIA) model. Here, none of the doses of REO-703 resulted in a statistically significant reduction in the Treg levels (in comparison to vehicle).

FIG. 9D depicts clinical score and percent reduction in Tregs following administration of REO-703 MALT1 inhibitor as a function of area under the curve (AUC). Here, FIG. 9D shows the decoupling of the efficacy of the REO-703 MALT1 inhibitor and the reduction of Treg levels. As shown in FIG. 9D, at about a log₁₀AUC value of 1.2 μg*hr/mL, the REO-703 MALT1 inhibitor exhibits efficacy in the form of ˜50% reduction in the clinical score. Additionally, the REO-703 MALT1 inhibitor did not exhibit reduction of Tregs. Thus, as an example, a log₁₀AUC range above 1.2 μg*hr/mL represents a range in which the REO-703 MALT1 inhibitor achieves a decoupling of the efficacy of the MALT1 inhibitor and the reduction of Treg levels.

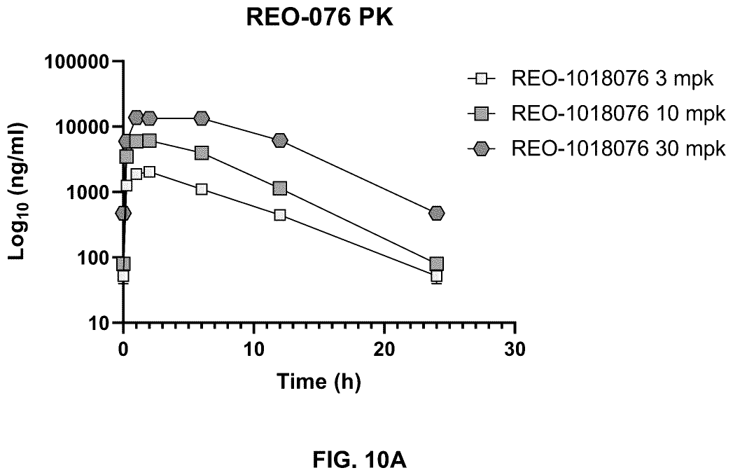

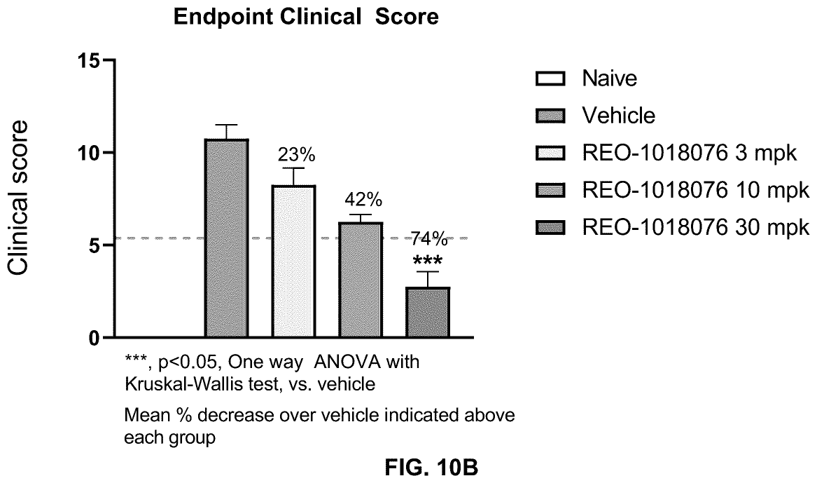

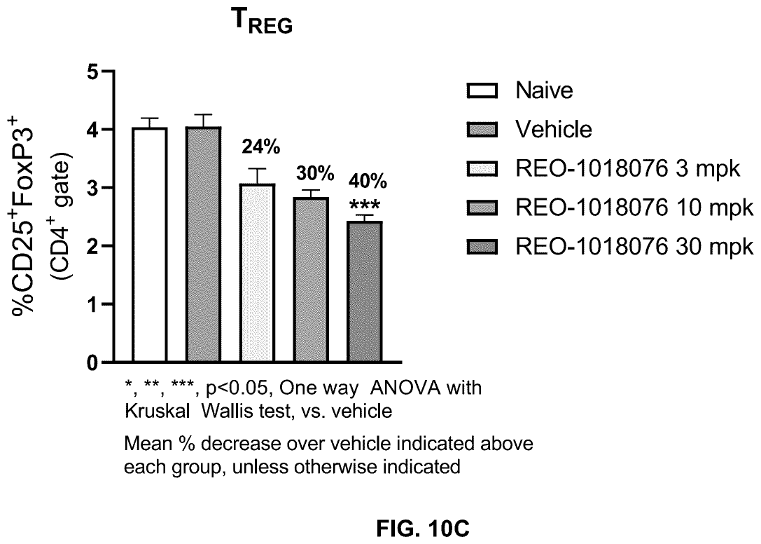

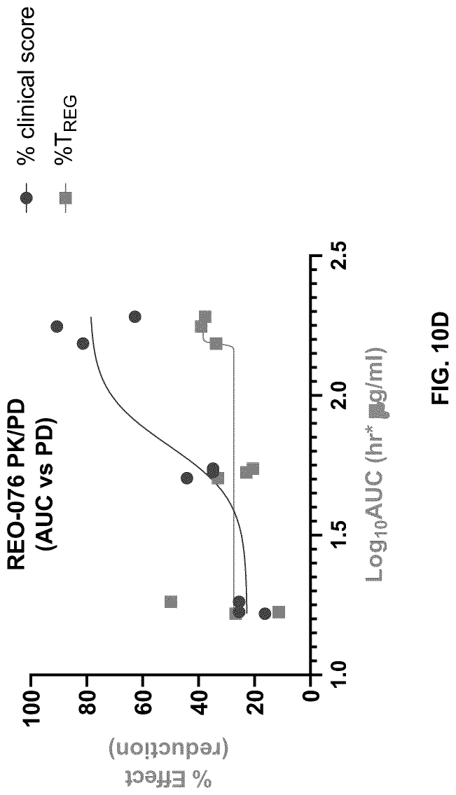

FIG. 10A depicts single dose pharmacokinetics of the REO-076 MALT1 inhibitor in a rat collagen induced arthritis (CIA) model. The REO-076 MALT1 inhibitor was administered in 3 doses (3 mg/kg (mpk), 10 mg/kg (mpk), and 30 mg/kg (mpk)). FIG. 10A shows the blood PK of the REO-076 MALT1 inhibitor up to 24 hours post-administration.

FIG. 10B depicts endpoint clinical score at various doses of the REO-076 MALT1 inhibitor in a rat collagen induced arthritis (CIA) model. The dotted line in FIG. 10B indicates the clinical score corresponding to 50% symptom reduction. Here, a 3 mg/kg dose of REO-076 resulted in a 23% reduction in the clinical score, a 10 mg/kg dose of REO-076 resulted in a 42% reduction in the clinical score, and a 30 mg/kg dose of REO-076 resulted in a 74% reduction in the clinical score (reduction in comparison to the vehicle treatment).

FIG. 10C depicts levels of Tregs following administration of REO-076 MALT1 inhibitor in a rat collagen induced arthritis (CIA) model. Here, a 3 mg/kg dose of REO-076 resulted in a 24% reduction in the Treg levels, a 10 mg/kg dose of REO-076 resulted in a 30% reduction in the Treg levels, and a 30 mg/kg dose of REO-076 resulted in a 40% reduction in the Treg levels (reduced in comparison to vehicle).

FIG. 10D depicts clinical score and percent reduction in Tregs following administration of REO-076 MALT1 inhibitor as a function of area under the curve (AUC). As shown in FIG. 10D, at about a log₁₀AUC value of 1.85 μg*hr/mL, the REO-076 MALT1 inhibitor exhibits efficacy in the form of ˜50% reduction in the clinical score. Furthermore, at about a log₁₀AUC value of 2.25 μg*hr/mL, the REO-076 MALT1 inhibitor exhibits reduction of Tregs in the form of ˜40% reduction in Treg levels. Thus, as an example, a log₁₀AUC range between 1.85 μg*hr/mL and 2.25 μg*hr/mL represents a range in which the REO-076 MALT1 inhibitor achieves a decoupling of the efficacy of the MALT1 inhibitor and the reduction of Treg levels.

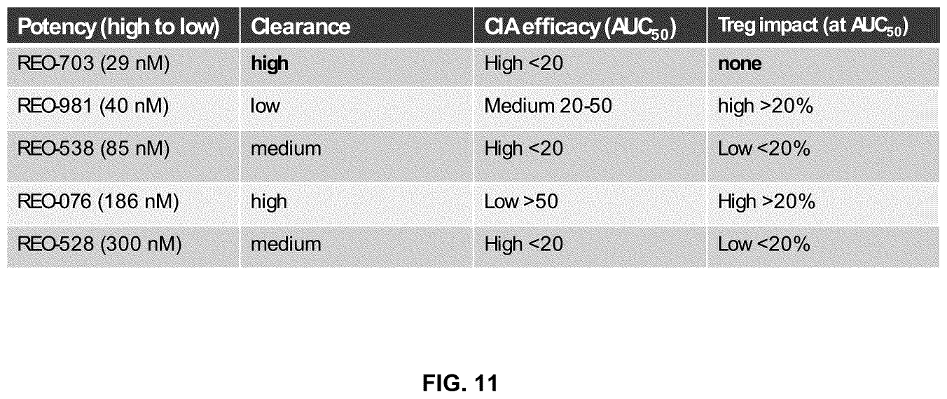

FIG. 11 shows qualitative efficacy and Treg impact results of each of the various MALT1 inhibitors. Of note, REO-538 and REO-528 both exhibit high efficacy and low Treg impact, meaning that they may have larger ranges of log₁₀AUC values in which their efficacy and Treg reduction effects are decoupled.

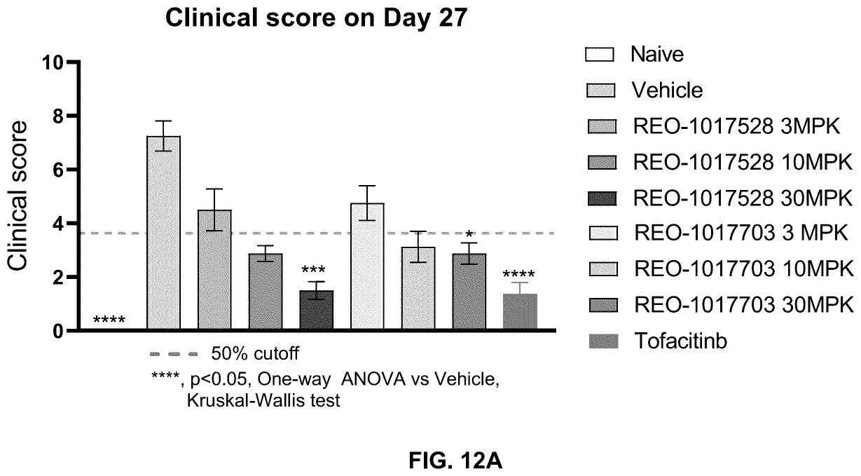

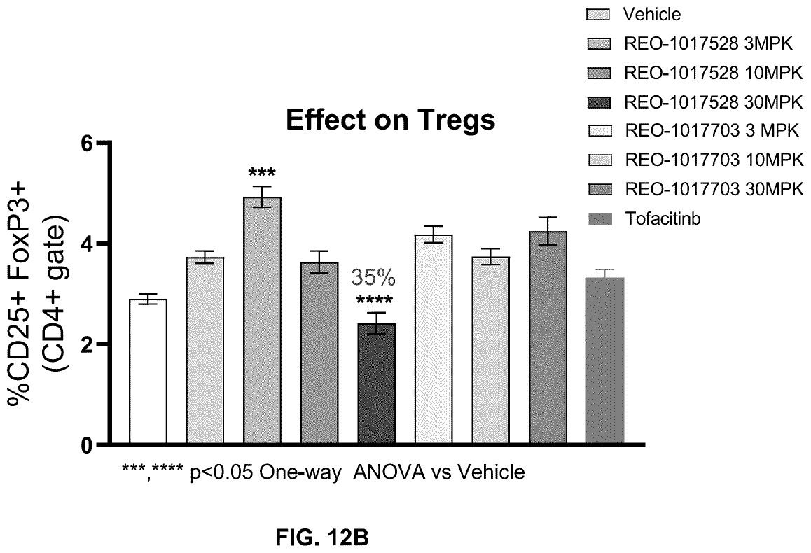

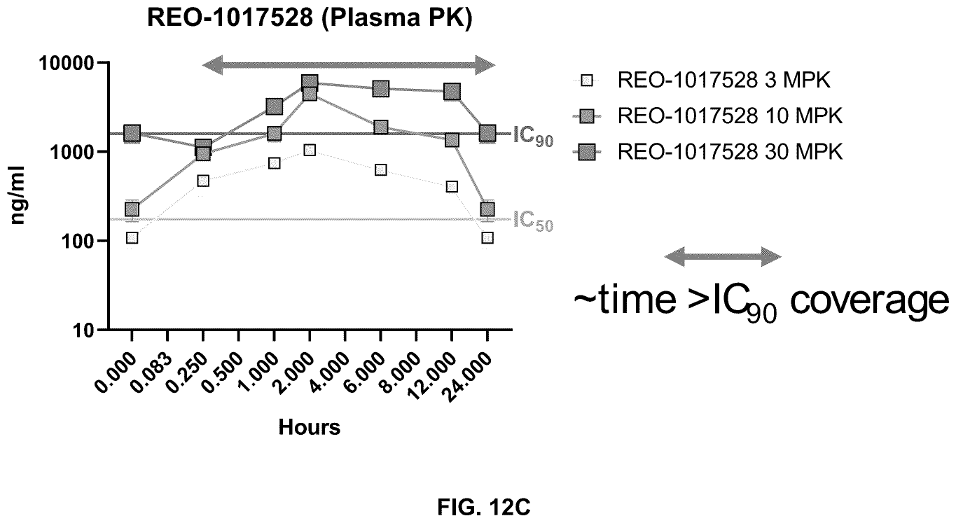

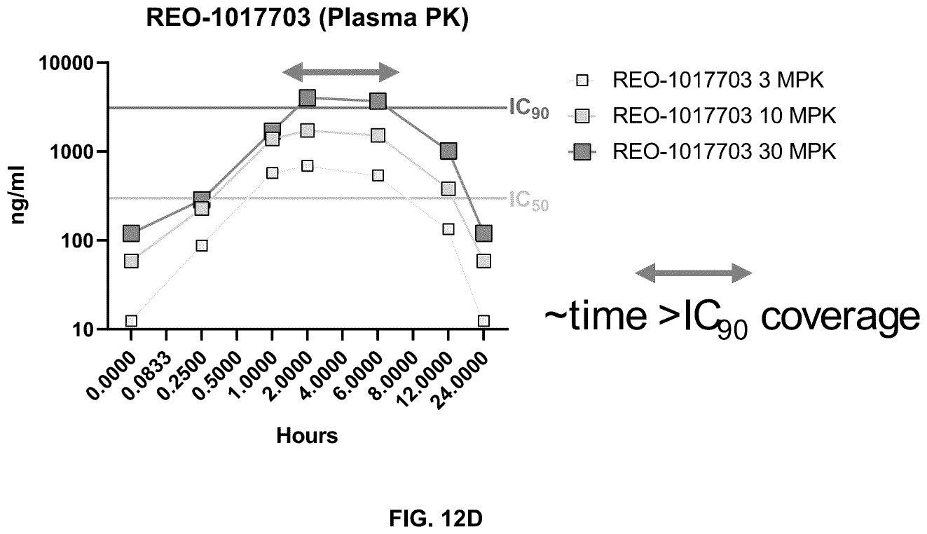

FIG. 12A shows dose-dependent efficacy of REO-528 and REO-703 MALT1 inhibitors in a rat CIA model. Here, both REO-528 and -703 showed efficacy in a rat CIA model in a dose-dependent manner.

FIG. 12B shows levels of Tregs following administration of REO-528 and REO-703 MALT1 inhibitors in a rat CIA model. Notably, a dose-dependent reduction of Tregs was observed for REO-528 but not for REO-703.

FIG. 12C shows plasma pharmacokinetics of REO-528. FIG. 12D shows plasma pharmacokinetics of REO-703. Plasma PK suggests shorter time-on-target (e.g., blood concentration of MALT1 inhibitor above IC90 value) for REO-703 as compared to REO-538. Specifically, at a dose of 30 mg/kg, REO-528 achieved a concentration above the IC90 value for almost all 24 hours post-administration. Comparatively, at a dose of 30 mg/kg, REO-703 achieved a concentration above the IC90 value for about 4 hours within the 24 hours post-administration.

### Example 6: PK and PD of REO-538 MALT1 Inhibitor Administered According to Various Dosing Regimen

In this example, REO-538 MALT1 inhibitor was administered to rats in a collagen induced arthritis (CIA) rat model and analyzed according to the methods described in Example 1.

At time=0, rats were dosed with 1) naïve, 2) vehicle, or 3) MALT1 inhibitor via oral gavage. Rats that were dosed with MALT1 inhibitor were dosed according one of three dosing regimen: 1) daily 3 mg/kg for 14 days (q.d.×14) (referred to in this example as the 3 mg/kg group), 2) daily 10 mg/kg for 14 days (q.d.×14) (referred to in this example as the 10 mg/kg group), 3) daily 10 mg/kg for 2 days (q.d.×2) followed by daily 3 mg/kg for 12 days (q.d.×12) (referred to in this example as the 10+3 mg/kg group). To analyze cytokine levels, a-CD3/CD28 stimulation of 200 ml heparinized whole blood (1:1 diluted) for 24 hours followed by separation of plasma. Cytokines in the plasma and synovial fluid were measured by an electrochemiluminescence based detection method (Meso scale detection, MSD) and via ELISA.

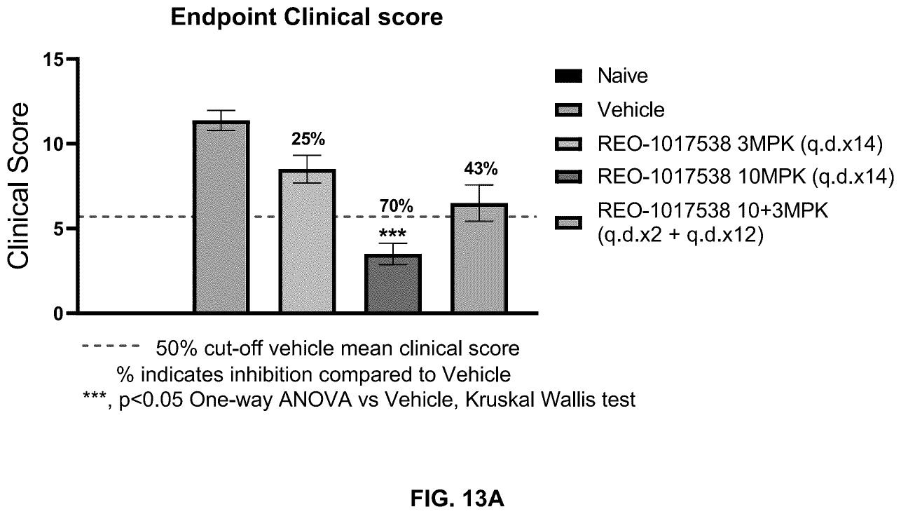

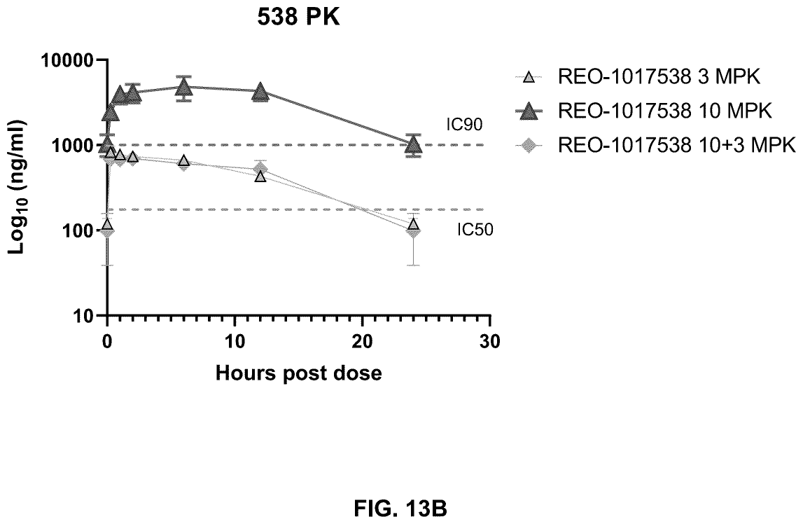

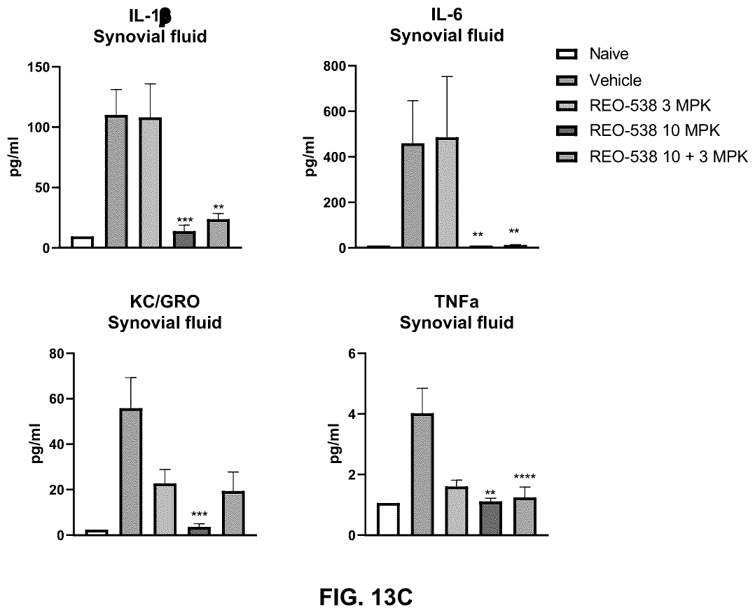

FIG. 13A shows endpoint clinical score across different dosing regimen involving REO-538 MALT inhibitor. Here, the daily 3 mg/kg dose of REO-538 for 14 days resulted in a 25% reduction in the clinical score. The daily 10 mg/kg dose of REO-538 for 14 days resulted in a 70% reduction in the clinical score. The daily 10 mg/kg for 2 days followed by daily 3 mg/kg for 12 days of REO-538 resulted in a 43% reduction in the clinical score (reduction in comparison to the vehicle treatment).

FIG. 13B shows single dose pharmacokinetics of REO-538 across different dosing regimen. FIG. 13B shows the blood PK of the REO-538 MALT1 inhibitor up to 24 hours post-administration (on Day 28). Furthermore, FIG. 13B shows the estimated IC50 value (lower dotted line at log₁₀ value of ˜200 ng/mL) and IC90 value (higher dotted line at log₁₀ value of ˜1000 ng/mL).

FIG. 13C shows levels of IL-1β, IL-6, KC/GRO, and TNFα in synovial fluid following administration of REO-538. Generally, the cytokine levels of IL-1beta and IL-6 in synovial fluid were lower for the 10 mg/kg and the 10+3 mg/kg group in comparison to the 3 mg/kg group, whereas the 3 mg/kg group showed similar levels of IL-1beta and IL-6 in comparison to the vehicle group. Additionally, each of the 3 mg/kg, 10 mg/kg, and 10+3 mg/kg groups exhibited lower levels of KC/GRO and TNFα in comparison to the vehicle group.

### Example 7: Additional Example of Decoupling Efficacy and Treg Reduction Effects of MALT1 Inhibitors

In this example, the REO-528 MALT1 inhibitor was administered to rats in a collagen induced arthritis (CIA) rat model.

Adult female Lewis Rats (180-200 g body weight) were immunized subcutaneously with Type II bovine collagen/Incomplete Freund's Adjuvant emulsion on Day 0 and Day 7. On Day 14 rats were randomized based on clinical disease scores in different treatment groups. Rats received either once daily (q.d) dose of compounds via oral gavage or Vehicle (0.5% Carboxymethylcellulose-Na+0.5% Tween 80 in water, suspension) for 2 weeks. Naïve rats used as controls. Body weight measurements were performed thrice weekly to assess compound tolerability. Clinical score and joint swelling (hind limb volume) were measured on Day 0, before the first dosing and then 3 times a week until the end of the study. Clinical scores measured on a scale of 0-4 per limb are described in the table below.

| Score | Clinical Signs |
|---|---|
| 0 | No evidence of erythema or swelling |
| 1 | Erythema and mild swelling confined to the mid-foot (tarsal) |
| | or ankle joint, or one digit |
| 2 | Erythema and mild swelling extending from ankle to the mid- |
| | foot, or at last two digits |
| 3 | Erythema and moderate swelling extending from ankle to the |
| | metatarsal joints |
| 4 | Erythema and severe swelling encompassing the ankle, foot |
| | and digits |

Following 14 days of dosing, subset of rats were bled at different time intervals (15 min, 1 h, 2 h, 6 h, 12 h and 24 h following last dose) to assess compound exposure levels in the plasma. At study termination on day 28, whole blood CBC was measured. Cytokine levels and anti-collagen antibody levels in plasma were measured by ELISA. Splenocytes were collected and immunophenotyping assessed by flow cytometry.

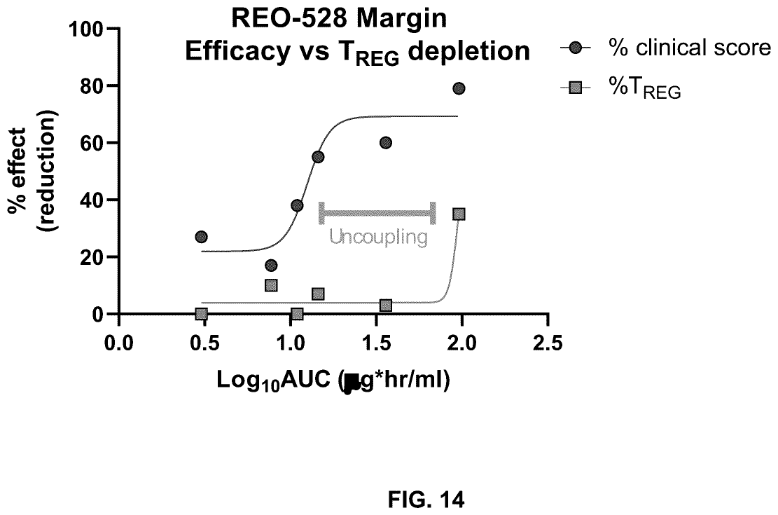

FIG. 14 depicts clinical score and percent reduction in Tregs following administration of REO-528 MALT1 inhibitor as a function of area under the curve (AUC). Here, FIG. 14 shows the decoupling of the efficacy of the REO-528 MALT1 inhibitor and the reduction of Treg levels. As shown in FIG. 14, at about a log₁₀AUC value of 1.2 μg*hr/mL, the REO-528 MALT1 inhibitor exhibits efficacy in the form of ˜50% reduction in the clinical score. Furthermore, at about a log₁₀AUC value of 2.0 μg*hr/mL, the REO-528 MALT1 inhibitor exhibits reduction of Tregs in the form of ˜40% reduction in Treg levels. Thus, as an example, a log₁₀AUC range between 1.2 μg*hr/mL and 2.0 μg*hr/mL represents a range in which the REO-528 MALT1 inhibitor achieves a decoupling of the efficacy of the MALT1 inhibitor and the reduction of Treg levels.

### Example 8: Identifying Candidate Subjects for MALT1 Inhibitor Administration

Test samples are obtained from subjects (e.g., human subjects) at Day 0 to determine whether the subjects are candidate subjects or non-candidate subjects (as described in FIG. 1). Blood samples are obtained, and an immunoassay is performed to determine levels of IL-2 in the blood sample. Quantitative expression values of IL-2 from the test samples are compared to a reference value. Here, the reference value is an average expression of IL-2 in blood samples obtained from healthy patients.

Subjects whose test samples have a quantitative expression value of IL-2 that is above the reference value are categorized as candidate subjects. Subjects whose test samples have a quantitative expression value of IL-2 that is below the reference value are categorized as non-candidate subjects. Candidate subjects are administered MALT1 inhibitor (e.g., a REO-528 MALT1 inhibitor). Following administration of the MALT1 inhibitor to candidate subjects, the decoupled efficacy of the MALT1 inhibitor and the reduction of Tregs is observed. Specifically, a broad range is observed (e.g., log₁₀AUC range representing a range in which the REO-528 MALT1 inhibitor achieves a decoupling of the efficacy of the MALT1 inhibitor and the reduction of Treg levels). Here, candidate subjects (e.g., those with a quantitative expression value of IL-2 that is above the reference value) exhibit more resistance to Treg reduction as a result of the presence of higher IL-2 levels.

### Example 9: Additional Dosing Regimen of MALT1 Inhibitor (REO-528)

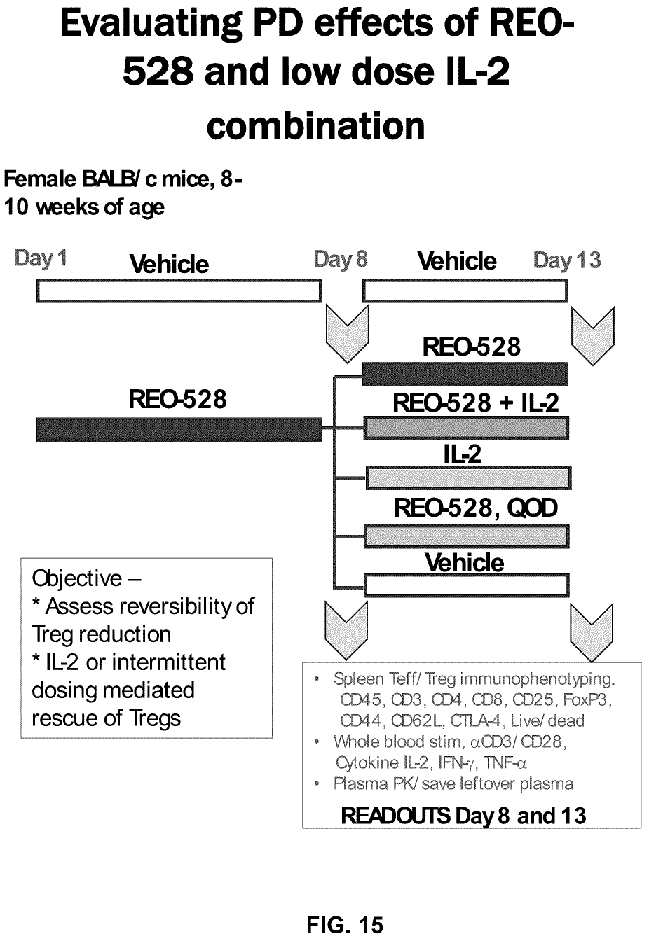

FIG. 15 shows administration strategy for REO-528 MALT1 inhibitor, including combination REO-528+IL-2 therapy. The objective of this administration strategy is to assess the reversibility of Treg reduction following administration of a MALT1 inhibitor and to determine whether IL-2 or intermittent MALT1 inhibitor dosing mediates rescue of Tregs.

At day 1, initiate dosing with vehicle (n=10) or REO-528 compound (n=25) daily via oral gavage at 30 mg/kg (mpk). After 7 days of dosing peel off 5 mice per group (vehicle and compound) within 24 h of last dosing (Day 8) and analyze the following:
Spleen immunophenotyping including Teff and Tregs
a-CD3/CD28 stimulation of 200 ml heparinized whole blood (1:1 diluted) for 24 hours, separate plasma and analyze cytokines; include unstimulated control
Plasma PK analysis for compound only

Continue dosing per schema, 5 mice per new group for 5 days (Days 8-12). Specifically, 1) 5 mice are treated with REO-528 daily, 2) 5 mice are treated with a combination of REO-528 and recombinant murine IL-2 (Peprotech, cat #212-12) administered daily via intraperitoneal route, 30,000 IU per animal (˜2 mg), 3) 5 mice are treated with recombinant murine IL-2 alone, 4) 5 mice are treated with REO-528 every other day (QOD), and 5) 5 mice are treated with vehicle.

After 5 days of dosing (Day 13), sacrifice animals perform similar readouts as on Day 8. Additionally, body weight and body condition of mice are monitored.

### Example 10: Addition of IL-2 Increases IL-2R Signaling Despite MALT1 Inhibition

To assess whether MALT1 inhibition (e.g., using REO-981 MALT1 inhibitor) directly impacted Treg suppressive function an in vitro Treg suppression assay was employed. Tregs were isolated from human donor PBMCs using Human CD4⁺CD127^{low}CD25⁺ Regulatory T-Cell Isolation Kit (Stemcell) and expanded in culture as previously described (16). Briefly, Tregs were activated with Dynabeads Human T-Activator CD3/CD28 (Thermofisher) at a 1:1 bead to cell ratio in complete RPMI media supplemented with 10% FBS, 10 mM HEPES, 2 mM GlutaMAX, 1 mM Na-Pyruvate, and 1×MEM non-essential amino acids (Thermofisher). After 2 days in culture, the culture volume was doubled, and IL-2 was added at a final concentration of 300 IU (Peprotech). At days 5 and 7, cells were expanded in the presence of 300 IU IL-2. On day 9, cells were restimulated with Dynabeads at a 1:1 bead to cell ratio. On day 13, Tregs were harvested and beads were magnetically removed for downstream Treg suppression assays.

For the in vitro Treg suppression assay, naïve CD4⁺ T-cell proliferation was assessed by measuring Cell Trace Violet (CTV) (Thermofisher) dilution in the presence of varying ratios of autologous nTregs. In brief, naïve CD4⁺ T-cells were isolated from human donor PBMCs using the Human Naïve CD4⁺ T-cell Isolation Kit (Stemcell) and labeled with CTV according to manufacturer's protocol. Naïve CD4T⁺-cells were activated with Dynabeads Human T-Activator CD3/CD28 beads at a 1:8 bead to cell ratio for 3 days in the presence of varying ratios of MALT1i-treated nTregs in complete RPMI. Cells were then processed for flow analysis of proliferation. Briefly, cells were first stained with a Fixable Live-Dead dye (BD) and then stained with the following panel of fluorescently labeled antibodies (Biolegend, unless otherwise mentioned): CD4 (clone RPA-T4) and CD25 (clone M-A251), for 20 min at 4° C. Cells were subsequently stained for intracellular FoxP3 (clone 236A/E7, Invitrogen) using the Foxp3 Transcription Factor Staining Buffer Set (eBiosciences) according to the manufacturer's instructions. Cells were analyzed on a BD LSRFortessa flow cytometer. Percent suppression was calculated using the following formula: % suppression=((% naïve T-cell proliferating)−(% naïve T-cell+Treg proliferating))/(% naïve T-cell proliferating)×100.

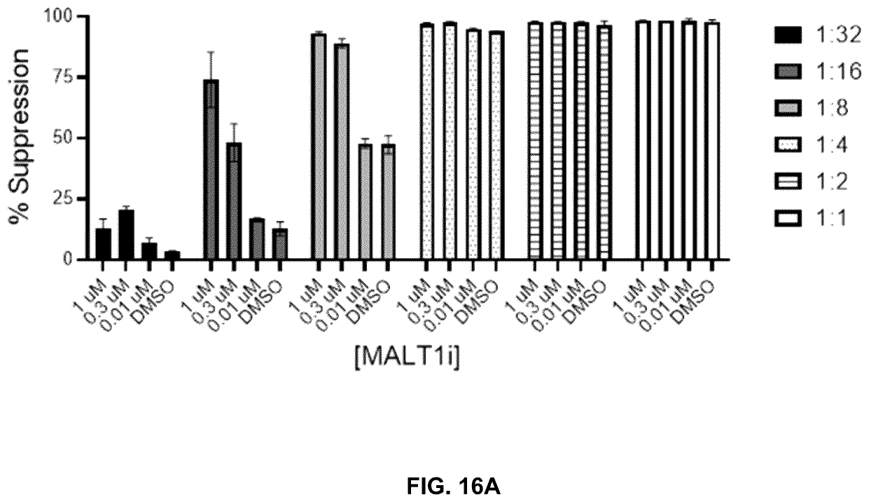

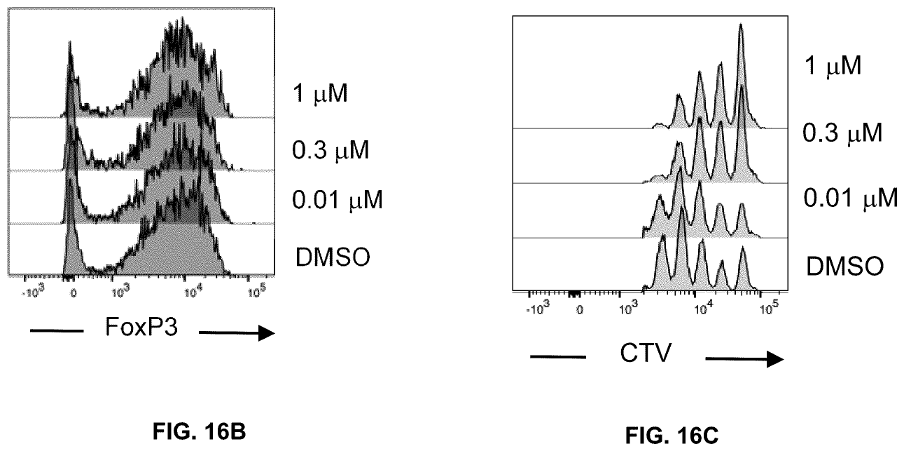

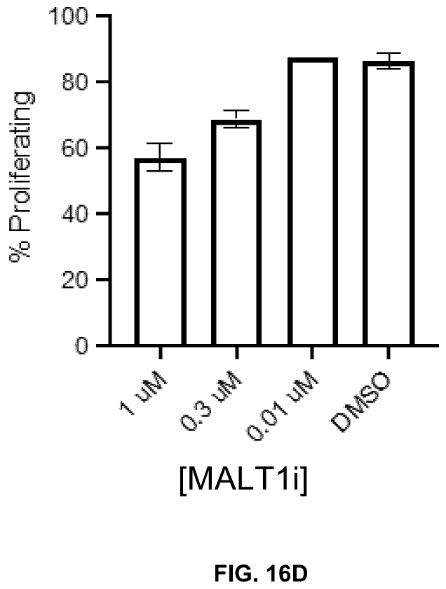

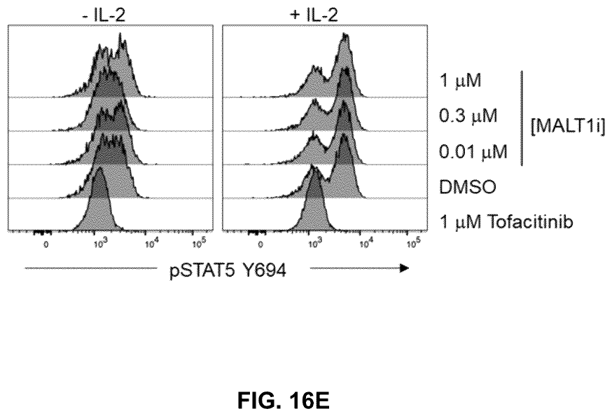

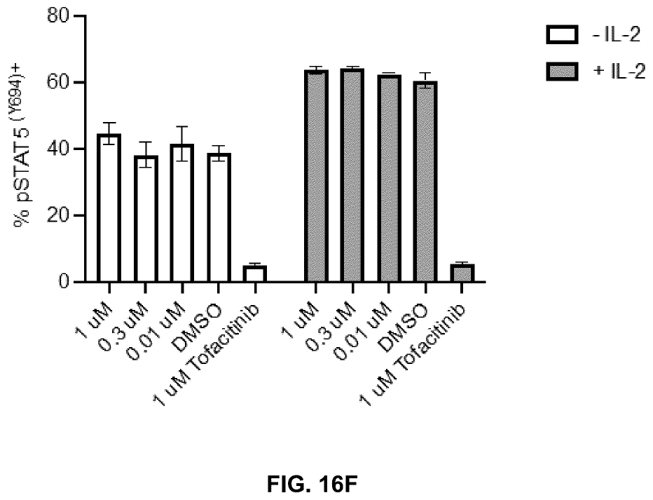

MALT1 inhibition did not reduce the ability of human Tregs to suppress naïve CD4⁺ T-cell proliferation induced by surface CD3/CD28 stimulation using Dynabeads (FIG. 16A). MALT1i also had no effect on FoxP3 expression of Tregs (FIG. 16B). At lower ratios of Tregs to responder CD4⁺ T-cells, higher compound concentrations increased the apparent suppressive capacity of Tregs. This observation likely reflects the effects of the compound on proliferation of the anti-CD3/28 stimulated CD4⁺ T-cells responders. Indeed, labelled responder CD4⁺ T-cells cultured with higher concentrations of MALT1i, and in the absence of co-cultured Tregs, showed decreased proliferative capacity (FIGS. 16C and 16D). Overall, these data suggest that MALT1 inhibition does not directly impact the ability of Tregs to suppress naïve CD4⁺ T-cell proliferation in vitro, nor impact Treg stability via downregulation of FOXP3.

Next, the impact of MALT1i on IL2R signaling was investigated. IL-2R signaling results in phosphorylation of STAT5 (pSTAT5) and is dependent on JAK1/3. Therefore, a flow cytometric readout of pSTAT5 was used to measure IL-2R activity and the JAK1/3-inhibitor tofacitinib served as a positive control for inhibition of pSTAT5.

Purified Tregs were expanded as indicated above. On day 12, cells were harvested and incubated with MALT1i at various concentrations for 18 h in serum-free RPMI at 37° C., 5% CO₂. Tregs were then stimulated with 25 IU IL-2 (Peprotech) for 15 min, followed by fixation with 2% paraformaldehyde. Cells were then permeabilized with 90% methanol and stained with an anti-pSTAT5 antibody (Y694, clone 47, BD) at a 1:100 dilution. Levels of pSTAT5 were analyzed on a BD LSRFortessa flow cytometer.

Interestingly, Tregs appeared to have elevated basal levels of pSTAT5 that were reduced with tofacitinib treatment. IL-2 treatment increased pSTAT5 levels in all conditions except for the tofacitinib-treated group. Importantly, no effects of MALT1i were observed on either basal or IL-2 treated cells, suggesting that MALT1 does not play an important role in this pathway (FIGS. 16E and 16F), and that chronic inhibition of MALT1 spares IL-2R-dependent signaling in Tregs. Furthermore, these results indicate that IL-2 treatment can increase or restore IL-2R dependent signaling in Tregs despite MALT1 inhibition.

### Example 11: Administration of MALT1 Inhibitors Reduces Levels of Tregs

In this example, MALT1 inhibitors, including those of REO-751 and REO-095 were administered to naïve mice to investigate the impact of the MALT1 inhibitor on Treg levels. Mice were dosed for 4 weeks with REO-751 or REO-095 and splenocytes were analyzed for Treg levels. Specifically, REO-751 or REO-095 MALT1 inhibitors were dosed at one of 1 mg/kg (mpk), 10 mg/kg (mpk), or 100 mg/kg (mpk).

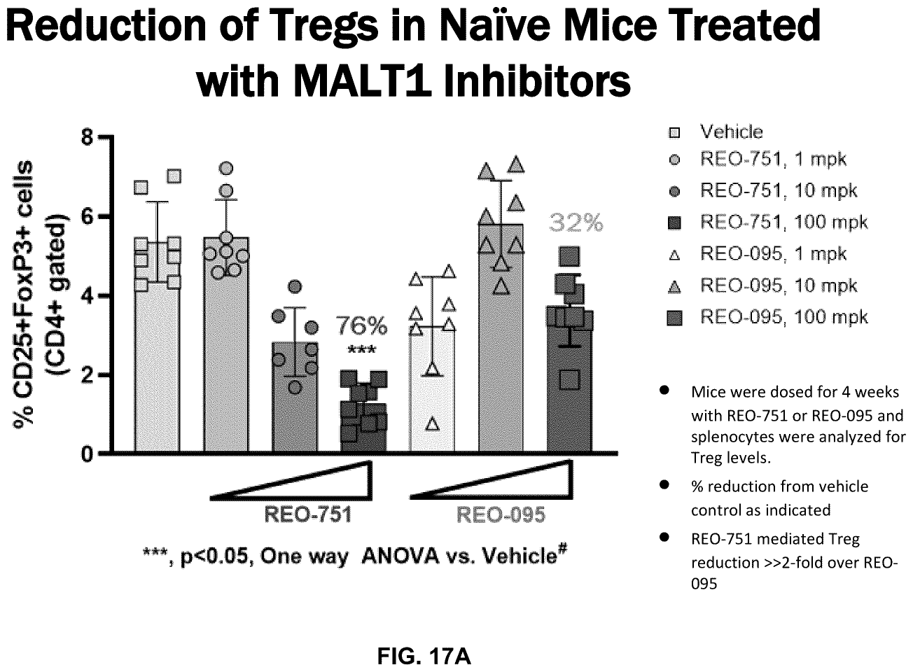

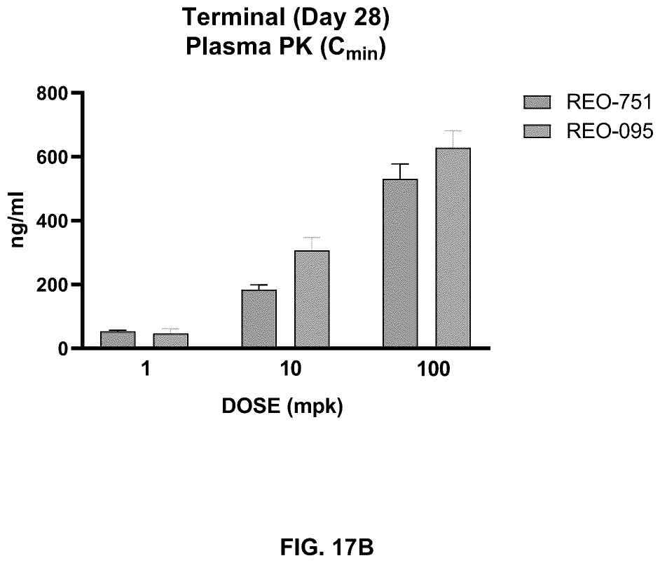

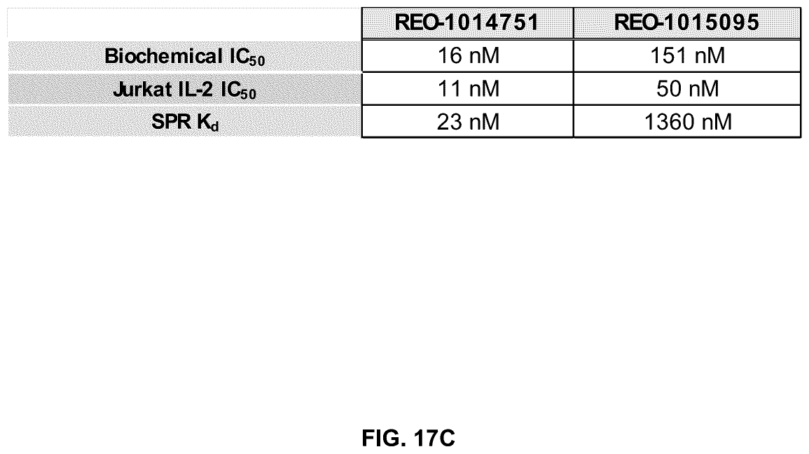

FIG. 17A depicts Treg levels following administration of MALT1 inhibitors in naïve mice. The % reduction of Treg levels are shown in comparison to vehicle control. Generally, REO-751 exhibited higher reduction of Treg levels in comparison to REO-095. In particular, REO-751 mediated Treg reduction >>2-fold over REO-095. At a 100 mg/kg dose, REO-751 reduced Treg levels by 76% whereas at the same 100 mg/kg dose, REO-095 reduced Treg levels by 32%.

Furthermore, the pharmacokinetics (PK) profile of REO-751 and REO-095 were investigated to determine whether the PK profile could explain the difference in Treg reduction. FIG. 17B shows pharmacokinetics profile of MALT1 inhibitors on Day 28. Here, similar exposure levels were observed between both REO-751 and REO-095.

FIG. 17C shows IC50 and Kd values of MALT1 inhibitors. Notably, REO-751 exhibits increased potency (e.g., lower IC50 values and lower Kd value) in comparison to REO-095. Here, the increased potency and affinity can determine the effects on Treg levels. These results suggest that acceptable efficacy can be achieved according to particular potency/affinity properties while ensuring that Treg levels are not overly depleted.

### Example 12: Administration of REO-981 MALT1 Inhibitor for Treatment of Arthritis

Effects of MALT1 inhibitors were analyzed to determine if efficacy could be achieved without impacting the Treg compartment at efficacious concentrations of MALT1i. In this example, MALT1 inhibitors, including REO-981 MALT1 inhibitors, were administered to rats in a collagen induced arthritis (CIA) rat model and analyzed.

Adult female Lewis rats (Charles River) with body weights between 180-200 g body weight were immunized subcutaneously with bovine type II collagen (Chondrex, Woodinville, WA)/Incomplete Freund's Adjuvant (Sigma) emulsion prepared per manufacturer's protocol (Chondrex) on Day 0 and Day 7. MALT1i doses for oral administration were prepared by suspending the compound in 0.5% NA-carboxymethylcellulose/0.5% Tween-80 in water (vehicle). For prophylactic treatment animals were dosed via oral gavage on Day 0 prior to immunization with collagen and continued once daily (q.d.) for four weeks. For therapeutic treatment, animals were randomized per clinical disease scoring on Day 14 and q.d. dosing of compounds via oral gavage was carried out for two weeks. Vehicle treated and naïve animals were used as controls. Clinical score and joint swelling (hind limb volume) were measured on Day 0, before the first dosing and then 3 times a week until the end of the study. Body weight measurements were performed thrice weekly to assess compound tolerability. Criteria (on a scale of 0-4 per limb) were as follows: 0, No evidence of erythema and swelling; 1, Erythema and mild swelling confined to the mid-foot (tarsals) or ankle joint; 2, Erythema and mild swelling extending from the ankle to the mid-foot; 3, Erythema and moderate swelling extending from the ankle to the metatarsal joints; 4, Erythema and severe swelling encompass the ankle, foot, and digits. Following 2-3 weeks of dosing, representative animals were bled at different time intervals over a 0-24 h time-period to assess compound exposure levels in the plasma via LC/MS.

Specifically, REO-981 MALT1 inhibitor was administered to healthy or diseased rats to investigate the impact of the MALT1 inhibitor on Treg levels. Here, diseased rats refer to rats of a collagen induced arthritis (CIA) model. At time=0, rats were dosed with 1) naïve, 2) vehicle, 3) MALT1 inhibitor (1 mg/kg, 3 mg/kg, or 10 mg/kg) or 4) Tofacitinib. Blood was obtained from the rats at various time intervals (e.g., 15 minutes, 1 hour, 2 hours, 6 hours, 12 hours, and 24 hours) post-administration. Blood pharmacokinetics (PK) and pharmacodynamics (PD) of MALT1 inhibitors were calculated from blood samples. Endpoint clinical scores were determined by monitoring the behavior of the rats post-administration. Furthermore, rats were sacrificed at Day 28 post-administration for spleen immunophenotyping including determining levels of Tregs.

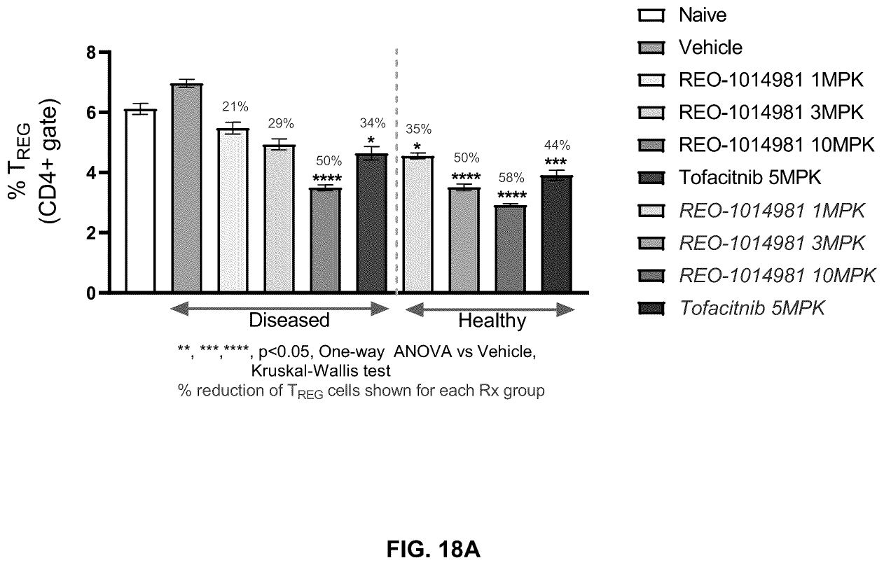

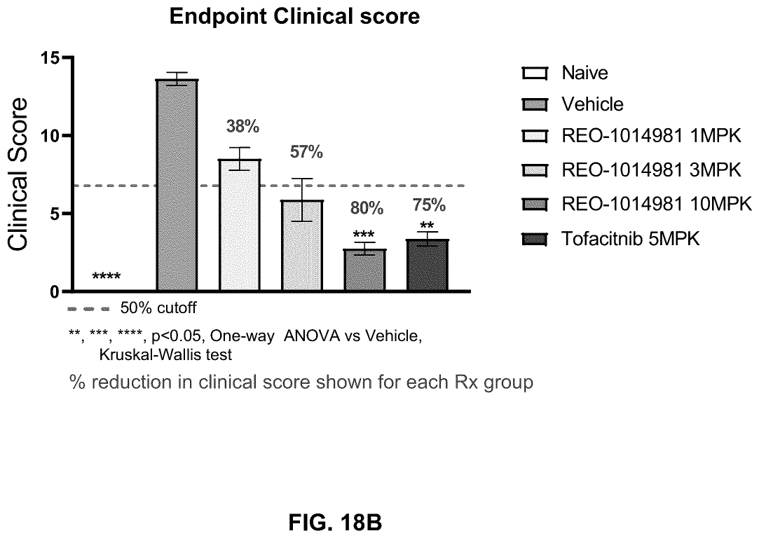

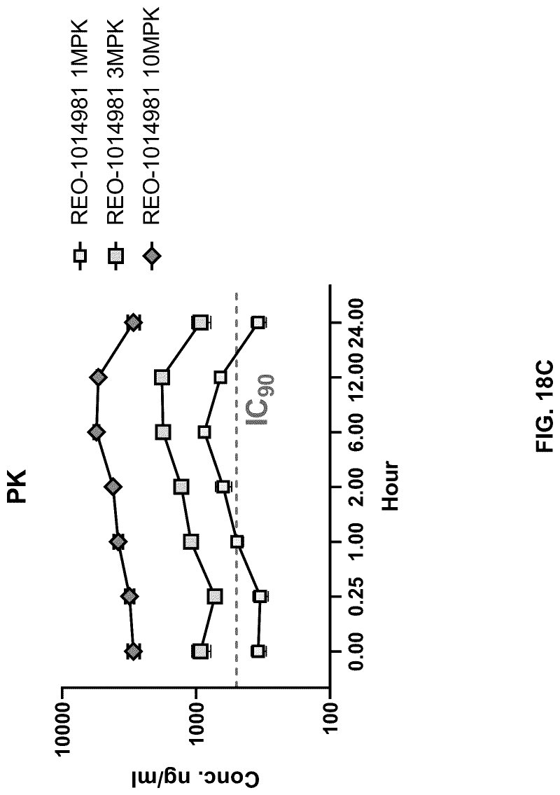

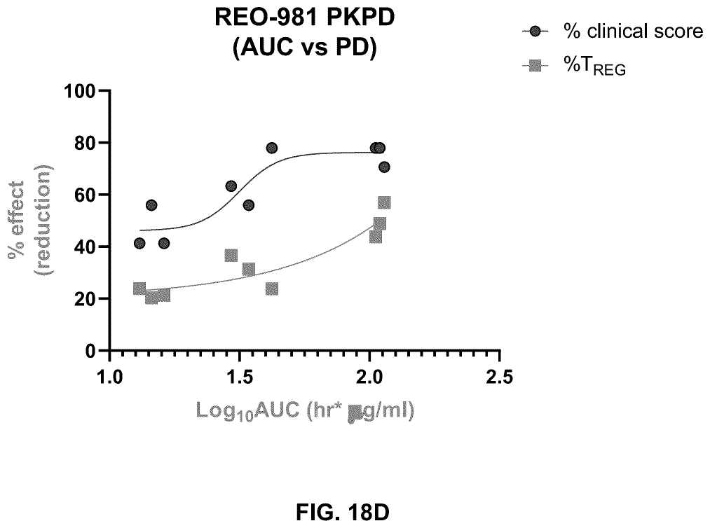

FIG. 18A depicts Treg levels following administration of MALT1 inhibitors in a rat collagen induced arthritis (CIA) model or in healthy animals. Here, a dose-dependent reduction of T-regulatory cells was observed in both diseased and healthy animals treated with REO-981. Of note, diseased rats that received 10 mg/kg REO-981 experienced a 50% reduction in Treg cells, whereas healthy rats that received a lower 1 mg/kg REO-981 dose also experienced a 50% reduction in Treg cells. This suggests that healthy animals are more sensitive to MALT1 inhibition with regards to TREG modulation.

FIG. 18B depicts endpoint clinical scores following administration of MALT1 inhibitors in a rat collagen induced arthritis (CIA) model. Additionally, FIG. 18C depicts the pharmacokinetics (PK) profile following administration of MALT1 inhibitors in a rat collagen induced arthritis (CIA) model. Referring to FIG. 18B, REO-981 achieved a dose-dependent reduction in the clinical score. Administration of a 10 mg/kg REO-981 MALT1 inhibitor achieved an 80% reduction in the endpoint clinical score, similar to the 75% reduction achieved by a 5 mg/kg tofacitinib dose.

Referring to FIG. 18C, both a 3 mg/kg and 10 mg/kg dose of REO-981 achieved a plasma concentration above the IC90 value for the 24 hours post-administration. Additionally, the 1 mg/kg dose of REO-981 achieved a plasma concentration above the IC90 value for ˜12 hours of the 24 hours post-administration.

FIG. 18D depicts clinical score and percent reduction in Tregs following administration of REO-981 MALT1 inhibitor as a function of area under the curve (AUC). Here, FIG. 18D shows the decoupling of the efficacy of the REO-981 MALT1 inhibitor and the depletion of Treg levels. An exposure-response analysis revealed an uncoupling of efficacy in CIA from Treg reduction (FIG. 4D). Specifically, we observed that a drug concentration AUC of 31,500 ng*h/ml achieved a 50% effect on disease score while a drug concentration AUC of 155,000 ng*h/ml reduced Treg numbers by 50% compared to naïve animals (FIG. 18D). Put another way, as shown in FIG. 18D, at about a log₁₀AUC value of 1.5 μg*hr/mL, the REO-981 MALT1 inhibitor exhibits efficacy in the form of ˜50% reduction in the clinical score. Furthermore, at about a log₁₀AUC value of 1.9 μg*hr/mL, the REO-981 MALT1 inhibitor exhibits depletion of Tregs in the form of ˜40% reduction in Treg levels. Thus, as an example, a log₁₀AUC range between 1.5 μg*hr/mL and 1.9 μg*hr/mL represents a range in which the REO-981 MALT1 inhibitor achieves a decoupling of the efficacy of the MALT1 inhibitor and the depletion of Treg levels.

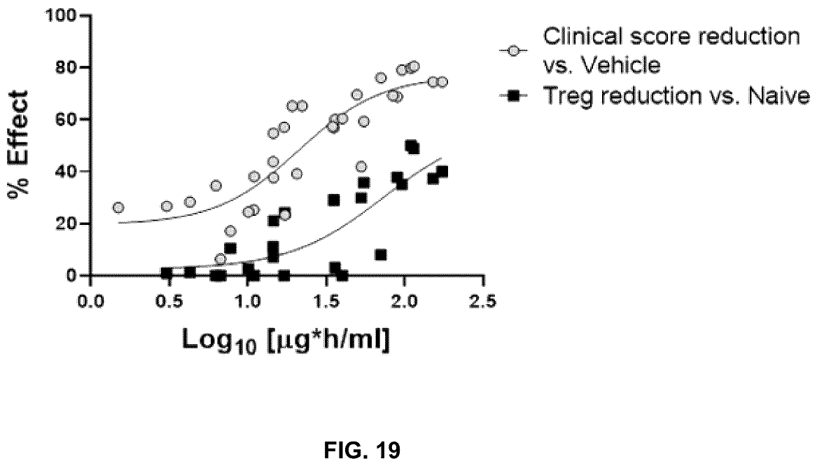

To confirm that this uncoupling of efficacy from Treg reduction was not a function of the specific compound tested, exposure-responses from four distinct MALT1 inhibitors were further analyzed. FIG. 19 shows combined data using 4 structurally distinct MALT1 inhibitors, which depicts uncoupling of efficacy and Treg reduction. Generally, the drug concentrations required to achieve a significant effect on efficacy were 3-5× lower than the drug concentrations required for reduction in Treg numbers (FIG. 19) indicating that uncoupling of efficacy in CIA from reductions in Tregs is a generalizable feature of allosteric inhibition of MALT1.

Altogether, in this example, the impact of REO-981 MATLli on splenic Treg numbers in the CIA model was analyzed to test the impact of MALT1i on Tregs in disease. A statistically significant decrease of CD25⁺FoxP3⁺ Tregs only in rats that receive the highest dose of MALT1i. This was in contrast to disease scores, and to synovial and plasma cytokine and chemokine concentrations, where significant reduction in disease scores was observed at all doses tested. There was further evidence of uncoupling of efficacy from Treg reduction when plasma drug concentrations over time (AUC) were plotted against effects on clinical score and Treg numbers (FIG. 18D). There was a clear separation in the two measured effects: an AUC of 31,500 ng*h/ml resulted in a 50% reduction in disease score while an AUC of 155,000 ng*h/ml was required for a 50% reduction in Treg numbers. These data show that efficacy was achieved at concentrations of MALT1i that were ˜5-fold lower than concentrations that reduced Tregs.

### Example 13: Administration of MALT1 Inhibitors for Treatment of Multiple Sclerosis

In this example, REO-751 MALT1 inhibitor was administered to mice of different disease models (e.g., a prophylactic experimental autoimmune encephalomyelitis (EAE) model, a therapeutic experimental autoimmune encephalomyelitis (EAE)) to investigate the impact of the administration of the MALT1 inhibitor. Induction of EAE in mice is done via injecting with a neuropeptide (MOG35-55) on day 0 and disease in the form of paralysis is detected 7-10 days after. Prophylactic dosing regimen refers to Treatment initiation with MALT1 inhibitor on day 0 prior to induction of disease. Therapeutic dosing regimen refers to treatment initiation with MALT inhibitor on day 12 after disease is detected in the mice. Mice were sampled at endpoint, 24 h post last dose. Clinical scores were determined by monitoring the behavior of the mice post-administration according to the following grading system

| Score | Clinical signs |
|---|---|
| 0 | Normal mouse; no overt signs of disease |
| 1 | Limp tail or hind limb weakness but not both |
| 2 | Limp tail and hind limb weakness |
| 3 | Partial hind limb paralysis |
| 4 | Complete hind limb paralysis |
| 5 | Moribund state; death by EAE: sacrifice for humane reasons |

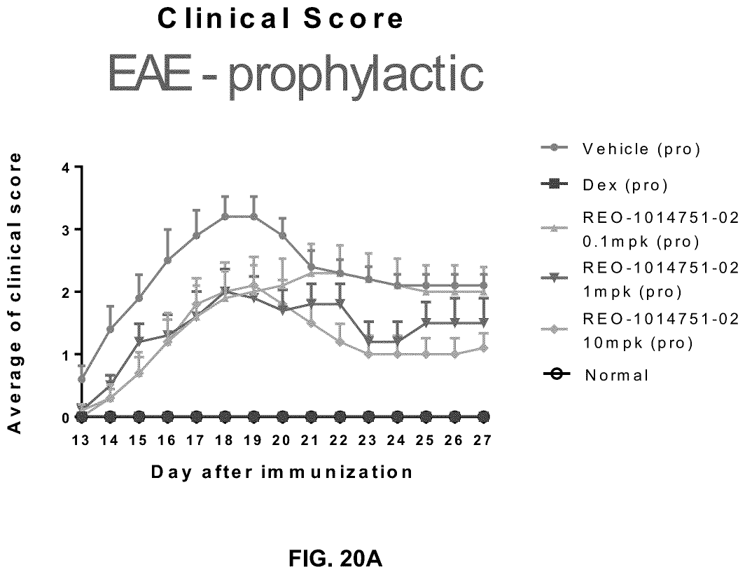

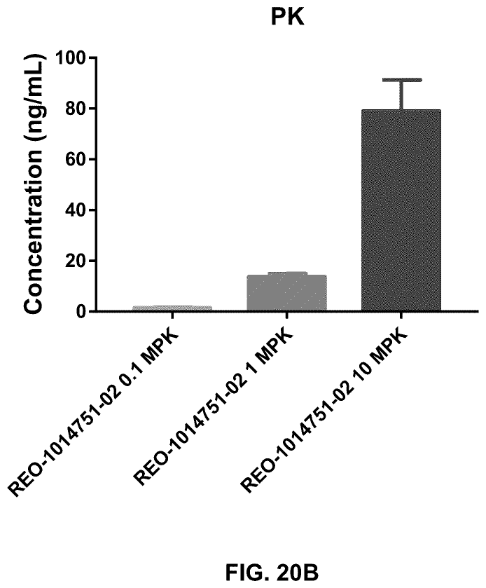

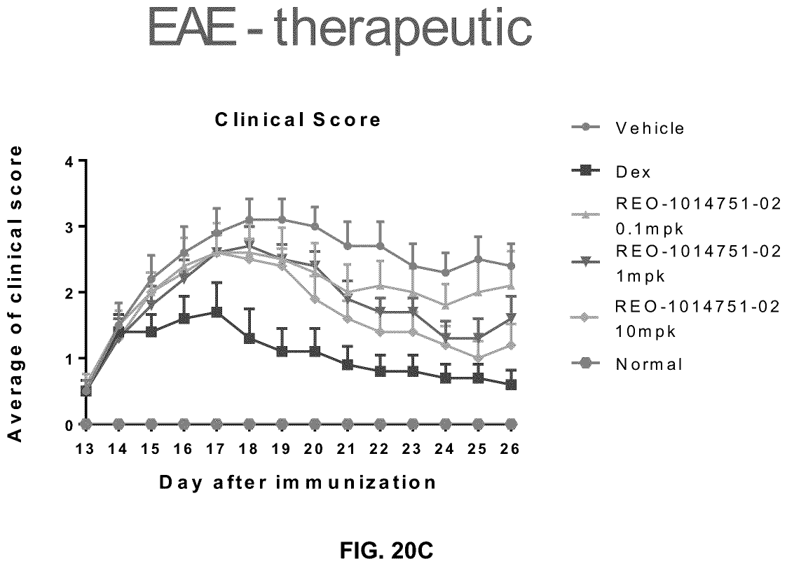

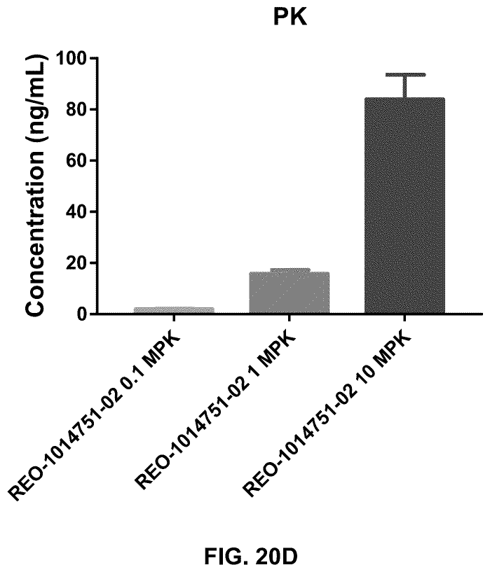

FIG. 20A depicts average clinical score following administration of MALT1 inhibitors in a prophylactic experimental autoimmune encephalomyelitis (EAE) model. FIG. 20B depicts the pharmacokinetics (PK) profile following administration of MALT1 inhibitors in a prophylactic experimental autoimmune encephalomyelitis (EAE) model. FIG. 20C depicts average clinical score following administration of MALT1 inhibitors in a therapeutic experimental autoimmune encephalomyelitis (EAE) model. FIG. 20D depicts the pharmacokinetics (PK) profile following administration of MALT1 inhibitors in a therapeutic experimental autoimmune encephalomyelitis (EAE) model.

Generally, efficacy in the rat EAE model was observed in both prophylactic and therapeutic treatment conditions. Although data is not shown, no effect on Tregs was observed following treatment with MALT1 inhibitors. For a 10 mg/kg REO-951 dose, the concentration of the REO-951 MALT1 inhibitor was ˜50% lower than observed in naïve mice.

### Example 14: Administration of MALT1 Inhibitors for Treatment of Graft Versus Host Disease (GVHD)

In this example, REO-751 MALT1 inhibitor at a dose of 100 mg/kg was administered to mice of a graft versus host disease (GVHD) model to investigate the impact of the administration of the MALT1 inhibitor. The effects of MALT1 inhibitors were evaluated in scGVHD and delayed-type hypersensitivity (DTH) models. For the scGVHD model, bone marrow (1E7 cells) and splenocytes (4E6) from LP/J donors were transferred to C57Bl/6 recipients that had received 8.5. Gy of total irradiation on the previous day. For the DTH model, female Balb/c mice were immunized with keyhole limpet hemocyanin (KLH) subcutaneously on day 0 and challenged with KLH intradermally 7 days later.

At time=0, mice were dosed with 1) naive, 2) vehicle, 3) REO-751 MALT1 inhibitor (100 mg/kg), or 4) ruxolitinib (60 mg/kg). Blood was obtained from the mice at various time intervals (e.g., 15 minutes, 1 hour, 2 hours, 6 hours, 12 hours, and 24 hours) post-administration. Blood pharmacokinetics (PK) and pharmacodynamics (PD) of MALT1 inhibitors were calculated from blood samples. GVHD scores were determined by monitoring the behavior of the mice post-administration according to the following grading system.

| Criteria | Grade 0 | Grade 1 | Grade 2 | Grade 3 | Grade 4 |
|---|---|---|---|---|---|
| Weight loss | no weight loss | >0% < 5% | >5% < 10% | >10% < 15% | <15% |
| Posture | Normal | Mild | Moderate | Severe | — |
| | | hunched | hunched | hunched | |
| | | posture | posture | posture | |
| Activity | Normal | Slowed gait | Slowed gait; | Immobility | — |
| | | | refusal to move | when | |
| | | | when touched | touched | |
| Fur texture | Normal | Ruffled hair; | Hair loss | Hair loss | Complete |
| | | small amount | in a single | in a single | hair loss |
| | | of hair loss | area <1 cm² | area >1 cm² | or <1 cm² |
| | | | | | area involved |
| Skin integrity | Normal | Red/irritated | Skin flaking/ | Scabbing or | Scabbing or |
| | | skin lesion | peeling; | bleeding, | bleeding, |
| | | | single lesion | single lesion | multiple lesions |

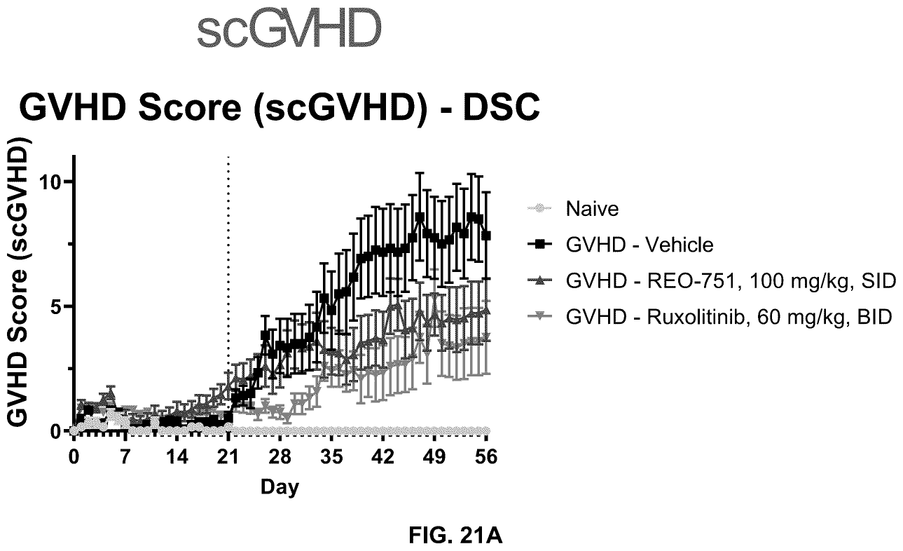

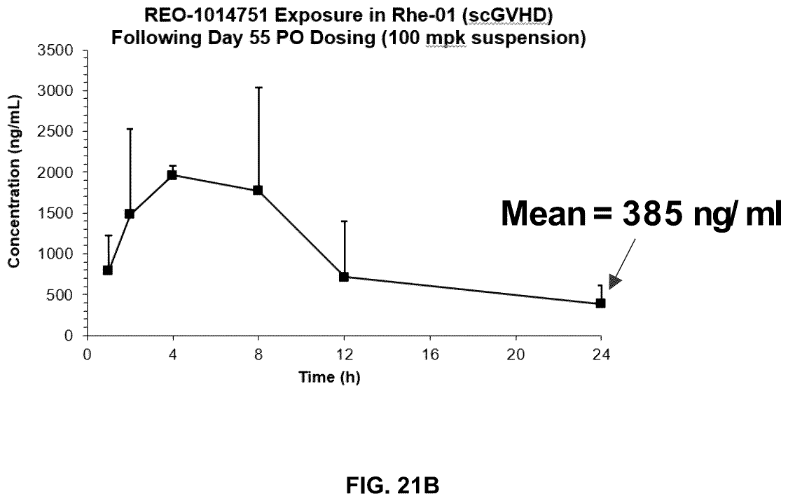

FIG. 21A depicts the average GVHD score following administration of MALT1 inhibitors in a mouse GVHD model. FIG. 21B depicts the pharmacokinetics (PK) profile following administration of MALT1 inhibitors in a mouse GVHD model. Generally, efficacy of the REO-751 MALT1 inhibitor was observed in a GVHD model, as is shown in FIG. 21A. The 100 mg/kg REO-751 dose achieved similar GVHD scores as ruxolitinib (60 mg/kg). No difference in Treg observed with treatment. As shown in FIG. 21B, the concentration of REO-751 at 24 hours post-administration (on Day 55) was 385 ng/ml. This PK concentration at this endpoint is lower than naïve mice.

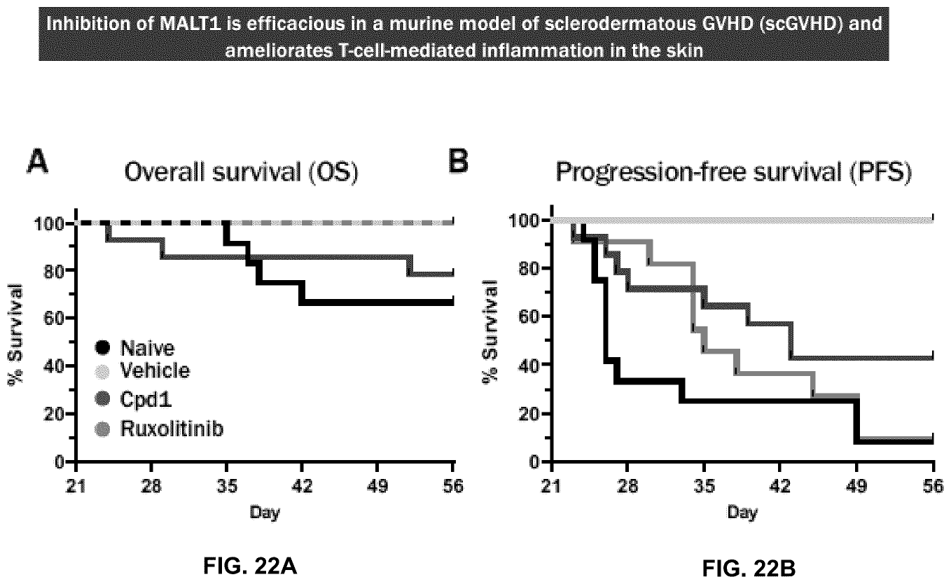

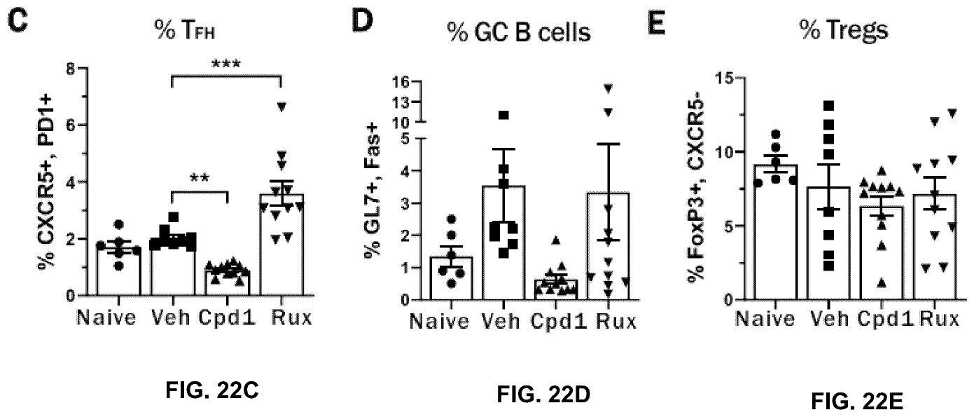

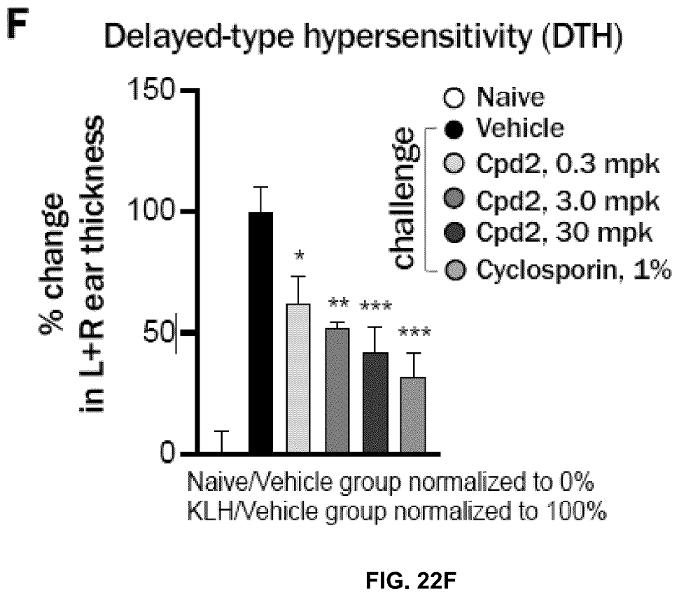

FIGS. 22A-22F show results following administration of a MALT1 inhibitor. Here, “Cpd1” and Cpd2” as shown in FIGS. 22A-22F refer to REO-751 MALT1 inhibitor. FIGS. 22A and 22B show overall survival (OS) and progression-free survival (PFS) Kaplan Meier curves, respectively, in a murine model of sclerodermatous GVHD (scGVHD) following administration of a MALT1 inhibitor. Specifically, FIG. 22A shows the survival for all animals during the scGVHD study in which mice received 1) Vehicle, 2) 100 mg/kg REO-751 orally once daily, or 3) 60 mg/kg ruxolitinib twice daily from days 21-56 (post-cell-transfer). The naïve control group did not receive either irradiation or a cell transfer. Referring to FIG. 22B, it shows the PFS which is defined as an increase in GVHD score of greater than 2 in comparison to Day 21 GVHD score. Here, 40% of mice achieved progression free survival at the end (e.g., Day 56) which represents an improvement over the naïve group and ruxolitinib group.

FIGS. 22C-22E shows levels of T follicular helper cells (T_{FH}) cells, Germinal Center (GC) B cells, and Treg cells in each of the different groups. Here, FIGS. 22C-22E show percentages of each cell population in spleen, as measured by flow cytometry. Notably, the administration of the MALT1 inhibitor reduced percentage of T_{FH} cells and reduced the percentage of GC B cells in comparison to vehicle and ruxolitinib, but did not reduce the percentage of Tregs in comparison to vehicle and ruxolitinib.

Reference is now made to FIG. 22F, which shows percentage change in ear thickness as a measure of delayed-type hypersensitivity following administration of a MALT1 inhibitor. Specifically, FIG. 22F shows ear thickness at 48 hrs post-KLH-challenge, normalized to pre-challenge (per-mouse), and Naive/Vehicle values. FIG. 22F shows that the administration of the MALT1 inhibitor abrogated a DTH response, showing that T-cell-driven responses in the skin, an important target tissue in scGVHD, can be dampened with MALT1 blockade.

### Example 15: Administration of MALT1 Inhibitors for Treatment of Lupus

In this example, REO-528 MALT1 inhibitor was administered to mice of an accelerated Lupus Nephritis model (IFN-alpha accelerated) to investigate the impact of the administration of the MALT1 inhibitor. Mice were divided into 6 groups: 1) Naïve, 2) vehicle (weeks 13-18), 3) REO538 (3 mg/kg) daily administration weeks 13-18, 4) REO-538 (30 mg/kg) daily administration weeks 13-18, 5) REO-538 (30 mg/kg) daily administration weeks 15-18, and 6) BID dexamethasone (Dex) at 1 mg/kg for weeks 15-18. Groups 2 through 6 were also administered AAV encoding mIFNα to accelerate the lupus nephritis disease. Mice were sacrificed post-administration for spleen immunophenotyping including determining levels of Tregs.

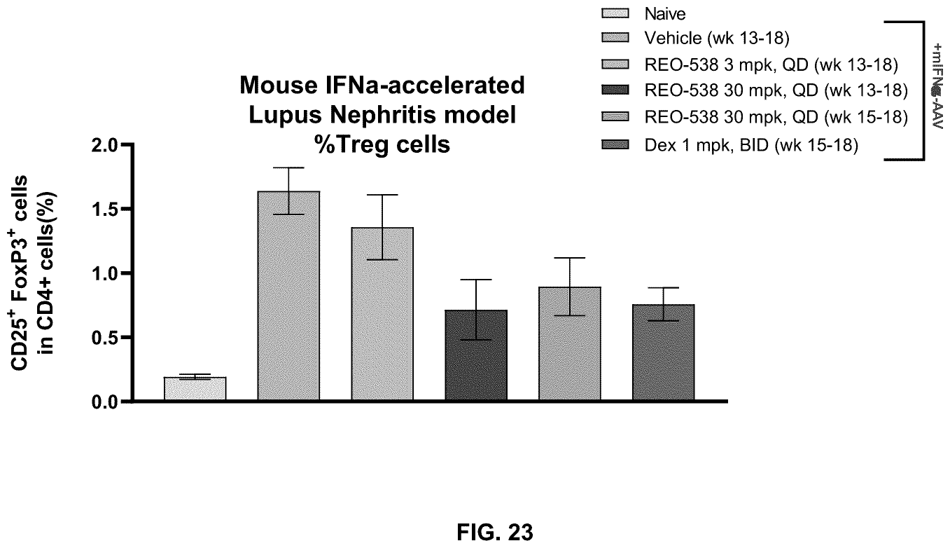

FIG. 23 depicts Treg levels following administration of REO-538 in a mouse accelerated Lupus model. REO-538 reduced Treg levels in a dose dependent manner (e.g., 30 mg/kg REO-538 reduced Treg levels more than 3 mg/kg REO-538). Additionally, % Treg cells (in splenocytes) trend higher in vehicle treated accelerated lupus animals. MALT1 treatment reduces Treg in relation to vehicle but are restored towards naïve levels.

1. A method for treating a chronic disorder, the method comprising administering to the subject a MALT1 inhibitor, wherein following administration to the subject, the MALT1 inhibitor achieves a time over an IC50 blood concentration target from about 4 hours to about 24 hours per 24 hours.

2. The method of claim 1, wherein following administration to the subject, the MALT1 inhibitor achieves a time over the blood concentration target from about 12 hours to about 24 hours per 24 hours.

3. The method of claim 1, wherein following administration to the subject, the MALT1 inhibitor achieves a time over the blood concentration target from about 4 hours to about 20 hours per 24 hours.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A method for treating a chronic disorder, the method comprising administering to the subject a MALT1 inhibitor, wherein following administration to the subject, the MALT1 inhibitor achieves a time over a IC90 blood concentration target from about 6 hours to about 24 hours per 24 hours.

18. (canceled)

19. (canceled)

20. (canceled)

21. (canceled)

22. (canceled)

23. (canceled)

24. A method for treating a chronic disorder, the method comprising administering to the subject a MALT1 inhibitor, wherein following administration to the subject, the MALT1 inhibitor achieves a log₁₀(AUC) from about 0.5 μg*hr/mL to about 2.0 μg*hr/mL.

25. (canceled)

26. (canceled)

27. The method of claim 24, wherein following administration of the MALT1 inhibitor to the subject, a level of Tregs of the subject remains at least 60% of a level prior to administration.

28. The method of claim 24, wherein following administration of the MALT1 inhibitor to the subject, a level of Tregs of the subject remains at least 70% of a level prior to administration.

29. The method of claim 24, wherein following administration of the MALT1 inhibitor to the subject, a level of Tregs of the subject remains at least 80% of a level prior to administration.

30. The method of claim 24, wherein following administration of the MALT1 inhibitor to the subject, a level of Tregs of the subject remains at least 90% of a level prior to administration.

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The method of claim 31, wherein the MALT1 inhibitor is administered intravenously.

46. (canceled)

47. (canceled)

48. (canceled)

49. (canceled)

50. (canceled)

51. (canceled)

52. (canceled)

53. (canceled)

54. The method of claim 24, wherein reduction of a level of Tregs of the subject with the chronic disorder following administration of the MALT1 inhibitor is lower in comparison to reduction of a level of Tregs of a healthy subject who receives the MALT1 inhibitor.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. The method of claim 1, wherein the chronic disorder is graft-versus-host disease (GHVD).

72. (canceled)

73. The method of claim 1, wherein the chronic disorder is psoriatic arthritis.

74. (canceled)

75. (canceled)

76. The method of claim 1, wherein the chronic disorder is inflammatory bowel disease.

77. The method of claim 1, wherein the inflammatory bowel disease is Crohn's Disease.

78. The method of claim 1, wherein the inflammatory bowel disease is ulcerative colitis.

79. The method of claim 1, wherein the chronic disorder is psoriasis.

80. The method of claim 1, wherein the chronic disorder is Lupus, systemic lupus erythematosus, or lupus nephritis.

81. (canceled)

82. (canceled)

83. The method of claim 1, wherein the chronic disorder is rheumatoid arthritis.

84. (canceled)

85. The method of claim 80, wherein the chronic disorder is lupus nephritis.
